SEQUENCE LISTING (16 sequences)

SEQ ID NO: 1 - DNA, 2268 bp, Artificial Sequence
  Human FCGR1A mRNA sequence
aatatcttgcatgttacagatttcactgctcccaccagcttggagacaacatgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcatgtccttttctatctggcagtgggaataatgtttttagtgaacactgttctctgggtgacaatacgtaaagaactgaaaagaaagaaaaagtgggatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtagcagcggctcagtgggtggccatcgatctggaccgtcccctgcccacttgctccccgtgagcactgcgtacaaacatccaaaagttcaacaacaccagaactgtgtgtctcatggtatgtaactcttaaagcaaataaatgaactgacttcaactgggatacatttggaaatgtggtcatcaaagatgacttgaaatgaggcctactctaaagaattcttgaaaaacttacaagtcaagcctagcctgataatcctattacatagtttgaaaaatagtattttatttctcagaacaaggtaaaaaggtgagtgggtgcatatgtacagaagattaagacagagaaacagacagaaagagacacacacacagccaggagtgggtagatttcagggagacaagagggaatagtatagacaataaggaaggaaatagtacttacaaatgactcctaagggactgtgagactgagagggctcacgcctctgtgttcaggatacttagttcatggcttttctctttgactttactaaaagagaatgtctccatacgcgttctaggcatacaagggggtaactcatgatgagaaatggatgtgttattcttgccctctcttttgaggctctctcataacccctctatttctagagacaacaaaaatgctgccagtcctaggcccctgccctgtaggaaggcagaatgtaactgttctgtttgtttaacgattaagtccaaatctccaagtgcggcactgcaaagagacgcttcaagtggggagaagcggcgataccatagagtccagatcttgcctccagagatttgctttaccttcctgattttctggttactaattagcttcaggatacgctgctctcatacttgggctgtagtttggagacaaaatattttcctgccactgtgtaacatagctgaggtaaaaactgaactatgtaaatgactctactaaaagtttagggaaaaaaaacaggaggagtatgacacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - DNA, 1762 bp, Artificial Sequence
  Human ZNF296 mRNA sequence
cgccgtcagggccccagcagggcggctcggtcccctcccatgagccccgccctggtaggcggagccacgggcccggccagcacgtgtaagagttcgcggcgggtcgccgcagtcactcacctgagcgcgcacggtccgcgcgtcctccgctcgtgcgtcctccgcccgcccgcctgcctgcctgcccgcccgctcgctcgcccggcccgcgactcatgtcccgccgcaaggccggcagcgcgccccgccgagtagagcccgcgcccgccgccaacccagacgacgagatggaaatgcaggacctcgtcatcgaactcaagcccgagccagacgcgcagccccaacaggccccaaggctggggcccttctccccgaaggaggtgtcctcggcggggcggttcggcggcgaaccccaccactcccctggccccatgcccgccggggccgccctcctcgccctcggcccgcggaacccgtggaccctgtggacgccgttgaccccgaactatcccgaccgccagccctggaccgacaaacacccagatctgttgacctgcggccgctgcctgcagaccttcccgttggaggccatcactgccttcatggaccacaagaagctgggctgtcagctcttcagaggccccagccgcggccagggctcagaacgagaggagctgaaggccttgagctgcctgcgctgtggcaaacagttcacagtggcctggaagctgctgcgtcacgcccagtgggaccacggactgtccatctaccagacagaatcagaggccccggaggccccgctcctgggcctggccgaggtggctgcagccgtgtcggcagtggtggggccagcagctgaggccaagagcccccgtgcaagtggcagcggcctcacccggcggagccccacctgtcctgtgtgcaagaagaccctcagctccttcagcaacctcaaagtgcacatgcgctcacacacaggcgagcggccctatgcttgcgaccagtgtccctacgcctgcgcccagagcagcaagctcaaccgccacaagaagacccaccggcaggtgccgccccagagccccctcatggccgacaccagccaggagcaggcctctgcagcccctccggagccggctgtccatgctgctgcccccaccagcacccttccatgcagcggtggtgagggggctggagccgccgccacagcaggtgtccaggaacccggggctcctggcagtggggctcaagccggccctggtggagacacttggggagccatcaccacggaacaaagaactgaccctgcaaacagccagaaggcatcacccaaaaagatgcccaagtcagggggcaagagccgcgggcccgggggcagctgtgagttctgcgggaagcattttaccaacagcagcaacctgacggtgcaccggcgctcacacaccggggagcgcccctacacctgtgagttctgcaactacgcctgcgcccagagcagtaagctcaaccgccaccgccgcatgcacggcatgacgcctggcagcacccgcttcgagtgcccccactgccatgtgcccttcggcctgcgagccaccctggacaaacacctgcggcagaagcaccctgaggcggccggcgaggcctgagcccaggaaagcccccctcactgtccctggtaccgctgccaacacccattgacctcctcgtttttgcccgccttctccaagtaaattttcccttttatttaaaaaaaaaaaaaaaaaaa

SEQ ID NO: 3 - DNA, 1044 bp, Artificial Sequence
  Human C1QB mRNA sequence
gcccttcccgcctctggggaagggaacttccgcttcggaccgagggcagtaggctctcggctcctggtcccactgctgctcagcccagtggcctcacaggacaccagcttcccaggaggcgtctgacacagtatgatgatgaagatcccatggggcagcatcccagtactgatgttgctcctgctcctgggcctaatcgatatctcccaggcccagctcagctgcaccgggcccccagccatccctggcatcccgggtatccctgggacacctggccccgatggccaacctgggaccccagggataaaaggagagaaagggcttccagggctggctggagaccatggtgagttcggagagaagggagacccagggattcctgggaatccaggaaaagtcggccccaagggccccatgggccctaaaggtggcccaggggcccctggagccccaggccccaaaggtgaatcgggagactacaaggccacccagaaaatcgccttctctgccacaagaaccatcaacgtccccctgcgccgggaccagaccatccgcttcgaccacgtgatcaccaacatgaacaacaattatgagccccgcagtggcaagttcacctgcaaggtgcccggtctctactacttcacctaccacgccagctctcgagggaacctgtgcgtgaacctcatgcgtggccgggagcgtgcacagaaggtggtcaccttctgtgactatgcctacaacaccttccaggtcaccaccggtggcatggtcctcaagctggagcagggggagaacgtcttcctgcaggccaccgacaagaactcactactgggcatggagggtgccaacagcatcttttccgggttcctgctctttccagatatggaggcctgacctgtgggctgcttcacatccaccccggctccccctgccagcaacgctcactctacccccaacaccaccccttgcccaaccaatgcacacagtagggcttggtgaatgctgctgagtgaatgagtaaataaactcttcaaggccaaggga

SEQ ID NO: 4 - DNA, 4867 bp, Artificial Sequence
  Human GBP6 transcript variant 1 mRNA sequence
acccaacacccatcctcaaactcacatcggatgattcaggcatggctctgctaacactttattaaaagcatggattaattttacttccaagtttatttttactgcaccatcccatttgtggaaacaactagcttactcagcttttttttccttttataaaggaaagaacagaaaagtaaaaggaggaaagaaaacaagaggtgagtgaggcaactgaaaactgttcttggacctgcggtgctatagagcaggctcttctaggttggcagttgccatggaatctggacccaaaatgttggcccccgtttgcctggtggaaaataacaatgagcagctattggtgaaccagcaagctatacagattcttgaaaagatttctcagccagtggtggtggtggccattgtaggactgtaccgtacagggaaatcctacttgatgaaccatctggcaggacagaatcatggcttccctctgggctccacggtgcagtctgaaaccaagggcatctggatgtggtgcgtgccccacccatccaagccaaaccacaccctggtccttctggacaccgaaggtctgggcgatgtggaaaagggtgaccctaagaatgactcctggatctttgccctggctgtgctcctgtgcagcacctttgtctacaacagcatgagcaccatcaaccaccaggccctggagcagctgcattatgtgacggagctcacagaactaattaaggcaaagtcctccccaaggcctgatggagtagaagattccacagagtttgtgagtttcttcccagactttctttggacagtacgggatttcactctggagctgaagttgaacggtcaccctatcacagaagatgaatacctggagaatgccttgaagctgattcaaggcaataatcccagagttcaaacatccaattttcccagggagtgcatcaggcgtttctttccaaaacggaagtgtttcgtctttgaccggccaacaaatgacaaagaccttctagccaatattgagaaggtgtcagaaaagcaactggatcccaaattccaggaacaaacaaacattttctgttcttacatcttcactcatgcaagaaccaagaccctcagggagggaatcacagtcactgggaatcgtctgggaactctggcagtgacttatgtagaggccatcaacagtggagcagtgccttgtctggagaatgcagtgataactctggcccagcgtgagaactcagcggccgtgcagagggcagctgactactacagccagcagatggcccagcgagtgaagctccccacagacacgctccaggagctgctggacatgcatgcggcctgtgagagggaagccattgcaatcttcatggagcactccttcaaggatgaaaatcaggaattccagaagaagttcatggaaaccacaatgaataagaagggggatttcttgctgcagaatgaagagtcatctgttcaatactgccaggctaaactcaatgagctctcaaagggactaatggaaagtatctcagcaggaagtttctctgttcctggagggcacaagctctacatggaaacaaaggaaaggattgaacaggactattggcaagttcccaggaaaggagtaaaggcaaaagaggtcttccagaggttcctggagtcacagatggtgatagaggaatccatcttgcagtcagataaagccctcactgatagagagaaggcagtagcagtggatcgggccaagaaggaggcagctgagaaggaacaggaacttttaaaacagaaattacaggagcagcagcaacagatggaggctcaagataagagtcgcaaggaaaacatagcccaactgaaggagaagctgcagatggagagagaacacctactgagagagcagattatgatgttggagcacacgcagaaggtccaaaatgattggcttcatgaaggatttaagaagaagtatgaggagatgaatgcagagataagtcaatttaaacgtatgattgatactacaaaaaatgatgatactccctggattgcacgaaccttggacaaccttgccgatgagctaactgcaatattgtctgctcctgctaaattaattggtcatggtgtcaaaggtgtgagctcactctttaaaaagcataagctccccttttaaggatattatagattgtacatatatgctttggactatttttgatctgtatgtttttcattttcattcagcaagttttttttttttttcagagtcttactctgttgcccaggctggagtacagtggtgcaatctcagctcactgcaacctctgcctcctgggttcaagagattcacctgcctcagccccctagtagctgggattataggtgtacaccaccacacccagctaatttttgtatttttagtagagatggggtttcactatgttggccaggctggtctcgaactcttgacctcaaatgatccacccgcctcggcctcccaaagtgctgggtttacaggcatgagccaccatgcccagccctcatttagcaaagttttaaacataaaaagtgcttattagaggatatcagtgcctggcccacatgagagaacagatccatacacactttgaaaaactttgttcacttttaggaaatataattttgaaaaatcatttacatacaagaggtccactgaggcattgcttttaatggcaaaatattgcaatgtacttgaatgtccttcacattagattggtaagataaattttagtatgtgcatgtactggaatattatatagccagtaaacaaattgacaatgaagctctatttgtaccagtaaagaatggtcttgaagagacattgtaaaatgaaaaaaaaaaaaaccaagttgtaaagcaatgtagattatcttatcagcatggaaaaaatgcaattattatatggaagcatgcaaatatatctctggaaagattaatcaaaatctattattattggctccttctgggaagaaccaagtgaatgtaggggttgaaggaacacatactcttcactttatactcttgaattttgtaaagaaatcttattttaccgattcagaaataaataagaaaatgtaagagacaatgctaataatgataacaataaaaaaatagtgaaattggctatcagatgatgaaattccttgttgcttctaggaattacatcagccagcccaatgaggggtctaagactaagatctgagtactagaatgcaaaaaatttgcatcatattcttgttttcattttggcctggtttttcgatcccttctatttgtttcgagactaaggcatacacttttctgtggcctttgaaatctcagctacagtagcctctacttcctttctctgaattcaagttttaaggtgttagaagcataaattaccaaacattaagattaagcactgataggaaggatcctgccaaccttgtcagtagtggtctttttgcggtagcctcagatagaaaggacaatgtgcctttccagaactgaaccccctgatgggggatgatagacccagacacatagaggcctggtgttgacccttaattgttagaatctaagaagattcagttatagtaatgagtggcagactgcaaggcctgatggagataattctgaagatggtcaatacaatggaccagccatagggttaaatgggatggggagaaaacaatcaagtgaagtaaatgctgtctatcaggatgttgagaggtagccctgataaagtgtcacatccatagccttgatctgagctaatttcagaccctgaactcagttggagtggagggcagtaccccaggaaaggacactgcaactccaggacccaaataaatgataatgatttcccaagttctcctcctaggaaacgtctggagccatttactagatatccatatactagggaaatacaaacacccaaatatttcaaggacccttggaatcaggatccaagttgcccatggtacctagatcatggagagtcatcgtaaaatgcctgttaaaatagggaattagggcctccaggttgtaattatagtcttggcacacatgaacttattgcaatatagtatttgtttgaaatgtaaaggtaaagtttgtttgaagacagataccttatttttctgtacatctccttgggagtatgttcaggcataggttgtgctaaataagcttcttttataaaaacaataggctgatgcatgggtgattattttcaattgataaaatatcatgaactttccagagatcagtggtaataagttgtttgaattttcgacaccagattaaggtctctgaggtggtttaaaggctatgaggccaggtgcagtggctcacgcctataatctcagcaacttgggaggccgaagcaggcagatcacttgaggtcagaagtttgagaccagcctggccaacatggcgaaaccctgcctttactaaaaatacaaaaattaaccaagtgtggtggcacacgcctgtcatgccagctacacaggaggatgaggcaggaaaatcacttgaacctgggaggtggaggttgcagtgagccgagattgtgccattgcactccagcccgggcaacagagcctgggcgacagagtgaggctctgtctcaaaaaattaaactaaattaaagctatgagaagaaaataaattttcctggtatagtgtccttctctagacctcatgtcctacagtctctgtgaaactgggccaaagatagaatttttttttctgtgctttatacagtgactcagacaaacctatgacctcacacaaatctgtaacttcatgtatcacagtctctataaaactatatcaaagaaag

SEQ ID NO: 5 - DNA, 4526 bp, Artificial Sequence
  Human GBP6 transcript variant 2 mRNA sequence
acccaacacccatcctcaaactcacatcggatgattcaggcatggctctgctaacactttattaaaagcatggattaattttacttccaagtttatttttactgcaccatcccatttgtggaaacaactagcttactcagcttttttttccttttataaaggaaagaacagaaaagtaaaaggaggaaagaaaacaagaggtgagtgaggcaactgaaaactgttcttggacctgcggtgctatagagcagggtgaccctaagaatgactcctggatctttgccctggctgtgctcctgtgcagcacctttgtctacaacagcatgagcaccatcaaccaccaggccctggagcagctgcattatgtgacggagctcacagaactaattaaggcaaagtcctccccaaggcctgatggagtagaagattccacagagtttgtgagtttcttcccagactttctttggacagtacgggatttcactctggagctgaagttgaacggtcaccctatcacagaagatgaatacctggagaatgccttgaagctgattcaaggcaataatcccagagttcaaacatccaattttcccagggagtgcatcaggcgtttctttccaaaacggaagtgtttcgtctttgaccggccaacaaatgacaaagaccttctagccaatattgagaaggtgtcagaaaagcaactggatcccaaattccaggaacaaacaaacattttctgttcttacatcttcactcatgcaagaaccaagaccctcagggagggaatcacagtcactgggaatcgtctgggaactctggcagtgacttatgtagaggccatcaacagtggagcagtgccttgtctggagaatgcagtgataactctggcccagcgtgagaactcagcggccgtgcagagggcagctgactactacagccagcagatggcccagcgagtgaagctccccacagacacgctccaggagctgctggacatgcatgcggcctgtgagagggaagccattgcaatcttcatggagcactccttcaaggatgaaaatcaggaattccagaagaagttcatggaaaccacaatgaataagaagggggatttcttgctgcagaatgaagagtcatctgttcaatactgccaggctaaactcaatgagctctcaaagggactaatggaaagtatctcagcaggaagtttctctgttcctggagggcacaagctctacatggaaacaaaggaaaggattgaacaggactattggcaagttcccaggaaaggagtaaaggcaaaagaggtcttccagaggttcctggagtcacagatggtgatagaggaatccatcttgcagtcagataaagccctcactgatagagagaaggcagtagcagtggatcgggccaagaaggaggcagctgagaaggaacaggaacttttaaaacagaaattacaggagcagcagcaacagatggaggctcaagataagagtcgcaaggaaaacatagcccaactgaaggagaagctgcagatggagagagaacacctactgagagagcagattatgatgttggagcacacgcagaaggtccaaaatgattggcttcatgaaggatttaagaagaagtatgaggagatgaatgcagagataagtcaatttaaacgtatgattgatactacaaaaaatgatgatactccctggattgcacgaaccttggacaaccttgccgatgagctaactgcaatattgtctgctcctgctaaattaattggtcatggtgtcaaaggtgtgagctcactctttaaaaagcataagctccccttttaaggatattatagattgtacatatatgctttggactatttttgatctgtatgtttttcattttcattcagcaagttttttttttttttcagagtcttactctgttgcccaggctggagtacagtggtgcaatctcagctcactgcaacctctgcctcctgggttcaagagattcacctgcctcagccccctagtagctgggattataggtgtacaccaccacacccagctaatttttgtatttttagtagagatggggtttcactatgttggccaggctggtctcgaactcttgacctcaaatgatccacccgcctcggcctcccaaagtgctgggtttacaggcatgagccaccatgcccagccctcatttagcaaagttttaaacataaaaagtgcttattagaggatatcagtgcctggcccacatgagagaacagatccatacacactttgaaaaactttgttcacttttaggaaatataattttgaaaaatcatttacatacaagaggtccactgaggcattgcttttaatggcaaaatattgcaatgtacttgaatgtccttcacattagattggtaagataaattttagtatgtgcatgtactggaatattatatagccagtaaacaaattgacaatgaagctctatttgtaccagtaaagaatggtcttgaagagacattgtaaaatgaaaaaaaaaaaaaccaagttgtaaagcaatgtagattatcttatcagcatggaaaaaatgcaattattatatggaagcatgcaaatatatctctggaaagattaatcaaaatctattattattggctccttctgggaagaaccaagtgaatgtaggggttgaaggaacacatactcttcactttatactcttgaattttgtaaagaaatcttattttaccgattcagaaataaataagaaaatgtaagagacaatgctaataatgataacaataaaaaaatagtgaaattggctatcagatgatgaaattccttgttgcttctaggaattacatcagccagcccaatgaggggtctaagactaagatctgagtactagaatgcaaaaaatttgcatcatattcttgttttcattttggcctggtttttcgatcccttctatttgtttcgagactaaggcatacacttttctgtggcctttgaaatctcagctacagtagcctctacttcctttctctgaattcaagttttaaggtgttagaagcataaattaccaaacattaagattaagcactgataggaaggatcctgccaaccttgtcagtagtggtctttttgcggtagcctcagatagaaaggacaatgtgcctttccagaactgaaccccctgatgggggatgatagacccagacacatagaggcctggtgttgacccttaattgttagaatctaagaagattcagttatagtaatgagtggcagactgcaaggcctgatggagataattctgaagatggtcaatacaatggaccagccatagggttaaatgggatggggagaaaacaatcaagtgaagtaaatgctgtctatcaggatgttgagaggtagccctgataaagtgtcacatccatagccttgatctgagctaatttcagaccctgaactcagttggagtggagggcagtaccccaggaaaggacactgcaactccaggacccaaataaatgataatgatttcccaagttctcctcctaggaaacgtctggagccatttactagatatccatatactagggaaatacaaacacccaaatatttcaaggacccttggaatcaggatccaagttgcccatggtacctagatcatggagagtcatcgtaaaatgcctgttaaaatagggaattagggcctccaggttgtaattatagtcttggcacacatgaacttattgcaatatagtatttgtttgaaatgtaaaggtaaagtttgtttgaagacagataccttatttttctgtacatctccttgggagtatgttcaggcataggttgtgctaaataagcttcttttataaaaacaataggctgatgcatgggtgattattttcaattgataaaatatcatgaactttccagagatcagtggtaataagttgtttgaattttcgacaccagattaaggtctctgaggtggtttaaaggctatgaggccaggtgcagtggctcacgcctataatctcagcaacttgggaggccgaagcaggcagatcacttgaggtcagaagtttgagaccagcctggccaacatggcgaaaccctgcctttactaaaaatacaaaaattaaccaagtgtggtggcacacgcctgtcatgccagctacacaggaggatgaggcaggaaaatcacttgaacctgggaggtggaggttgcagtgagccgagattgtgccattgcactccagcccgggcaacagagcctgggcgacagagtgaggctctgtctcaaaaaattaaactaaattaaagctatgagaagaaaataaattttcctggtatagtgtccttctctagacctcatgtcctacagtctctgtgaaactgggccaaagatagaatttttttttctgtgctttatacagtgactcagacaaacctatgacctcacacaaatctgtaacttcatgtatcacagtctctataaaactatatcaaagaaag

SEQ ID NO: 6 - DNA, 5496 bp, Artificial Sequence
  Human TMCC1 transcript variant 3 mRNA sequence
ggcggccgcagtggaaggagcaggcgcttgagctcgagcgacggcgctggcggagacgccggctgctcctcccctccccgccgcttttcctaaaaggattgtacaccttagaagtgcttaaggaagagtgatgaagctctgaatcgtgtcctgcagcagattcgagtgccacccaagatgaagagagggacaagcttgcatagtaggcggggcaagccagaggccccaaagggaagtccccaaatcaacaggaagtctggtcaggagatgacagctgttatgcagtcaggccgacccaggtcttcatccacaactgatgcacctaccagctctgctatgatggaaatagcttgtgctgctgctgctgctgctgctgcatgtctaccaggagaggagggaactgcggagcggatcgaacggttggaagtaagcagccttgcccaaacatccagtgcagtggcctccagtaccgatggcagcatccacacagactctgtggatggaacaccagaccctcagcgcacaaaggctgccattgctcacctgcagcagaagatcctgaagctcacagaacaaatcaagattgcacaaacagcccgggacgacaacgttgctgaatacttgaagcttgccaacagtgcagacaaacagcaggctgcccgcatcaagcaagtctttgagaagaagaaccagaaatctgcccaaactatcctccagctgcaaaagaaacttgagcactaccacaggaagctcagagaggtagagcagaatgggatcccccggcagccaaaggatgtcttcagggacatgcaccagggtctgaaggatgtaggagcaaaggtgactggcttcagtgaaggtgtggtggatagtgtcaaaggtgggttttccagcttctcccaggccacccattcagcagcaggcgctgtagtctcaaagcccagagagattgcctcactcattcggaacaaatttggcagtgcagacaacatccccaacctgaaggactctttagaggaagggcaagtggatgatgcggggaaggctttgggagtgatttcaaactttcagtctagcccaaaatatggtagtgaagaagattgttctagtgccacttcaggctcagtgggagccaacagcaccacagggggcatcgctgtaggagcatccagctccaaaacaaacaccctggacatgcagagctcaggatttgatgcactactacatgagatccaggagatccgggaaacccaggccagactagaggaatcctttgagactctcaaggaacattatcagagggactattccttaataatgcagaccttacaggaggagcgatatagatgtgaacgattggaagaacagctaaatgacctaacagagctccaccagaatgaaatcttgaacttgaagcaggaactggcaagcatggaagaaaaaatcgcgtatcagtcctatgaacgggcccgggacatccaggaggccctggaggcatgccagacgcgcatctccaagatggagctgcagcagcagcagcagcaggtggtgcagctagaagggctggagaatgccactgcccggaaccttctgggcaaactcatcaacatcctcctggctgtcatggcagtccttttggtctttgtctccactgtagccaactgtgtggtccccctcatgaagactcgcaacaggacgttcagcactttattccttgtggtttttattgcctttctctggaagcactgggacgccctcttcagctatgtggaacggttcttttcatcccctagatgatgctggcacagaaggcattgttccctaccctctggcgagtgcatgcagcagagagttagacagcaacttacctactctgaagttttctacaacaaaaaaagagttgagtgaatctgtttacatttagaataatgtttttttcttcaagagacgcaattgcaatagtattttttagattttatccaagaagttttttgggcgaaaatcttggatcatttttatgtagcatgattttccttgggatgcaaatcttaaaacagtcctttaatatgaaccaacaatctggagcacaccgaagggcaatctaaattgtggcttgaaggactgcactaaaacccactaaaaagatgcgaaaacctgatgagggcaaaccagttaaacctaacaccctgccttgtctgggctcatcacctctccctatcccagactaactttactgtgaaatcctaccacattccatgtctgaatttttggattcggggtggattttcgttgtccgtggaagaacacatggatctctctggctttctcacccaagttggccacttacgctaatcctggaagtatgatcacttttgaacctgccccttaaccttgacgaggatacaaaagtgaaagcatcatcccccaaaggatcactgcacagtcctactacagtatttttaagtagccctctaaatacttaattttaagcaaaatcccttggccgcacttttaaggtttttttatatgtgtatagttaccaacctaaaaataaaaaatccgaacagcatacttgaagaatgtaatactcaaactctcagtgcttccttatggtttctaataggattttttattattgttattattattattgggtttttttggacagggttgggagggtcttttatttttcctttgaaataaagaagtgatgtttttaaatgaagaaatgtgtggatatttaagtgtgctgctccctcttgtcttgaaacagtttgagtaagaaagtcttgctgtaaatgctgccctctgccgcctttgttttgagatgcagtttaaactccctctggctgctgctgctgctttttggtgtcccgacatacctacgcccccgttttatgggtttggcttagttgaagaggaaagggttgtgcaaggagagcaggaggctgtttccaaaaaccagtgtagtaggatagggattttttttttttttttttgccccaagaaaacgttcacccagtgatcttgggctggggttgtctttaggaaaagttgagactataagagtcataaataagtccttgtgtttccttaatttattttgttaacacccctaattacaaccaaagtgatgatgtggagtcttctgtcttcattttggccccagcattcttaatttcaaagctttattctgtctgcctaagagaatcaaccaaaggtgattctcctaaagagcagtgaaggaaatgtcaggttagcaggacccaagttttgggtgtgaaatgttgccagcttcctataatgtaaacggacttgttaacctaacctaattatgctcagtggacttctatagatggttttgaaaaatgaactgagctgccttcccccgtcgcataaccagttccatcatcctggtggaacttgaacatttagagtttatctagagagcttggttaatctttccatattatttgtagtattggtcacaaatgctgttccctcttagcctcattctgtgcaaccaagtgcatataagatgccctgaaaagagtaacaaagtatgctttgcctgtttccacttaccaggaaattccttcagaactagattagcattgccctgcctgtctgaaaggacagtttacctaatggtgccagcctccttttgctttggcaagctggatttctcagagccagcatgttgtttccataactactttgatattttaactcaggtactccagtcttcaccccaacctcagctgattgtagtacacctgctagctctgttgccccctcaaaactgcacccagagcagggccacaagggtgctttttttctttaaaaaaaaaaaaattagaaccaattcatgttcatgccaaaaacaaattgtccccaagcctatatgtattaaaatgttaactttgcctaaaaatattgcagtgactttttaggcaggagtgccaaaggacactatgaactttttgaactgacagtttctcctaactttctgctttagcgtaattgctcagagtagagagcccccacaaagttatttaaaagatgccctagcagcaatccaccagtttttctaagctagaacctttgagtcccccaaactgcctgaagacttaagttttgtgggcactggaagtcactttgatagatggattgaaactgttcctatttgccctgggacggtttctatctatcaaaggaaggttttcacctgtagaaagccccctgcctccagccaaatagtcccatgctgactttctatcttcctttctcaaactgtcttaggaaggaccttcagtgcagatcaggtgcagtaatggctttcttgtcccttaattattcaccagacccagaagttgtacgcatttaatgctgtttgtaaccatgcatctgttttcattctttgctgtaccttttgctgcccatcctgttacttttgagtttctttcattgtggttgttcttgggttcttttgtcttgtcagagctcttctataacctcgctctaatggcttaacagttgttctgggtggaaacgtcccctcatttgaatgctcctctaaaaaaaaaagaaaagaaaactgtattcattccctttaaaatgaaacattcctggtttatttgtccatgcctctagcctgggtgagtgaagctggctgtgttggcctgggtgattgttcaggttgtagaatgcgcattttacattgtttatgataattgaagggtacttctgtctgctccaatttccattccttggtatactcagtatgtcctagtaaggaaggctttccactctactggctccttaagaagcaaaagtaggttaaattttatacttcactgactagggtcttctctctcccctgttctgaatggagtaaaagtctgatgccaagacaaatattgggagcgagccttcctcactagccatgtccaaataatgagcgtattttcatgtggtcttcactgcatttggttttgtttctgatttcatgttcctttgaggtacagtcagatgaaaatgctgagttctgagagagttccaatgaggagctgcctttcagctttggaaaatatgcaactaaaacaaaaccaaacattattgtaatctgacacaggcaaaattatggttcccacaccccaaccccaaatgaaacctgggattttgaatgtggctctaagagttaacattgctgtctgtatgtcgtgtatgctaggtgatatcctcagtagggattgactactagactgtgtgttttatcaaagtgtgtaagaataaaaactcacttgcacgaattgaaacgtaaagaaatgacacttgtgaacgtgtgaacgttaacactgtagttgatagatcttaagctgctaattgttgagagagatttaaatttatgttctgtctggtcgctgcagattctcagcagcacttttactgtatatagaaattgaaataaagacctcagctattaaaaaaaaaaaa

SEQ ID NO: 7 - DNA, 6095 bp, Artificial Sequence
  Human TMCC1 transcript variant 1 mRNA sequence
gtattaatctctggagaagacacatccacagttagcactttcttcagatgctgacgctcggtgaacagttgcctttggtcacaagatttagaagacacagtgtccatcctcccagattggatctctttttcatatggatcttctgtttctatgtctttttaaaaaataactttttgggaaaccttttggattacaactgttcatcctcacctatgcaaagaaagggaagctattgctgggattttgaggagcttttcctaaaaggattgtacaccttagaagtgcttaaggaagagtgatgaagataggcatgaagccttcgtctcacagctgcatgcgtagtcactgttgaagcaaatgcctacctaatttgacactcttggtgtgtttaaaaaatttttttgagtttgcaaataagcatattaagtctactgatggagccttcgggcagtgaacagttatttgaggaccctgatcctggaggcaaatcccaagatgcagaggccagaaagcagacagaatcagaacaaaaattgtctaaaatgacccacaatgctttggagaacattaacgtgattggccaaggcttgaagcatctcttccagcaccagcgcaggaggtcatcagtgtctccacatgatgtgcagcaaattcaggcagatccagaacctgaaatggatctggaaagccagaacgcatgtgctgagattgatggtgtccccacccaccccacagctctgaatcgtgtcctgcagcagattcgagtgccacccaagatgaagagagggacaagcttgcatagtaggcggggcaagccagaggccccaaagggaagtccccaaatcaacaggaagtctggtcaggagatgacagctgttatgcagtcaggccgacccaggtcttcatccacaactgatgcacctaccagctctgctatgatggaaatagcttgtgctgctgctgctgctgctgctgcatgtctaccaggagaggagggaactgcggagcggatcgaacggttggaagtaagcagccttgcccaaacatccagtgcagtggcctccagtaccgatggcagcatccacacagactctgtggatggaacaccagaccctcagcgcacaaaggctgccattgctcacctgcagcagaagatcctgaagctcacagaacaaatcaagattgcacaaacagcccgggacgacaacgttgctgaatacttgaagcttgccaacagtgcagacaaacagcaggctgcccgcatcaagcaagtctttgagaagaagaaccagaaatctgcccaaactatcctccagctgcaaaagaaacttgagcactaccacaggaagctcagagaggtagagcagaatgggatcccccggcagccaaaggatgtcttcagggacatgcaccagggtctgaaggatgtaggagcaaaggtgactggcttcagtgaaggtgtggtggatagtgtcaaaggtgggttttccagcttctcccaggccacccattcagcagcaggcgctgtagtctcaaagcccagagagattgcctcactcattcggaacaaatttggcagtgcagacaacatccccaacctgaaggactctttagaggaagggcaagtggatgatgcggggaaggctttgggagtgatttcaaactttcagtctagcccaaaatatggtagtgaagaagattgttctagtgccacttcaggctcagtgggagccaacagcaccacagggggcatcgctgtaggagcatccagctccaaaacaaacaccctggacatgcagagctcaggatttgatgcactactacatgagatccaggagatccgggaaacccaggccagactagaggaatcctttgagactctcaaggaacattatcagagggactattccttaataatgcagaccttacaggaggagcgatatagatgtgaacgattggaagaacagctaaatgacctaacagagctccaccagaatgaaatcttgaacttgaagcaggaactggcaagcatggaagaaaaaatcgcgtatcagtcctatgaacgggcccgggacatccaggaggccctggaggcatgccagacgcgcatctccaagatggagctgcagcagcagcagcagcaggtggtgcagctagaagggctggagaatgccactgcccggaaccttctgggcaaactcatcaacatcctcctggctgtcatggcagtccttttggtctttgtctccactgtagccaactgtgtggtccccctcatgaagactcgcaacaggacgttcagcactttattccttgtggtttttattgcctttctctggaagcactgggacgccctcttcagctatgtggaacggttcttttcatcccctagatgatgctggcacagaaggcattgttccctaccctctggcgagtgcatgcagcagagagttagacagcaacttacctactctgaagttttctacaacaaaaaaagagttgagtgaatctgtttacatttagaataatgtttttttcttcaagagacgcaattgcaatagtattttttagattttatccaagaagttttttgggcgaaaatcttggatcatttttatgtagcatgattttccttgggatgcaaatcttaaaacagtcctttaatatgaaccaacaatctggagcacaccgaagggcaatctaaattgtggcttgaaggactgcactaaaacccactaaaaagatgcgaaaacctgatgagggcaaaccagttaaacctaacaccctgccttgtctgggctcatcacctctccctatcccagactaactttactgtgaaatcctaccacattccatgtctgaatttttggattcggggtggattttcgttgtccgtggaagaacacatggatctctctggctttctcacccaagttggccacttacgctaatcctggaagtatgatcacttttgaacctgccccttaaccttgacgaggatacaaaagtgaaagcatcatcccccaaaggatcactgcacagtcctactacagtatttttaagtagccctctaaatacttaattttaagcaaaatcccttggccgcacttttaaggtttttttatatgtgtatagttaccaacctaaaaataaaaaatccgaacagcatacttgaagaatgtaatactcaaactctcagtgcttccttatggtttctaataggattttttattattgttattattattattgggtttttttggacagggttgggagggtcttttatttttcctttgaaataaagaagtgatgtttttaaatgaagaaatgtgtggatatttaagtgtgctgctccctcttgtcttgaaacagtttgagtaagaaagtcttgctgtaaatgctgccctctgccgcctttgttttgagatgcagtttaaactccctctggctgctgctgctgctttttggtgtcccgacatacctacgcccccgttttatgggtttggcttagttgaagaggaaagggttgtgcaaggagagcaggaggctgtttccaaaaaccagtgtagtaggatagggattttttttttttttttttgccccaagaaaacgttcacccagtgatcttgggctggggttgtctttaggaaaagttgagactataagagtcataaataagtccttgtgtttccttaatttattttgttaacacccctaattacaaccaaagtgatgatgtggagtcttctgtcttcattttggccccagcattcttaatttcaaagctttattctgtctgcctaagagaatcaaccaaaggtgattctcctaaagagcagtgaaggaaatgtcaggttagcaggacccaagttttgggtgtgaaatgttgccagcttcctataatgtaaacggacttgttaacctaacctaattatgctcagtggacttctatagatggttttgaaaaatgaactgagctgccttcccccgtcgcataaccagttccatcatcctggtggaacttgaacatttagagtttatctagagagcttggttaatctttccatattatttgtagtattggtcacaaatgctgttccctcttagcctcattctgtgcaaccaagtgcatataagatgccctgaaaagagtaacaaagtatgctttgcctgtttccacttaccaggaaattccttcagaactagattagcattgccctgcctgtctgaaaggacagtttacctaatggtgccagcctccttttgctttggcaagctggatttctcagagccagcatgttgtttccataactactttgatattttaactcaggtactccagtcttcaccccaacctcagctgattgtagtacacctgctagctctgttgccccctcaaaactgcacccagagcagggccacaagggtgctttttttctttaaaaaaaaaaaaattagaaccaattcatgttcatgccaaaaacaaattgtccccaagcctatatgtattaaaatgttaactttgcctaaaaatattgcagtgactttttaggcaggagtgccaaaggacactatgaactttttgaactgacagtttctcctaactttctgctttagcgtaattgctcagagtagagagcccccacaaagttatttaaaagatgccctagcagcaatccaccagtttttctaagctagaacctttgagtcccccaaactgcctgaagacttaagttttgtgggcactggaagtcactttgatagatggattgaaactgttcctatttgccctgggacggtttctatctatcaaaggaaggttttcacctgtagaaagccccctgcctccagccaaatagtcccatgctgactttctatcttcctttctcaaactgtcttaggaaggaccttcagtgcagatcaggtgcagtaatggctttcttgtcccttaattattcaccagacccagaagttgtacgcatttaatgctgtttgtaaccatgcatctgttttcattctttgctgtaccttttgctgcccatcctgttacttttgagtttctttcattgtggttgttcttgggttcttttgtcttgtcagagctcttctataacctcgctctaatggcttaacagttgttctgggtggaaacgtcccctcatttgaatgctcctctaaaaaaaaaagaaaagaaaactgtattcattccctttaaaatgaaacattcctggtttatttgtccatgcctctagcctgggtgagtgaagctggctgtgttggcctgggtgattgttcaggttgtagaatgcgcattttacattgtttatgataattgaagggtacttctgtctgctccaatttccattccttggtatactcagtatgtcctagtaaggaaggctttccactctactggctccttaagaagcaaaagtaggttaaattttatacttcactgactagggtcttctctctcccctgttctgaatggagtaaaagtctgatgccaagacaaatattgggagcgagccttcctcactagccatgtccaaataatgagcgtattttcatgtggtcttcactgcatttggttttgtttctgatttcatgttcctttgaggtacagtcagatgaaaatgctgagttctgagagagttccaatgaggagctgcctttcagctttggaaaatatgcaactaaaacaaaaccaaacattattgtaatctgacacaggcaaaattatggttcccacaccccaaccccaaatgaaacctgggattttgaatgtggctctaagagttaacattgctgtctgtatgtcgtgtatgctaggtgatatcctcagtagggattgactactagactgtgtgttttatcaaagtgtgtaagaataaaaactcacttgcacgaattgaaacgtaaagaaatgacacttgtgaacgtgtgaacgttaacactgtagttgatagatcttaagctgctaattgttgagagagatttaaatttatgttctgtctggtcgctgcagattctcagcagcacttttactgtatatagaaattgaaataaagacctcagctattaaaaaaaaaaaa

SEQ ID NO: 8 - DNA, 4753 bp, Artificial Sequence
  Human PRDM1 transcript variant 2 mRNA sequence
agtttgacgtcgtcagccggcttggtcttctacccagtgactcaaagcactaaaagtcagcataatcggaactgaagtcagtagcatcgcccatttgccattcactgcagtagcaaaagtagtactctgtggtgggttaatcggtttgaggcagctccttaaatgaacatttgtgtttcatttttctgttattttcccgaacatgaaaagacgataaaactgaaatggaaaagatctattccagaggggagcttcaccacttcattgacggctttaatgaagagaaaagcaactggatgcgctatgtgaatccagcacactctccccgggagcaaaacctggctgcgtgtcagaacgggatgaacatctacttctacaccattaagcccatccctgccaaccaggaacttcttgtgtggtattgtcgggactttgcagaaaggcttcactacccttatcccggagagctgacaatgatgaatctcacacaaacacagagcagtctaaagcaaccgagcactgagaaaaatgaactctgcccaaagaatgtcccaaagagagagtacagcgtgaaagaaatcctaaaattggactccaacccctccaaaggaaaggacctctaccgttctaacatttcacccctcacatcagaaaaggacctcgatgactttagaagacgtgggagccccgaaatgcccttctaccctcgggtcgtttaccccatccgggcccctctgccagaagactttttgaaagcttccctggcctacgggatcgagagacccacgtacatcactcgctcccccattccatcctccaccactccaagcccctctgcaagaagcagccccgaccaaagcctcaagagctccagccctcacagcagccctgggaatacggtgtcccctgtgggccccggctctcaagagcaccgggactcctacgcttacttgaacgcgtcctacggcacggaaggtttgggctcctaccctggctacgcacccctgccccacctcccgccagctttcatcccctcgtacaacgctcactaccccaagttcctcttgcccccctacggcatgaattgtaatggcctgagcgctgtgagcagcatgaatggcatcaacaactttggcctcttcccgaggctgtgccctgtctacagcaatctcctcggtgggggcagcctgccccaccccatgctcaaccccacttctctcccgagctcgctgccctcagatggagcccggaggttgctccagccggagcatcccagggaggtgcttgtcccggcgccccacagtgccttctcctttaccggggccgccgccagcatgaaggacaaggcctgtagccccacaagcgggtctcccacggcgggaacagccgccacggcagaacatgtggtgcagcccaaagctacctcagcagcgatggcagcccccagcagcgacgaagccatgaatctcattaaaaacaaaagaaacatgaccggctacaagacccttccctacccgctgaagaagcagaacggcaagatcaagtacgaatgcaacgtttgcgccaagactttcggccagctctccaatctgaaggtccacctgagagtgcacagtggagaacggcctttcaaatgtcagacttgcaacaagggctttactcagctcgcccacctgcagaaacactacctggtacacacgggagaaaagccacatgaatgccaggtctgccacaagagatttagcagcaccagcaatctcaagacccacctgcgactccattctggagagaaaccataccaatgcaaggtgtgccctgccaagttcacccagtttgtgcacctgaaactgcacaagcgtctgcacacccgggagcggccccacaagtgctcccagtgccacaagaactacatccatctctgtagcctcaaggttcacctgaaagggaactgcgctgcggccccggcgcctgggctgcccttggaagatctgacccgaatcaatgaagaaatcgagaagtttgacatcagtgacaatgctgaccggctcgaggacgtggaggatgacatcagtgtgatctctgtagtggagaaggaaattctggccgtggtcagaaaagagaaagaagaaactggcctgaaagtgtctttgcaaagaaacatggggaatggactcctctcctcagggtgcagcctttatgagtcatcagatctacccctcatgaagttgcctcccagcaacccactacctctggtacctgtaaaggtcaaacaagaaacagttgaaccaatggatccttaagattttcagaaaacacttattttgtttcttaagttatgacttggtgagtcagggtgcctgtaggaagtggcttgtacataatcccagctctgcaaagctctctcgacagcaaatggtttcccctcacctctggaattaaagaaggaactccaaagttactgaaatctcagggcatgaacaaggcaaaggccatatatatatatatatatatatctgtatacatattatatatacttatttacacctgtgtctatatatttgcccctgtgtattttgaatatttgtgtggacatgtttgcatagccttcccattactaagactattacctagtcataattattttttcaatgataatccttcataatttattatacaatttatcattcagaaagcaataattaaaaaagtttacaatgactggaaagattccttgtaatttgagtataaatgtatttttgtcttgtggccattctttgtagataatttctgcacatctgtataagtacctaagatttagttaaacaaatatatgacttcagtcaacctctctctctaataatggtttgaaaatgaggtttgggtaattgccaatgttggacagttgatgtgttcattcctgggatcctatcatttgaacagcattgtacataacttgggggtatgtgtgcaggattacccaagaataacttaagtagaagaaacaagaaagggaatcttgtatatttttgttgatagttcatgtttttcccccagccacaattttaccggaagggtgacaggaaggctttaccaacctgtctctccctccaaaagagcagaatcctcccaccgccctgccctccccaccgagtcctgtggccattcagagcggccacatgacttttgcatccattgtattatcagaaaatgtgaagaagaaaaaaatgccatgttttaaaaccactgcgaaaatttccccaaagcataggtggctttgtgtgtgtgcgatttgggggcttgagtctgggtggtgttttgttgttggtttttgttgctttttttttttttttttttttaatgtcaaaattgcacaaacatggtgctctaccaggaaggattcgaggtagataggctcaggccacactttaaaaacaaacacacaaacaacaaaaaacgggtattctagtcatcttggggtaaaagcgggtaatgaacattcctatccccaacacatcaattgtattttttctgtaaaactcagattttcctcagtatttgtgtttttacattttatggttaatttaatggaagatgaaagggcattgcaaagttgttcaacaacagttacctcattgagtgtgtccagtagtgcaggaaatgatgtcttatctaatgatttgcttctctagaggagaaaccgagtaaatgtgctccagcaagatagactttgtgttattctatcttttattctgctaagcccaaagattacatgttggtgttcaaagtgtagcaaaaaatgatgtatatttataaatctatttataccactatatcatatgtatatatatttataaccacttaaattgtgagccaagccatgtaaaagatctactttttctaagggcaaaaaaaaaaaaaaaaaaaaaagaacactcctttctgagactttgcttaatacttggtgacctcacaatcacgtcggtatgattgggcacccttgcctactgtaagagaccctaaaaccttggtgcagtggtggggaccacaaaacaaccagggaggaagagatacatcattttttagtattaaggaccatctaagacagctctattttttttttgccactttatgattatgtggtcacacccaagtcacagaaataaaaaactgactttaccgctgcaatttttctgttttcctccttactaaatactgatacattactccaatctattttataattatatttgacattttgttcacatcaactaatgttcacctgtagaagagaacaaatttcgaataatccagggaaacccaagagccttactggtcttctgtaacttccaagactgacagctttttatgtatcagtgtttgataaacacagtccttaactgaaggtaaaccaaagcatcacgttgacattagaccaaatacttttgattcccaactactcgtttgttctttttctccttttgtgctttcccatagtgagaatttttataaagacttcttgcttctctcaccatccatccttctcttttctgcctcttacatgtgaatgttgagcccacaatcaacagtggttttattttttcctctactcaaagttaaaactgaccaaagttactggctttttactttgctagaacaacaaactatcttatgtttacatactggtttacaatgttatttatgtgcaaattgtcaaaatgtaaattaaatataaatgttcatgctttaccaaaat

SEQ ID NO: 9 - DNA, 5165 bp, Artificial Sequence
  Human PRDM1 transcript variant 1 mRNA sequence
gggaagccagacggttaacacagacaaagtgctgccgtgacactcggccctccagtgttgcggagaggcaagagcagcgaccgcggcacctgtccgcccggagctgggacgcgggcgcccgggcggccggacgaagcgaggagggaccgccgaggtgcgcgtctgtgcggctcagcctggcgggggacgcggggagaatgtggactgggtagagatgaacgagacttttctcagatgttggatatttgcttggaaaaacgtgtgggtacgaccttggctgcccccaagtgtaactccagcactgtgaggtttcagggattggcagaggggaccaaggggaccatgaaaatggacatggaggatgcggatatgactctgtggacagaggctgagtttgaagagaagtgtacatacattgtgaacgaccacccctgggattctggtgctgatggcggtacttcggttcaggcggaggcatccttaccaaggaatctgcttttcaagtatgccaccaacagtgaagaggttattggagtgatgagtaaagaatacataccaaagggcacacgttttggacccctaataggtgaaatctacaccaatgacacagttcctaagaacgccaacaggaaatatttttggaggatctattccagaggggagcttcaccacttcattgacggctttaatgaagagaaaagcaactggatgcgctatgtgaatccagcacactctccccgggagcaaaacctggctgcgtgtcagaacgggatgaacatctacttctacaccattaagcccatccctgccaaccaggaacttcttgtgtggtattgtcgggactttgcagaaaggcttcactacccttatcccggagagctgacaatgatgaatctcacacaaacacagagcagtctaaagcaaccgagcactgagaaaaatgaactctgcccaaagaatgtcccaaagagagagtacagcgtgaaagaaatcctaaaattggactccaacccctccaaaggaaaggacctctaccgttctaacatttcacccctcacatcagaaaaggacctcgatgactttagaagacgtgggagccccgaaatgcccttctaccctcgggtcgtttaccccatccgggcccctctgccagaagactttttgaaagcttccctggcctacgggatcgagagacccacgtacatcactcgctcccccattccatcctccaccactccaagcccctctgcaagaagcagccccgaccaaagcctcaagagctccagccctcacagcagccctgggaatacggtgtcccctgtgggccccggctctcaagagcaccgggactcctacgcttacttgaacgcgtcctacggcacggaaggtttgggctcctaccctggctacgcacccctgccccacctcccgccagctttcatcccctcgtacaacgctcactaccccaagttcctcttgcccccctacggcatgaattgtaatggcctgagcgctgtgagcagcatgaatggcatcaacaactttggcctcttcccgaggctgtgccctgtctacagcaatctcctcggtgggggcagcctgccccaccccatgctcaaccccacttctctcccgagctcgctgccctcagatggagcccggaggttgctccagccggagcatcccagggaggtgcttgtcccggcgccccacagtgccttctcctttaccggggccgccgccagcatgaaggacaaggcctgtagccccacaagcgggtctcccacggcgggaacagccgccacggcagaacatgtggtgcagcccaaagctacctcagcagcgatggcagcccccagcagcgacgaagccatgaatctcattaaaaacaaaagaaacatgaccggctacaagacccttccctacccgctgaagaagcagaacggcaagatcaagtacgaatgcaacgtttgcgccaagactttcggccagctctccaatctgaaggtccacctgagagtgcacagtggagaacggcctttcaaatgtcagacttgcaacaagggctttactcagctcgcccacctgcagaaacactacctggtacacacgggagaaaagccacatgaatgccaggtctgccacaagagatttagcagcaccagcaatctcaagacccacctgcgactccattctggagagaaaccataccaatgcaaggtgtgccctgccaagttcacccagtttgtgcacctgaaactgcacaagcgtctgcacacccgggagcggccccacaagtgctcccagtgccacaagaactacatccatctctgtagcctcaaggttcacctgaaagggaactgcgctgcggccccggcgcctgggctgcccttggaagatctgacccgaatcaatgaagaaatcgagaagtttgacatcagtgacaatgctgaccggctcgaggacgtggaggatgacatcagtgtgatctctgtagtggagaaggaaattctggccgtggtcagaaaagagaaagaagaaactggcctgaaagtgtctttgcaaagaaacatggggaatggactcctctcctcagggtgcagcctttatgagtcatcagatctacccctcatgaagttgcctcccagcaacccactacctctggtacctgtaaaggtcaaacaagaaacagttgaaccaatggatccttaagattttcagaaaacacttattttgtttcttaagttatgacttggtgagtcagggtgcctgtaggaagtggcttgtacataatcccagctctgcaaagctctctcgacagcaaatggtttcccctcacctctggaattaaagaaggaactccaaagttactgaaatctcagggcatgaacaaggcaaaggccatatatatatatatatatatatctgtatacatattatatatacttatttacacctgtgtctatatatttgcccctgtgtattttgaatatttgtgtggacatgtttgcatagccttcccattactaagactattacctagtcataattattttttcaatgataatccttcataatttattatacaatttatcattcagaaagcaataattaaaaaagtttacaatgactggaaagattccttgtaatttgagtataaatgtatttttgtcttgtggccattctttgtagataatttctgcacatctgtataagtacctaagatttagttaaacaaatatatgacttcagtcaacctctctctctaataatggtttgaaaatgaggtttgggtaattgccaatgttggacagttgatgtgttcattcctgggatcctatcatttgaacagcattgtacataacttgggggtatgtgtgcaggattacccaagaataacttaagtagaagaaacaagaaagggaatcttgtatatttttgttgatagttcatgtttttcccccagccacaattttaccggaagggtgacaggaaggctttaccaacctgtctctccctccaaaagagcagaatcctcccaccgccctgccctccccaccgagtcctgtggccattcagagcggccacatgacttttgcatccattgtattatcagaaaatgtgaagaagaaaaaaatgccatgttttaaaaccactgcgaaaatttccccaaagcataggtggctttgtgtgtgtgcgatttgggggcttgagtctgggtggtgttttgttgttggtttttgttgctttttttttttttttttttttaatgtcaaaattgcacaaacatggtgctctaccaggaaggattcgaggtagataggctcaggccacactttaaaaacaaacacacaaacaacaaaaaacgggtattctagtcatcttggggtaaaagcgggtaatgaacattcctatccccaacacatcaattgtattttttctgtaaaactcagattttcctcagtatttgtgtttttacattttatggttaatttaatggaagatgaaagggcattgcaaagttgttcaacaacagttacctcattgagtgtgtccagtagtgcaggaaatgatgtcttatctaatgatttgcttctctagaggagaaaccgagtaaatgtgctccagcaagatagactttgtgttattctatcttttattctgctaagcccaaagattacatgttggtgttcaaagtgtagcaaaaaatgatgtatatttataaatctatttataccactatatcatatgtatatatatttataaccacttaaattgtgagccaagccatgtaaaagatctactttttctaagggcaaaaaaaaaaaaaaaaaaaaaagaacactcctttctgagactttgcttaatacttggtgacctcacaatcacgtcggtatgattgggcacccttgcctactgtaagagaccctaaaaccttggtgcagtggtggggaccacaaaacaaccagggaggaagagatacatcattttttagtattaaggaccatctaagacagctctattttttttttgccactttatgattatgtggtcacacccaagtcacagaaataaaaaactgactttaccgctgcaatttttctgttttcctccttactaaatactgatacattactccaatctattttataattatatttgacattttgttcacatcaactaatgttcacctgtagaagagaacaaatttcgaataatccagggaaacccaagagccttactggtcttctgtaacttccaagactgacagctttttatgtatcagtgtttgataaacacagtccttaactgaaggtaaaccaaagcatcacgttgacattagaccaaatacttttgattcccaactactcgtttgttctttttctccttttgtgctttcccatagtgagaatttttataaagacttcttgcttctctcaccatccatccttctcttttctgcctcttacatgtgaatgttgagcccacaatcaacagtggttttattttttcctctactcaaagttaaaactgaccaaagttactggctttttactttgctagaacaacaaactatcttatgtttacatactggtttacaatgttatttatgtgcaaattgtcaaaatgtaaattaaatataaatgttcatgctttaccaaaat

SEQ ID NO: 10 - DNA, 1475 bp, Artificial Sequence
  Human ARG1 transcript variant 2 mRNA sequence
ggaaaaaaaagatgcgccctctgtcactgagggttgactgactggagagctcaagtgcagcaaagagaagtgtcagagcatgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaaatgtggaaacatccgatataaatctcatagttaatggcataattagaaagctaatcattttcttaagcatagagttatccttctaaagacttgttctttcagaaaaatgtttttccaattagtataaactctacaaattccctcttggtgtaaaattcaagatgtggaaattctaacttttttgaaatttaaaagcttatattttctaacttggcaaaagacttatccttagaaagagaagtgtacattgatttccaattaaaaatttgctggcattaaaaataagcacacttacataagcccccatacatagagtgggactcttggaatcaggagacaaagctaccacatgtggaaaggtactatgtgtccatgtcattcaaaaaatgtgattttttataataaactctttataacaagattaaaa

SEQ ID NO: 11 - DNA, 1499 bp, Artificial Sequence
  Human ARG1 transcript variant 1 mRNA sequence
ggaaaaaaaagatgcgccctctgtcactgagggttgactgactggagagctcaagtgcagcaaagagaagtgtcagagcatgagcgccaagtccagaaccatagggattattggagctcctttctcaaagggacagccacgaggaggggtggaagaaggccctacagtattgagaaaggctggtctgcttgagaaacttaaagaacaagtaactcaaaactttttaattttagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaaatgtggaaacatccgatataaatctcatagttaatggcataattagaaagctaatcattttcttaagcatagagttatccttctaaagacttgttctttcagaaaaatgtttttccaattagtataaactctacaaattccctcttggtgtaaaattcaagatgtggaaattctaacttttttgaaatttaaaagcttatattttctaacttggcaaaagacttatccttagaaagagaagtgtacattgatttccaattaaaaatttgctggcattaaaaataagcacacttacataagcccccatacatagagtgggactcttggaatcaggagacaaagctaccacatgtggaaaggtactatgtgtccatgtcattcaaaaaatgtgattttttataataaactctttataacaagattaaaa

SEQ ID NO: 12 - DNA, 8554 bp, Artificial Sequence
  Human CREB5 transcript variant 1 mRNA sequence
aacatttacaacaaagttgattctgtgtagggttggaggctagacagttccacaaatttttagtcacattttccatgtcagttaaatctagggagttcaagactactggaaaaattagtctcattactaaaagaaacttagagaacgagggaggtaccagagtctaggaggtacctctgggttgcagaagtaattgtaaaataccagacctgttctttttactaaaagctagtttcactatcttctggtctgaaatactgaggcaaatactcaagacttattttcttcctaatcttgctggtgaaacagaagttactagaaagaaaggaagaaaaaacttgatttggtgactgcaggaagcaacacgttgctgcttttattctacagataatgatttatgaggaatccaagatgaatttggagcaggagaggccgtttgtctgcagtgccccaggctgctcccagcgcttcccaacagaggaccatctgatgattcataggcacaaacatgaaatgactttgaagtttccttcaataaaaacagacaatatgttatcagatcaaactccgaccccaacgagattcctgaagaactgcgaggaggtgggcctcttcagcgagctggactgctccctggagcacgagttcaggaaggctcaggaagaggagagcagcaagcggaatatctcgatgcataatgcagttggtggggccatgacggggcccggaactcaccagcttagcagcgctcggctgcccaaccatgacaccaacgttgtgattcagcaagccatgccgtcgcctcagtccagctctgtcatcactcaggcaccttccaccaaccgccagatcgggcctgtcccaggctctctatcttctctgctacatctccacaacagacagagacagcccatgccagcctccatgcctgggaccctgcccaaccctacaatgccaggatcttccgccgtcttgatgccaatggagcgacaaatgtcagtgaactccagcatcatggggatgcaaggtccaaatctcagcaacccctgtgcttctccccaggtccagccaatgcattcagaagccaaaatgaggttgaaggctgcattgactcaccaccctgctgccatgtcaaatgggaacatgaacaccatgggacacatgatggagatgatgggctcccggcaggaccagacgccacaccatcacatgcactcgcacccgcatcagcaccagacactgccaccccatcacccttacccacaccagcaccagcacccagcacaccatcctcaccctcaaccccatcaccagcagaaccatccacatcaccactcccattcccaccttcatgcacacccagcacatcaccagacctcgccacatccgcccctgcacaccggcaaccaagcacaggtttcaccagcaacacaacagatgcagccaacccagacaatacagccaccccagcccacaggggggcgccggcgaagggtggtagacgaggatccggacgagaggcggcggaaatttctggaacggaaccgggcagctgccacccgctgcagacagaagaggaaggtctgggtgatgtcattggaaaagaaagcagaagaactcacccagacaaacatgcagcttcagaatgaagtgtctatgttgaaaaatgaggtggcccagctgaaacagttgttgttaacacataaagactgcccaataacagccatgcagaaagaatcacaaggatatctaagtccagagagtagccctcctgctagtcctgtcccagcttgctcccagcaacaagtcatccagcataataccatcactacttcctcatcggtcagcgaggtggtaggaagctccaccctcagccagctcaccactcacagaacagacctgaatccgattctttaaaatgcaccatcagacctggcctccaagaagagctgtagcgtaccatgcgtcctttcttttaagggcatttttagaattaactcagacctggaagactcctcagttcttcaaagactggctttcatttttatagttattatggaaatgttgtcttttatacttagttatataagaaaaaagggagttatgcaattaatatctatcagcttgggaaacgctttggtgcttttctccagttttctggtaccagttacttgtttataaactgaaccttttctgtatatagccatggtttcattcttatcagtccaaccctttgcctgaaacattgaatcttgttaaaccacagcttttagctaaaatgaggtatacctagatgtcaagtaagacagatccaaggtaactgggtaggaaatcttttgacatcttaactcatgttgagtttgtgctgtggtgtcaccagaattccagataaacacacagcctttcccatacctttttttttcttactataaaatattataagatccattgatgtccaaataataccaccaagcatctcttcacctctcctcctcttggtccacttgctaatgcccagttttcttctccatttccactttttcttaggctccctatttactattcattttgacttccttctgttttatttttttccctttagcattgcatgtgaataagaaaataatgtttaaagaaaaaaaaaaaaaagcaaacctccaaaacgtggacctaaccattgcttcacttacacttcacccacagctggagttcattcaactcttgcttttcacaaaatagtaaccaggagatgtttaatgtgcctgatttaatgtttttaataatcacagcaaatgaaaggtggtttagttataagtgaagcatggttgaataccagctggggagacactagggaagggagctttgtaagccttgattgcgaaagtccaaattttgatgtggggctataacatgacacccttggattgcgactggttttatacggcctgcctataacgttgaaaatccatgtactacataataattcagaagggctctattcactacacagattacattgttcaatcatcagctgctaatagcctaagatttatttttttttttttcttaagcctatggaaccggctttgctgttctggggggtgaaaatagactaactactggagaaacaaagagagaaagaaaacccagtgtttccataggggcacttttagccttcccacaacagttaagcactctttgactgctgaaggaaccccatggatgaggtgcaggctacttcactctttttttttcttttttgagacagagtctcacctattgcccagactgaagtgcagtggtgcgatcatggatcactgcagcagcatcctccgagttcaagctatccttccacctccgcctcctgagtagctgggaccacaggttcacataaccatgcctggctaatttatttttacttttattttaaaataaaagatgaggtctgtcttatgttgcccaggctggtctcaaactatcctacttcttcctcccaaagtgttgggattataggtgtgagccactgcacccagcctacttcactcttctgaattattctgatttattttcaacaacttttgtgaacttgcccgtgatacaaagcagatagtccctgaaccacagtcgtgcctccttgaaacaagccattctactgtgctaatgttttaatatcacatctcacaaataacaggggtgaatgtttctctctagcaatctaggcaggtgctggtgtttcatctccatttgaatgcttgacctcttaatgtgtgtgtgtgtgtgtgtgtgtgtgtgtgtgttcatgggttttaaaagaacagtattttacaaaaggtgtagcttttataagagtgcagaaaagggaaggatgtgtttttttctctcactatagtataagaatctattttggagaaaaaaagaaaatatgagggtctcgaagcatgatttttatataactagtttcagttttatctaataacttactttttaaatcaatatttatcaacaatctttccttgtatgcagtgctttcaaaagatggttttgagtgtccagtgaaacttatgacttggatatatggttgaagaatcaaaacaaaagcaaaaaaaaaaagcaaaaaaagaaaagagaaaaaaagaaaaaatgcaaatggaataattttctattatattttagacaaacatatcattttcgagtattttaaatactgaattcatagttgttgttttttaaattccaacagtaacagctgaatggtttaatctgactggcttcctaagaaatgtttaagactcagctttaaaaagaagttaacattcatatctctgttttgaaatcaaaaatcatatttcaaaattctttcctaggaccatctatgtgtctcccctcccctccacaaaaaggagaaagagtgcattaaaatgtttagttgggttttttaatttttaatttttatgttatgttttgctttgttttaagtaaacaaaaatttttctttctttactgcatgcatagcacttaataaaatggatttttaaaaaatccactagtaatatcagaatgtccagggagtgactgtcactacaatgatggtttagtttacttctgttccaccttttgattgaaatatttagttgttaggctgaaagcctcggcagttaagaacttgcctgagttttcttcgttcagcaacttgacagtttgactgatgtgcattatatatagctcaattatgtctgttttttatgctaagtaggaaaaccaaccacacacattagcaaaccggcctcaacatataattagaataaactgtcttcttgttctactcagggcctttaggtgtgttcattcacggtatggaaatacagtaaatgaaagattccaactagttgtcagtgcttcttgaaattccaaacagaaagatacattggtcaaatccaacacttggcttatcaatattaagtcttttacctaaaggcccagccgtcaccagacaacagaataatcaatctgcctgaaaatccctcctccttgtcctacactttttgcctgtttgggagaatatctttgtactccattctcctccctcagccagttactgggtcacccatccatgtgttcatgaatcaatcatcacggcctgcagagcacctgtcctaaggagggaaaatcctgtcacactgcctctccccattcgtgtgtggttttcttgatcggtgagatctgtctctgaagtcactgccagcctccctgggaacgtctatagtgcctcccctgccttatgtgatgggagttaacaactcagataagtacacctgagagcatttctatcaggtaaactgtcacttaaatggaggtgtccacatcttaattgtttctccttgacacatttctcaatccacgaagccaggagaggtagagtgaaaatcccagccatggatgaatgtactaatttgaaagccaagtgttaagtcggatgttttcccgttacactactactcagccctctcctgcggccacatcaacggatgcaagtcacagtcttaacacagcctgtgggagacaagcagtttgtgtgctcacagtatatattatagtaattagggtgacttagagcaaatactcttcagatcctatgtagtcagtgaaacaaaatggagagcgtattctgatagaaggacgtcgacggtgaatgttctggtggttgttgcctgttaagtaaactttagtgtgtaagttgagtttgtcattaaaatcataaaccagctgcggtaacagacaagcctttggctggggagttttaagcctcggtaactgctataaaactagccatccagttaggatagaatgtgtttctttctggttaaaaaaaggaaaaaccatctaagaaaatatatatgtatgtatgtgtgtatacagtggaattcaaaggaccaaagcaaaatttgaacaggaatctattaatttagaattttataagatatttattaataaatgttatttttaaacattccatttgaacagtattctgtaggatctacttgtttttaaagtgttagtccataataaactactatagttatgtgtattttcatttttcagggtttcaaatggctattctccatcatttggtggaaatgtttgcttagatctctgtgcatagacatttcaaggatttttattgctctgtgagttattttttaatcaacattctgaacagttttttttaaacatttatttctgtgtgttcatttttaaagtaagctctttcatttaggaagcagagttcagctaaagggaatcagtaactctaactggaacagctttcttgtagaagtgtaaaaacagcttcatctctgcctctctccaccccaccccaatttcctagaaagccttgcactattcagctcccttagtgctttttgtcccttcccgaacaatatgcagtagctttaagccattcaagctccattatgcagtatatctgagaagggaaaggaaacaacccatttaaatttgaataaaaccgtgcctatgcgaacagtagcaatttagaatctcttttctgcttttaaaataatttatatttaaaaattgcactttagctttttgatccctttgtatttctcttattctctttctaacctcttctctgtcctcaaacttgcctttgctctcctttacaataccccccacccctcctccaaggctctgagcggcatcatttaaaatactttacagatatttgcaccaggtacatttatgtgcgtccattggtagcacagctgagacctgtgtctcacatcagcctaggtgaagcctactacaagaatgccaaggagaagagccagtacactatatggtttatactctttatccctttattcatagcatgttttttaaaaatgttatattatgcaacagatgtgaggcagcagctaagctatacttaagaattttctctcaccttccaaaccaaagtgtcctgaataagccaggagacttattcttttgtgcaccctggtgcacatctgactgttgtcctagccatagactctctgaggccactgaaagaacagtggccctatcgatttcattcctaggtctcaaaaatacaatgttgccttgtaacataattagggacagcacctctatttcacaattataatctaaggtaggataagacgacacagcagcaataaacttacaagtaaaattcaataccaaaacaaacacaaagaaatttaaaaaacaaaaaacctagctcatcatgttgtgaaaatgaaaaagtgaatgtccattcaaaatattttactatttcttgtggagtttttcagtgatgtaatgcttgtagccaaattgcttaaagagtgtttatatatttttttccttataaattgtctattttttaaaaaagctatttaaccacagctgaagtggggggtaaggccaaattgccaacacttgttaaaagattaatactcttaagtggcactctgatacctttccaacttgtcatcagaaaggaatcaataattaccaactgttgtatttagaccaacttacaatatctagctcattagaagccaggatctagaaagctccttctaagccatttaagatattcttacattgagcttcatattatagaactttataggattggatattttacaatagaataatttagcctcaggactgagaatgtggaagctgaataaattagctttaaatacatcattaaaatcttatgcacaataagctcattagattctagttttctcctttagaataccaatgccacagacactacaggagataatgaaaggtatcagttgtgttgagtggagggagtttaagagaaaggacccttcccaaccagcagccagtagaaaatacaacctactcacctttttcccttctaagttctgctaaatcacatctgcctcatagagaaaggaatgttgcctttgagaactgtcttggagaacagataagcttgaaatgttctctctagagaggacatagggtttgggatcctctgaaaaggcccagaaaaatagctcagttcaaatacaatgttctaggacaattggaatataaatattgtccaaaaatataattaaaagaaaaaagtttagcactgtgtaaagtaagtgttaactgaggaagtcccaaaaaggtgctgtcactttaagttctggacttggggttctttgtatttgtaaacagcaaagcatttgtgtttgtttgtctatttgtaaagcaaccaccttccttattggaaggagaaaaaaaggggtacatacatgtaaatacttgctgcagcatttaatatgtttaattttgtgttaagctttttgttgcatcgtgaacacatttattgttaccaatggacaatgagttcattaagactgttcaactaggtcagatttttacatctctttctagcaagaagagacaagattttgtgcatttgtacaaatgttaatatcactgcaattccaatataataaagcactcaaatgcaaataaaaaaaaaaaaaaaaaaaaaaaa

SEQ ID NO: 13 - DNA, 7917 bp, Artificial Sequence
  Human CREB5 transcript variant 4 mRNA sequence
agagagcaagtgagcgagagcccagccggagggagagccagagctaggcgcaaaagaagggccagcacattacatcatcgcttggcctggaatcaaatgggaaggatatgactcactcaagctagttaagctttggctcaaattctacacacactcattccagagctctcatgttctgcacctcaggagggaattcagcctcagtgatgtccatgaggcctgtcccaggctctctatcttctctgctacatctccacaacagacagagacagcccatgccagcctccatgcctgggaccctgcccaaccctacaatgccaggatcttccgccgtcttgatgccaatggagcgacaaatgtcagtgaactccagcatcatggggatgcaaggtccaaatctcagcaacccctgtgcttctccccaggtccagccaatgcattcagaagccaaaatgaggttgaaggctgcattgactcaccaccctgctgccatgtcaaatgggaacatgaacaccatgggacacatgatggagatgatgggctcccggcaggaccagacgccacaccatcacatgcactcgcacccgcatcagcaccagacactgccaccccatcacccttacccacaccagcaccagcacccagcacaccatcctcaccctcaaccccatcaccagcagaaccatccacatcaccactcccattcccaccttcatgcacacccagcacatcaccagacctcgccacatccgcccctgcacaccggcaaccaagcacaggtttcaccagcaacacaacagatgcagccaacccagacaatacagccaccccagcccacaggggggcgccggcgaagggtggtagacgaggatccggacgagaggcggcggaaatttctggaacggaaccgggcagctgccacccgctgcagacagaagaggaaggtctgggtgatgtcattggaaaagaaagcagaagaactcacccagacaaacatgcagcttcagaatgaagtgtctatgttgaaaaatgaggtggcccagctgaaacagttgttgttaacacataaagactgcccaataacagccatgcagaaagaatcacaaggatatctaagtccagagagtagccctcctgctagtcctgtcccagcttgctcccagcaacaagtcatccagcataataccatcactacttcctcatcggtcagcgaggtggtaggaagctccaccctcagccagctcaccactcacagaacagacctgaatccgattctttaaaatgcaccatcagacctggcctccaagaagagctgtagcgtaccatgcgtcctttcttttaagggcatttttagaattaactcagacctggaagactcctcagttcttcaaagactggctttcatttttatagttattatggaaatgttgtcttttatacttagttatataagaaaaaagggagttatgcaattaatatctatcagcttgggaaacgctttggtgcttttctccagttttctggtaccagttacttgtttataaactgaaccttttctgtatatagccatggtttcattcttatcagtccaaccctttgcctgaaacattgaatcttgttaaaccacagcttttagctaaaatgaggtatacctagatgtcaagtaagacagatccaaggtaactgggtaggaaatcttttgacatcttaactcatgttgagtttgtgctgtggtgtcaccagaattccagataaacacacagcctttcccatacctttttttttcttactataaaatattataagatccattgatgtccaaataataccaccaagcatctcttcacctctcctcctcttggtccacttgctaatgcccagttttcttctccatttccactttttcttaggctccctatttactattcattttgacttccttctgttttatttttttccctttagcattgcatgtgaataagaaaataatgtttaaagaaaaaaaaaaaaaagcaaacctccaaaacgtggacctaaccattgcttcacttacacttcacccacagctggagttcattcaactcttgcttttcacaaaatagtaaccaggagatgtttaatgtgcctgatttaatgtttttaataatcacagcaaatgaaaggtggtttagttataagtgaagcatggttgaataccagctggggagacactagggaagggagctttgtaagccttgattgcgaaagtccaaattttgatgtggggctataacatgacacccttggattgcgactggttttatacggcctgcctataacgttgaaaatccatgtactacataataattcagaagggctctattcactacacagattacattgttcaatcatcagctgctaatagcctaagatttatttttttttttttcttaagcctatggaaccggctttgctgttctggggggtgaaaatagactaactactggagaaacaaagagagaaagaaaacccagtgtttccataggggcacttttagccttcccacaacagttaagcactctttgactgctgaaggaaccccatggatgaggtgcaggctacttcactctttttttttcttttttgagacagagtctcacctattgcccagactgaagtgcagtggtgcgatcatggatcactgcagcagcatcctccgagttcaagctatccttccacctccgcctcctgagtagctgggaccacaggttcacataaccatgcctggctaatttatttttacttttattttaaaataaaagatgaggtctgtcttatgttgcccaggctggtctcaaactatcctacttcttcctcccaaagtgttgggattataggtgtgagccactgcacccagcctacttcactcttctgaattattctgatttattttcaacaacttttgtgaacttgcccgtgatacaaagcagatagtccctgaaccacagtcgtgcctccttgaaacaagccattctactgtgctaatgttttaatatcacatctcacaaataacaggggtgaatgtttctctctagcaatctaggcaggtgctggtgtttcatctccatttgaatgcttgacctcttaatgtgtgtgtgtgtgtgtgtgtgtgtgtgtgtgttcatgggttttaaaagaacagtattttacaaaaggtgtagcttttataagagtgcagaaaagggaaggatgtgtttttttctctcactatagtataagaatctattttggagaaaaaaagaaaatatgagggtctcgaagcatgatttttatataactagtttcagttttatctaataacttactttttaaatcaatatttatcaacaatctttccttgtatgcagtgctttcaaaagatggttttgagtgtccagtgaaacttatgacttggatatatggttgaagaatcaaaacaaaagcaaaaaaaaaaagcaaaaaaagaaaagagaaaaaaagaaaaaatgcaaatggaataattttctattatattttagacaaacatatcattttcgagtattttaaatactgaattcatagttgttgttttttaaattccaacagtaacagctgaatggtttaatctgactggcttcctaagaaatgtttaagactcagctttaaaaagaagttaacattcatatctctgttttgaaatcaaaaatcatatttcaaaattctttcctaggaccatctatgtgtctcccctcccctccacaaaaaggagaaagagtgcattaaaatgtttagttgggttttttaatttttaatttttatgttatgttttgctttgttttaagtaaacaaaaatttttctttctttactgcatgcatagcacttaataaaatggatttttaaaaaatccactagtaatatcagaatgtccagggagtgactgtcactacaatgatggtttagtttacttctgttccaccttttgattgaaatatttagttgttaggctgaaagcctcggcagttaagaacttgcctgagttttcttcgttcagcaacttgacagtttgactgatgtgcattatatatagctcaattatgtctgttttttatgctaagtaggaaaaccaaccacacacattagcaaaccggcctcaacatataattagaataaactgtcttcttgttctactcagggcctttaggtgtgttcattcacggtatggaaatacagtaaatgaaagattccaactagttgtcagtgcttcttgaaattccaaacagaaagatacattggtcaaatccaacacttggcttatcaatattaagtcttttacctaaaggcccagccgtcaccagacaacagaataatcaatctgcctgaaaatccctcctccttgtcctacactttttgcctgtttgggagaatatctttgtactccattctcctccctcagccagttactgggtcacccatccatgtgttcatgaatcaatcatcacggcctgcagagcacctgtcctaaggagggaaaatcctgtcacactgcctctccccattcgtgtgtggttttcttgatcggtgagatctgtctctgaagtcactgccagcctccctgggaacgtctatagtgcctcccctgccttatgtgatgggagttaacaactcagataagtacacctgagagcatttctatcaggtaaactgtcacttaaatggaggtgtccacatcttaattgtttctccttgacacatttctcaatccacgaagccaggagaggtagagtgaaaatcccagccatggatgaatgtactaatttgaaagccaagtgttaagtcggatgttttcccgttacactactactcagccctctcctgcggccacatcaacggatgcaagtcacagtcttaacacagcctgtgggagacaagcagtttgtgtgctcacagtatatattatagtaattagggtgacttagagcaaatactcttcagatcctatgtagtcagtgaaacaaaatggagagcgtattctgatagaaggacgtcgacggtgaatgttctggtggttgttgcctgttaagtaaactttagtgtgtaagttgagtttgtcattaaaatcataaaccagctgcggtaacagacaagcctttggctggggagttttaagcctcggtaactgctataaaactagccatccagttaggatagaatgtgtttctttctggttaaaaaaaggaaaaaccatctaagaaaatatatatgtatgtatgtgtgtatacagtggaattcaaaggaccaaagcaaaatttgaacaggaatctattaatttagaattttataagatatttattaataaatgttatttttaaacattccatttgaacagtattctgtaggatctacttgtttttaaagtgttagtccataataaactactatagttatgtgtattttcatttttcagggtttcaaatggctattctccatcatttggtggaaatgtttgcttagatctctgtgcatagacatttcaaggatttttattgctctgtgagttattttttaatcaacattctgaacagttttttttaaacatttatttctgtgtgttcatttttaaagtaagctctttcatttaggaagcagagttcagctaaagggaatcagtaactctaactggaacagctttcttgtagaagtgtaaaaacagcttcatctctgcctctctccaccccaccccaatttcctagaaagccttgcactattcagctcccttagtgctttttgtcccttcccgaacaatatgcagtagctttaagccattcaagctccattatgcagtatatctgagaagggaaaggaaacaacccatttaaatttgaataaaaccgtgcctatgcgaacagtagcaatttagaatctcttttctgcttttaaaataatttatatttaaaaattgcactttagctttttgatccctttgtatttctcttattctctttctaacctcttctctgtcctcaaacttgcctttgctctcctttacaataccccccacccctcctccaaggctctgagcggcatcatttaaaatactttacagatatttgcaccaggtacatttatgtgcgtccattggtagcacagctgagacctgtgtctcacatcagcctaggtgaagcctactacaagaatgccaaggagaagagccagtacactatatggtttatactctttatccctttattcatagcatgttttttaaaaatgttatattatgcaacagatgtgaggcagcagctaagctatacttaagaattttctctcaccttccaaaccaaagtgtcctgaataagccaggagacttattcttttgtgcaccctggtgcacatctgactgttgtcctagccatagactctctgaggccactgaaagaacagtggccctatcgatttcattcctaggtctcaaaaatacaatgttgccttgtaacataattagggacagcacctctatttcacaattataatctaaggtaggataagacgacacagcagcaataaacttacaagtaaaattcaataccaaaacaaacacaaagaaatttaaaaaacaaaaaacctagctcatcatgttgtgaaaatgaaaaagtgaatgtccattcaaaatattttactatttcttgtggagtttttcagtgatgtaatgcttgtagccaaattgcttaaagagtgtttatatatttttttccttataaattgtctattttttaaaaaagctatttaaccacagctgaagtggggggtaaggccaaattgccaacacttgttaaaagattaatactcttaagtggcactctgatacctttccaacttgtcatcagaaaggaatcaataattaccaactgttgtatttagaccaacttacaatatctagctcattagaagccaggatctagaaagctccttctaagccatttaagatattcttacattgagcttcatattatagaactttataggattggatattttacaatagaataatttagcctcaggactgagaatgtggaagctgaataaattagctttaaatacatcattaaaatcttatgcacaataagctcattagattctagttttctcctttagaataccaatgccacagacactacaggagataatgaaaggtatcagttgtgttgagtggagggagtttaagagaaaggacccttcccaaccagcagccagtagaaaatacaacctactcacctttttcccttctaagttctgctaaatcacatctgcctcatagagaaaggaatgttgcctttgagaactgtcttggagaacagataagcttgaaatgttctctctagagaggacatagggtttgggatcctctgaaaaggcccagaaaaatagctcagttcaaatacaatgttctaggacaattggaatataaatattgtccaaaaatataattaaaagaaaaaagtttagcactgtgtaaagtaagtgttaactgaggaagtcccaaaaaggtgctgtcactttaagttctggacttggggttctttgtatttgtaaacagcaaagcatttgtgtttgtttgtctatttgtaaagcaaccaccttccttattggaaggagaaaaaaaggggtacatacatgtaaatacttgctgcagcatttaatatgtttaattttgtgttaagctttttgttgcatcgtgaacacatttattgttaccaatggacaatgagttcattaagactgttcaactaggtcagatttttacatctctttctagcaagaagagacaagattttgtgcatttgtacaaatgttaatatcactgcaattccaatataataaagcactcaaatgcaaataaaaaaaaaaaaaaaaaaaaaaaa

SEQ ID NO: 14 - DNA, 8210 bp, Artificial Sequence
  Human CREB5 transcript variant 3 mRNA sequence
gatctgatgaatccaaggagtggagcaagaggcagattttggacacggttatgagaatgacagaaactgcctaaagcatttatgctctggcattcgtccctgtttctggaggtccagtaagcgcttcccaacagaggaccatctgatgattcataggcacaaacatgaaatgactttgaagtttccttcaataaaaacagacaatatgttatcagatcaaactccgaccccaacgagattcctgaagaactgcgaggaggtgggcctcttcagcgagctggactgctccctggagcacgagttcaggaaggctcaggaagaggagagcagcaagcggaatatctcgatgcataatgcagttggtggggccatgacggggcccggaactcaccagcttagcagcgctcggctgcccaaccatgacaccaacgttgtgattcagcaagccatgccgtcgcctcagtccagctctgtcatcactcaggcaccttccaccaaccgccagatcgggcctgtcccaggctctctatcttctctgctacatctccacaacagacagagacagcccatgccagcctccatgcctgggaccctgcccaaccctacaatgccaggatcttccgccgtcttgatgccaatggagcgacaaatgtcagtgaactccagcatcatggggatgcaaggtccaaatctcagcaacccctgtgcttctccccaggtccagccaatgcattcagaagccaaaatgaggttgaaggctgcattgactcaccaccctgctgccatgtcaaatgggaacatgaacaccatgggacacatgatggagatgatgggctcccggcaggaccagacgccacaccatcacatgcactcgcacccgcatcagcaccagacactgccaccccatcacccttacccacaccagcaccagcacccagcacaccatcctcaccctcaaccccatcaccagcagaaccatccacatcaccactcccattcccaccttcatgcacacccagcacatcaccagacctcgccacatccgcccctgcacaccggcaaccaagcacaggtttcaccagcaacacaacagatgcagccaacccagacaatacagccaccccagcccacaggggggcgccggcgaagggtggtagacgaggatccggacgagaggcggcggaaatttctggaacggaaccgggcagctgccacccgctgcagacagaagaggaaggtctgggtgatgtcattggaaaagaaagcagaagaactcacccagacaaacatgcagcttcagaatgaagtgtctatgttgaaaaatgaggtggcccagctgaaacagttgttgttaacacataaagactgcccaataacagccatgcagaaagaatcacaaggatatctaagtccagagagtagccctcctgctagtcctgtcccagcttgctcccagcaacaagtcatccagcataataccatcactacttcctcatcggtcagcgaggtggtaggaagctccaccctcagccagctcaccactcacagaacagacctgaatccgattctttaaaatgcaccatcagacctggcctccaagaagagctgtagcgtaccatgcgtcctttcttttaagggcatttttagaattaactcagacctggaagactcctcagttcttcaaagactggctttcatttttatagttattatggaaatgttgtcttttatacttagttatataagaaaaaagggagttatgcaattaatatctatcagcttgggaaacgctttggtgcttttctccagttttctggtaccagttacttgtttataaactgaaccttttctgtatatagccatggtttcattcttatcagtccaaccctttgcctgaaacattgaatcttgttaaaccacagcttttagctaaaatgaggtatacctagatgtcaagtaagacagatccaaggtaactgggtaggaaatcttttgacatcttaactcatgttgagtttgtgctgtggtgtcaccagaattccagataaacacacagcctttcccatacctttttttttcttactataaaatattataagatccattgatgtccaaataataccaccaagcatctcttcacctctcctcctcttggtccacttgctaatgcccagttttcttctccatttccactttttcttaggctccctatttactattcattttgacttccttctgttttatttttttccctttagcattgcatgtgaataagaaaataatgtttaaagaaaaaaaaaaaaaagcaaacctccaaaacgtggacctaaccattgcttcacttacacttcacccacagctggagttcattcaactcttgcttttcacaaaatagtaaccaggagatgtttaatgtgcctgatttaatgtttttaataatcacagcaaatgaaaggtggtttagttataagtgaagcatggttgaataccagctggggagacactagggaagggagctttgtaagccttgattgcgaaagtccaaattttgatgtggggctataacatgacacccttggattgcgactggttttatacggcctgcctataacgttgaaaatccatgtactacataataattcagaagggctctattcactacacagattacattgttcaatcatcagctgctaatagcctaagatttatttttttttttttcttaagcctatggaaccggctttgctgttctggggggtgaaaatagactaactactggagaaacaaagagagaaagaaaacccagtgtttccataggggcacttttagccttcccacaacagttaagcactctttgactgctgaaggaaccccatggatgaggtgcaggctacttcactctttttttttcttttttgagacagagtctcacctattgcccagactgaagtgcagtggtgcgatcatggatcactgcagcagcatcctccgagttcaagctatccttccacctccgcctcctgagtagctgggaccacaggttcacataaccatgcctggctaatttatttttacttttattttaaaataaaagatgaggtctgtcttatgttgcccaggctggtctcaaactatcctacttcttcctcccaaagtgttgggattataggtgtgagccactgcacccagcctacttcactcttctgaattattctgatttattttcaacaacttttgtgaacttgcccgtgatacaaagcagatagtccctgaaccacagtcgtgcctccttgaaacaagccattctactgtgctaatgttttaatatcacatctcacaaataacaggggtgaatgtttctctctagcaatctaggcaggtgctggtgtttcatctccatttgaatgcttgacctcttaatgtgtgtgtgtgtgtgtgtgtgtgtgtgtgtgttcatgggttttaaaagaacagtattttacaaaaggtgtagcttttataagagtgcagaaaagggaaggatgtgtttttttctctcactatagtataagaatctattttggagaaaaaaagaaaatatgagggtctcgaagcatgatttttatataactagtttcagttttatctaataacttactttttaaatcaatatttatcaacaatctttccttgtatgcagtgctttcaaaagatggttttgagtgtccagtgaaacttatgacttggatatatggttgaagaatcaaaacaaaagcaaaaaaaaaaagcaaaaaaagaaaagagaaaaaaagaaaaaatgcaaatggaataattttctattatattttagacaaacatatcattttcgagtattttaaatactgaattcatagttgttgttttttaaattccaacagtaacagctgaatggtttaatctgactggcttcctaagaaatgtttaagactcagctttaaaaagaagttaacattcatatctctgttttgaaatcaaaaatcatatttcaaaattctttcctaggaccatctatgtgtctcccctcccctccacaaaaaggagaaagagtgcattaaaatgtttagttgggttttttaatttttaatttttatgttatgttttgctttgttttaagtaaacaaaaatttttctttctttactgcatgcatagcacttaataaaatggatttttaaaaaatccactagtaatatcagaatgtccagggagtgactgtcactacaatgatggtttagtttacttctgttccaccttttgattgaaatatttagttgttaggctgaaagcctcggcagttaagaacttgcctgagttttcttcgttcagcaacttgacagtttgactgatgtgcattatatatagctcaattatgtctgttttttatgctaagtaggaaaaccaaccacacacattagcaaaccggcctcaacatataattagaataaactgtcttcttgttctactcagggcctttaggtgtgttcattcacggtatggaaatacagtaaatgaaagattccaactagttgtcagtgcttcttgaaattccaaacagaaagatacattggtcaaatccaacacttggcttatcaatattaagtcttttacctaaaggcccagccgtcaccagacaacagaataatcaatctgcctgaaaatccctcctccttgtcctacactttttgcctgtttgggagaatatctttgtactccattctcctccctcagccagttactgggtcacccatccatgtgttcatgaatcaatcatcacggcctgcagagcacctgtcctaaggagggaaaatcctgtcacactgcctctccccattcgtgtgtggttttcttgatcggtgagatctgtctctgaagtcactgccagcctccctgggaacgtctatagtgcctcccctgccttatgtgatgggagttaacaactcagataagtacacctgagagcatttctatcaggtaaactgtcacttaaatggaggtgtccacatcttaattgtttctccttgacacatttctcaatccacgaagccaggagaggtagagtgaaaatcccagccatggatgaatgtactaatttgaaagccaagtgttaagtcggatgttttcccgttacactactactcagccctctcctgcggccacatcaacggatgcaagtcacagtcttaacacagcctgtgggagacaagcagtttgtgtgctcacagtatatattatagtaattagggtgacttagagcaaatactcttcagatcctatgtagtcagtgaaacaaaatggagagcgtattctgatagaaggacgtcgacggtgaatgttctggtggttgttgcctgttaagtaaactttagtgtgtaagttgagtttgtcattaaaatcataaaccagctgcggtaacagacaagcctttggctggggagttttaagcctcggtaactgctataaaactagccatccagttaggatagaatgtgtttctttctggttaaaaaaaggaaaaaccatctaagaaaatatatatgtatgtatgtgtgtatacagtggaattcaaaggaccaaagcaaaatttgaacaggaatctattaatttagaattttataagatatttattaataaatgttatttttaaacattccatttgaacagtattctgtaggatctacttgtttttaaagtgttagtccataataaactactatagttatgtgtattttcatttttcagggtttcaaatggctattctccatcatttggtggaaatgtttgcttagatctctgtgcatagacatttcaaggatttttattgctctgtgagttattttttaatcaacattctgaacagttttttttaaacatttatttctgtgtgttcatttttaaagtaagctctttcatttaggaagcagagttcagctaaagggaatcagtaactctaactggaacagctttcttgtagaagtgtaaaaacagcttcatctctgcctctctccaccccaccccaatttcctagaaagccttgcactattcagctcccttagtgctttttgtcccttcccgaacaatatgcagtagctttaagccattcaagctccattatgcagtatatctgagaagggaaaggaaacaacccatttaaatttgaataaaaccgtgcctatgcgaacagtagcaatttagaatctcttttctgcttttaaaataatttatatttaaaaattgcactttagctttttgatccctttgtatttctcttattctctttctaacctcttctctgtcctcaaacttgcctttgctctcctttacaataccccccacccctcctccaaggctctgagcggcatcatttaaaatactttacagatatttgcaccaggtacatttatgtgcgtccattggtagcacagctgagacctgtgtctcacatcagcctaggtgaagcctactacaagaatgccaaggagaagagccagtacactatatggtttatactctttatccctttattcatagcatgttttttaaaaatgttatattatgcaacagatgtgaggcagcagctaagctatacttaagaattttctctcaccttccaaaccaaagtgtcctgaataagccaggagacttattcttttgtgcaccctggtgcacatctgactgttgtcctagccatagactctctgaggccactgaaagaacagtggccctatcgatttcattcctaggtctcaaaaatacaatgttgccttgtaacataattagggacagcacctctatttcacaattataatctaaggtaggataagacgacacagcagcaataaacttacaagtaaaattcaataccaaaacaaacacaaagaaatttaaaaaacaaaaaacctagctcatcatgttgtgaaaatgaaaaagtgaatgtccattcaaaatattttactatttcttgtggagtttttcagtgatgtaatgcttgtagccaaattgcttaaagagtgtttatatatttttttccttataaattgtctattttttaaaaaagctatttaaccacagctgaagtggggggtaaggccaaattgccaacacttgttaaaagattaatactcttaagtggcactctgatacctttccaacttgtcatcagaaaggaatcaataattaccaactgttgtatttagaccaacttacaatatctagctcattagaagccaggatctagaaagctccttctaagccatttaagatattcttacattgagcttcatattatagaactttataggattggatattttacaatagaataatttagcctcaggactgagaatgtggaagctgaataaattagctttaaatacatcattaaaatcttatgcacaataagctcattagattctagttttctcctttagaataccaatgccacagacactacaggagataatgaaaggtatcagttgtgttgagtggagggagtttaagagaaaggacccttcccaaccagcagccagtagaaaatacaacctactcacctttttcccttctaagttctgctaaatcacatctgcctcatagagaaaggaatgttgcctttgagaactgtcttggagaacagataagcttgaaatgttctctctagagaggacatagggtttgggatcctctgaaaaggcccagaaaaatagctcagttcaaatacaatgttctaggacaattggaatataaatattgtccaaaaatataattaaaagaaaaaagtttagcactgtgtaaagtaagtgttaactgaggaagtcccaaaaaggtgctgtcactttaagttctggacttggggttctttgtatttgtaaacagcaaagcatttgtgtttgtttgtctatttgtaaagcaaccaccttccttattggaaggagaaaaaaaggggtacatacatgtaaatacttgctgcagcatttaatatgtttaattttgtgttaagctttttgttgcatcgtgaacacatttattgttaccaatggacaatgagttcattaagactgttcaactaggtcagatttttacatctctttctagcaagaagagacaagattttgtgcatttgtacaaatgttaatatcactgcaattccaatataataaagcactcaaatgcaaataaaaaaaaaaaaaaaaaaaaaaaa

SEQ ID NO: 15 - DNA, 8235 bp, Artificial Sequence
  Human CREB5 transcript variant 2 mRNA sequence
ttttagtggtggagtcaatttatttctgaaacgatctcatttacctgaatgaggagctcatatttattttcaggatttatgaggaatccaagatgaatttggagcaggagaggccgtttgtctgcagtgccccaggctgctcccagcgcttcccaacagaggaccatctgatgattcataggcacaaacatgaaatgactttgaagtttccttcaataaaaacagacaatatgttatcagatcaaactccgaccccaacgagattcctgaagaactgcgaggaggtgggcctcttcagcgagctggactgctccctggagcacgagttcaggaaggctcaggaagaggagagcagcaagcggaatatctcgatgcataatgcagttggtggggccatgacggggcccggaactcaccagcttagcagcgctcggctgcccaaccatgacaccaacgttgtgattcagcaagccatgccgtcgcctcagtccagctctgtcatcactcaggcaccttccaccaaccgccagatcgggcctgtcccaggctctctatcttctctgctacatctccacaacagacagagacagcccatgccagcctccatgcctgggaccctgcccaaccctacaatgccaggatcttccgccgtcttgatgccaatggagcgacaaatgtcagtgaactccagcatcatggggatgcaaggtccaaatctcagcaacccctgtgcttctccccaggtccagccaatgcattcagaagccaaaatgaggttgaaggctgcattgactcaccaccctgctgccatgtcaaatgggaacatgaacaccatgggacacatgatggagatgatgggctcccggcaggaccagacgccacaccatcacatgcactcgcacccgcatcagcaccagacactgccaccccatcacccttacccacaccagcaccagcacccagcacaccatcctcaccctcaaccccatcaccagcagaaccatccacatcaccactcccattcccaccttcatgcacacccagcacatcaccagacctcgccacatccgcccctgcacaccggcaaccaagcacaggtttcaccagcaacacaacagatgcagccaacccagacaatacagccaccccagcccacaggggggcgccggcgaagggtggtagacgaggatccggacgagaggcggcggaaatttctggaacggaaccgggcagctgccacccgctgcagacagaagaggaaggtctgggtgatgtcattggaaaagaaagcagaagaactcacccagacaaacatgcagcttcagaatgaagtgtctatgttgaaaaatgaggtggcccagctgaaacagttgttgttaacacataaagactgcccaataacagccatgcagaaagaatcacaaggatatctaagtccagagagtagccctcctgctagtcctgtcccagcttgctcccagcaacaagtcatccagcataataccatcactacttcctcatcggtcagcgaggtggtaggaagctccaccctcagccagctcaccactcacagaacagacctgaatccgattctttaaaatgcaccatcagacctggcctccaagaagagctgtagcgtaccatgcgtcctttcttttaagggcatttttagaattaactcagacctggaagactcctcagttcttcaaagactggctttcatttttatagttattatggaaatgttgtcttttatacttagttatataagaaaaaagggagttatgcaattaatatctatcagcttgggaaacgctttggtgcttttctccagttttctggtaccagttacttgtttataaactgaaccttttctgtatatagccatggtttcattcttatcagtccaaccctttgcctgaaacattgaatcttgttaaaccacagcttttagctaaaatgaggtatacctagatgtcaagtaagacagatccaaggtaactgggtaggaaatcttttgacatcttaactcatgttgagtttgtgctgtggtgtcaccagaattccagataaacacacagcctttcccatacctttttttttcttactataaaatattataagatccattgatgtccaaataataccaccaagcatctcttcacctctcctcctcttggtccacttgctaatgcccagttttcttctccatttccactttttcttaggctccctatttactattcattttgacttccttctgttttatttttttccctttagcattgcatgtgaataagaaaataatgtttaaagaaaaaaaaaaaaaagcaaacctccaaaacgtggacctaaccattgcttcacttacacttcacccacagctggagttcattcaactcttgcttttcacaaaatagtaaccaggagatgtttaatgtgcctgatttaatgtttttaataatcacagcaaatgaaaggtggtttagttataagtgaagcatggttgaataccagctggggagacactagggaagggagctttgtaagccttgattgcgaaagtccaaattttgatgtggggctataacatgacacccttggattgcgactggttttatacggcctgcctataacgttgaaaatccatgtactacataataattcagaagggctctattcactacacagattacattgttcaatcatcagctgctaatagcctaagatttatttttttttttttcttaagcctatggaaccggctttgctgttctggggggtgaaaatagactaactactggagaaacaaagagagaaagaaaacccagtgtttccataggggcacttttagccttcccacaacagttaagcactctttgactgctgaaggaaccccatggatgaggtgcaggctacttcactctttttttttcttttttgagacagagtctcacctattgcccagactgaagtgcagtggtgcgatcatggatcactgcagcagcatcctccgagttcaagctatccttccacctccgcctcctgagtagctgggaccacaggttcacataaccatgcctggctaatttatttttacttttattttaaaataaaagatgaggtctgtcttatgttgcccaggctggtctcaaactatcctacttcttcctcccaaagtgttgggattataggtgtgagccactgcacccagcctacttcactcttctgaattattctgatttattttcaacaacttttgtgaacttgcccgtgatacaaagcagatagtccctgaaccacagtcgtgcctccttgaaacaagccattctactgtgctaatgttttaatatcacatctcacaaataacaggggtgaatgtttctctctagcaatctaggcaggtgctggtgtttcatctccatttgaatgcttgacctcttaatgtgtgtgtgtgtgtgtgtgtgtgtgtgtgtgttcatgggttttaaaagaacagtattttacaaaaggtgtagcttttataagagtgcagaaaagggaaggatgtgtttttttctctcactatagtataagaatctattttggagaaaaaaagaaaatatgagggtctcgaagcatgatttttatataactagtttcagttttatctaataacttactttttaaatcaatatttatcaacaatctttccttgtatgcagtgctttcaaaagatggttttgagtgtccagtgaaacttatgacttggatatatggttgaagaatcaaaacaaaagcaaaaaaaaaaagcaaaaaaagaaaagagaaaaaaagaaaaaatgcaaatggaataattttctattatattttagacaaacatatcattttcgagtattttaaatactgaattcatagttgttgttttttaaattccaacagtaacagctgaatggtttaatctgactggcttcctaagaaatgtttaagactcagctttaaaaagaagttaacattcatatctctgttttgaaatcaaaaatcatatttcaaaattctttcctaggaccatctatgtgtctcccctcccctccacaaaaaggagaaagagtgcattaaaatgtttagttgggttttttaatttttaatttttatgttatgttttgctttgttttaagtaaacaaaaatttttctttctttactgcatgcatagcacttaataaaatggatttttaaaaaatccactagtaatatcagaatgtccagggagtgactgtcactacaatgatggtttagtttacttctgttccaccttttgattgaaatatttagttgttaggctgaaagcctcggcagttaagaacttgcctgagttttcttcgttcagcaacttgacagtttgactgatgtgcattatatatagctcaattatgtctgttttttatgctaagtaggaaaaccaaccacacacattagcaaaccggcctcaacatataattagaataaactgtcttcttgttctactcagggcctttaggtgtgttcattcacggtatggaaatacagtaaatgaaagattccaactagttgtcagtgcttcttgaaattccaaacagaaagatacattggtcaaatccaacacttggcttatcaatattaagtcttttacctaaaggcccagccgtcaccagacaacagaataatcaatctgcctgaaaatccctcctccttgtcctacactttttgcctgtttgggagaatatctttgtactccattctcctccctcagccagttactgggtcacccatccatgtgttcatgaatcaatcatcacggcctgcagagcacctgtcctaaggagggaaaatcctgtcacactgcctctccccattcgtgtgtggttttcttgatcggtgagatctgtctctgaagtcactgccagcctccctgggaacgtctatagtgcctcccctgccttatgtgatgggagttaacaactcagataagtacacctgagagcatttctatcaggtaaactgtcacttaaatggaggtgtccacatcttaattgtttctccttgacacatttctcaatccacgaagccaggagaggtagagtgaaaatcccagccatggatgaatgtactaatttgaaagccaagtgttaagtcggatgttttcccgttacactactactcagccctctcctgcggccacatcaacggatgcaagtcacagtcttaacacagcctgtgggagacaagcagtttgtgtgctcacagtatatattatagtaattagggtgacttagagcaaatactcttcagatcctatgtagtcagtgaaacaaaatggagagcgtattctgatagaaggacgtcgacggtgaatgttctggtggttgttgcctgttaagtaaactttagtgtgtaagttgagtttgtcattaaaatcataaaccagctgcggtaacagacaagcctttggctggggagttttaagcctcggtaactgctataaaactagccatccagttaggatagaatgtgtttctttctggttaaaaaaaggaaaaaccatctaagaaaatatatatgtatgtatgtgtgtatacagtggaattcaaaggaccaaagcaaaatttgaacaggaatctattaatttagaattttataagatatttattaataaatgttatttttaaacattccatttgaacagtattctgtaggatctacttgtttttaaagtgttagtccataataaactactatagttatgtgtattttcatttttcagggtttcaaatggctattctccatcatttggtggaaatgtttgcttagatctctgtgcatagacatttcaaggatttttattgctctgtgagttattttttaatcaacattctgaacagttttttttaaacatttatttctgtgtgttcatttttaaagtaagctctttcatttaggaagcagagttcagctaaagggaatcagtaactctaactggaacagctttcttgtagaagtgtaaaaacagcttcatctctgcctctctccaccccaccccaatttcctagaaagccttgcactattcagctcccttagtgctttttgtcccttcccgaacaatatgcagtagctttaagccattcaagctccattatgcagtatatctgagaagggaaaggaaacaacccatttaaatttgaataaaaccgtgcctatgcgaacagtagcaatttagaatctcttttctgcttttaaaataatttatatttaaaaattgcactttagctttttgatccctttgtatttctcttattctctttctaacctcttctctgtcctcaaacttgcctttgctctcctttacaataccccccacccctcctccaaggctctgagcggcatcatttaaaatactttacagatatttgcaccaggtacatttatgtgcgtccattggtagcacagctgagacctgtgtctcacatcagcctaggtgaagcctactacaagaatgccaaggagaagagccagtacactatatggtttatactctttatccctttattcatagcatgttttttaaaaatgttatattatgcaacagatgtgaggcagcagctaagctatacttaagaattttctctcaccttccaaaccaaagtgtcctgaataagccaggagacttattcttttgtgcaccctggtgcacatctgactgttgtcctagccatagactctctgaggccactgaaagaacagtggccctatcgatttcattcctaggtctcaaaaatacaatgttgccttgtaacataattagggacagcacctctatttcacaattataatctaaggtaggataagacgacacagcagcaataaacttacaagtaaaattcaataccaaaacaaacacaaagaaatttaaaaaacaaaaaacctagctcatcatgttgtgaaaatgaaaaagtgaatgtccattcaaaatattttactatttcttgtggagtttttcagtgatgtaatgcttgtagccaaattgcttaaagagtgtttatatatttttttccttataaattgtctattttttaaaaaagctatttaaccacagctgaagtggggggtaaggccaaattgccaacacttgttaaaagattaatactcttaagtggcactctgatacctttccaacttgtcatcagaaaggaatcaataattaccaactgttgtatttagaccaacttacaatatctagctcattagaagccaggatctagaaagctccttctaagccatttaagatattcttacattgagcttcatattatagaactttataggattggatattttacaatagaataatttagcctcaggactgagaatgtggaagctgaataaattagctttaaatacatcattaaaatcttatgcacaataagctcattagattctagttttctcctttagaataccaatgccacagacactacaggagataatgaaaggtatcagttgtgttgagtggagggagtttaagagaaaggacccttcccaaccagcagccagtagaaaatacaacctactcacctttttcccttctaagttctgctaaatcacatctgcctcatagagaaaggaatgttgcctttgagaactgtcttggagaacagataagcttgaaatgttctctctagagaggacatagggtttgggatcctctgaaaaggcccagaaaaatagctcagttcaaatacaatgttctaggacaattggaatataaatattgtccaaaaatataattaaaagaaaaaagtttagcactgtgtaaagtaagtgttaactgaggaagtcccaaaaaggtgctgtcactttaagttctggacttggggttctttgtatttgtaaacagcaaagcatttgtgtttgtttgtctatttgtaaagcaaccaccttccttattggaaggagaaaaaaaggggtacatacatgtaaatacttgctgcagcatttaatatgtttaattttgtgttaagctttttgttgcatcgtgaacacatttattgttaccaatggacaatgagttcattaagactgttcaactaggtcagatttttacatctctttctagcaagaagagacaagattttgtgcatttgtacaaatgttaatatcactgcaattccaatataataaagcactcaaatgcaaataaaaaaaaaaaaaaaaaaaaaaaa

SEQ ID NO: 16 - DNA, 602 bp, Artificial Sequence
  Human VPREB3 mRNA sequence
cttcccagccctgtgccccaaagcacctggagcatatagccttgcagaacttctacttgcctgcctccctgcctctggccatggcctgccggtgcctcagcttccttctgatggggaccttcctgtcagtttcccagacagtcctggcccagctggatgcactgctggtcttcccaggccaagtggctcaactctcctgcacgctcagcccccagcacgtcaccatcagggactacggtgtgtcctggtaccagcagcgggcaggcagtgcccctcgatatctcctctactaccgctcggaggaggatcaccaccggcctgctgacatccccgatcgattctcggcagccaaggatgaggcccacaatgcctgtgtcctcaccattagtcccgtgcagcctgaagacgacgcggattactactgctctgttggctacggctttagtccctaggggtggggtgtgagatgggtgcctcccctctgcctcccatttctgcccctgaccttgggtcccttttaaactttctctgagccttgcttcccctctgtaaaatgggttaataatattcaacatgtcaacaacaaaaaaaaaaaaaaaaaa

(19) United States
(12) Patent Application Publication
(10) Pub. No.: US 2019/0323065 A1
(43) Pub. Date: Oct. 24, 2019

(54) Method of Detecting Active Tuberculosis Using Minimal Gene Signature

(71) Applicant: Imperial Innovations Ltd., London (GB)

(72) Inventors: Michael Levin, London (GB); Myrsini Kafarou, London (GB); Lachlan Coin, London (GB)

(73) Assignee: Imperial Innovations Ltd., London (GB)

(21) Appl. No.: 16/326,828

(22) PCT Filed: Aug. 23, 2017
(86) PCT No.: PCT/EP2017/071196

§ 371 Date: Feb. 20, 2019

(30) Foreign Application Priority Data
Aug. 23, 2016 (GB) 1614394.3

(51) Int. Cl. C12Q 1/689; C12Q 1/6853; C12Q 1/6851; C12Q 1/6837

(52) U.S. Cl. CPC C12Q 1/689; C12Q 1/6853; C12Q 1/6851; C12Q 2600/166; C12Q 2600/158; C12Q 2600/16; C12Q 2600/112; C12Q 1/6837

(57) ABSTRACT

A method of detecting active TB in the presence of a complicating factor, for example, latent TB and/or co-morbidities, such as those that present similar symptoms to TB. The disclosure also relates to a minimal gene signature employed in the said method and to a bespoke gene chip for use in the method. The disclosure further relates to use of gene chips and primer sets in the methods of the disclosure and kits comprising the elements required for performing the method. The disclosure also relates to use of the method to provide a composite expression score which can be used in the diagnosis of TB, particularly in a low resource setting.

The present disclosure relates to a method of detecting active TB in the presence of a complicating factor, for example, latent TB and/or co-morbidities, such as those that present similar symptoms to TB. The disclosure also relates to a minimal gene signature employed in the said method and to a bespoke gene chip for use in the method. The disclosure further relates to use of gene chips and primer sets in the methods of the disclosure and kits comprising the elements required for performing the method. The disclosure also relates to use of the method to provide a composite expression score which can be used in the diagnosis of TB, particularly in a low resource setting.

### BACKGROUND

An estimated 8.8 million new cases and 1.45 million deaths are caused by Tuberculosis, TB (short for tubercle bacillus) each year (World Health Organisation statistics 2011). TB is an infectious disease caused by various species of mycobacteria, typically Mycobacterium tuberculosis. Tuberculosis usually attacks the lungs but can also affect other parts of the body. It is spread through the air when people who have an active TB infection cough, sneeze, or otherwise transmit their saliva. Most infections in humans result in an asymptomatic, latent infection, and about one in ten latent infections eventually progress to active disease, which, if left untreated, kills more than 50% of those infected. Immunosuppression and malnutrition are among the risk factors for developing active TB.

The classic symptoms are a chronic cough with blood-tinged sputum, fever, night sweats, and weight loss (the latter giving rise to the formerly prevalent colloquial term “consumption”). Infection of organs other than the lungs causes a wide range of symptoms. Treatment is difficult and requires long courses of multiple antibiotics. Antibiotic resistance is a growing problem with numbers of multi-drug-resistant tuberculosis cases on the rise. This is, in part, due to the length of treatment needed. Those infected with latent TB are typically asymptomatic and therefore either forget or decided not to take antibiotics. Those infected with active TB often cease treatment when the symptoms clear even though the infection remains.

Correct diagnosis is of utmost importance in the treatment of TB. The treatment regimens for active TB and latent TB are different and so it is important to diagnose the two conditions correctly in order to provide appropriate therapy.

Diagnosis of TB is particularly complicated as it cannot solely be based on symptoms. This is for two reasons: those infected with latent TB exhibit no symptoms and active TB may present similar symptoms to other infections or illnesses. Matters may be further complicated by the fact that TB may not be the only infection or illness that the patient has. Co-morbidities and co-infections often mask the symptoms of active TB and thus the latter goes undiagnosed and untreated. If active TB goes untreated the patient has a high probability of death due to the disease. Not only does TB present similar symptoms to other infectious or non-infectious conditions but it also presents similar radiological features. Thus, identifying the presence of TB definitively can be difficult.

Diagnosis is therefore multi-facetted, relying on clinical and radiological features (commonly chest X-rays), sputum microscopy (with or without culture), tuberculin skin test (TST), blood tests, as well as microscopic examination and microbiological culture of bodily fluids. In many places, such as Africa, which often do not have the resources needed to make a full diagnosis, this is a major impediment to tuberculosis treatment and control. Culture facilities are largely unavailable for TB diagnosis in most African hospitals.

All of the known methods of diagnosis have drawbacks, particularly in HIV co-infected persons in whom radiological features are often atypical:
Sputum microscopy often has low sensitivity in HIV infected patients with TB because cavitatory lung disease is less common in this group, resulting in sputum negative microscopy (Schultz 2010).
Tuberculin skin testing (TST) and Interferon Gamma Release Assays (IGRA) do not discriminate TB from latent TB infection (LTBI) and are of limited utility in African countries where LTBI is highly prevalent in the healthy population. In 2010 Metcalfe et al concluded that neither TST nor IGRA have value for active tuberculosis diagnosis in the context of HIV co-infection in low and middle income countries.
Although molecular diagnosis has improved detection of M. tuberculosis DNA in sputum, the sensitivity of this approach is lower in smear negative samples, even if culture positive, and the method does not detect solely extra-pulmonary disease.

Consequently, a high proportion of active TB cases in sub-Saharan Africa remain undiagnosed, and post-mortem studies show TB to be a frequent, undiagnosed cause of death. Thus, there is an urgent need for improved diagnostic tests for TB, particularly in patients co-infected with HIV.

To meet this need, the present inventors previously developed a method for detecting active TB in a subject derived sample in the presence of a complicating factor, involving testing the expression levels in the genes within 3 different gene signatures. See WO2014/019977, the entire contents of which are incorporated herein by reference. They successfully devised a 27 gene signature for discriminating active TB from latent TB, a 44 gene signature for discriminating active TB from other diseases and a 53 gene signature for discriminating active TB from latent TB and other diseases. These gene signatures were demonstrated to detect active TB with a high degree of specificity and sensitivity.

However, despite the potential of these gene signatures, there is a need to further reduce the number of genes to be tested in the gene signatures, in order to further reduce costs, labour and time taken to analyse and obtain the test results especially in resource poor settings, such as remote villages in sub-Saharan Africa.

### SUMMARY OF THE INVENTION

Accordingly, the present disclosure provides a method for detecting active tuberculosis (TB) in the presence of a complicating factor in a subject derived sample, comprising the step of detecting modulation in gene expression data, generated from RNA levels in the sample, of the genes in a signature selected from the group consisting of:
a) a 3 gene signature comprising FCGR1A, ZNF296 and C1QB for discriminating active TB from latent TB infection;
b) a 6 gene signature comprising GBP6, TMCC1, PRDM1, ARG1, CREB5 and VPREB3 for discriminating active TB from other diseases; and
c) a combination of signatures a) and b).

Advantageously the present inventors developed a novel in-house analysis method called Forward Selection—Partial Least Squares (FS-PLS) and have used it to drastically reduce the original 44 gene signature to a 6 gene signature and the 27 gene signature to a 3 gene signature. They were further able to show that the 6 and 3 gene signatures were capable of detecting active TB with discriminatory power comparable to the original 44 and 27 gene signatures. Accordingly, the presently disclosed method provides the skilled person with the flexibility of using either original 44 gene or 27 gene signatures when a higher sensitivity/specificity is required or the reduced 6 or 3 gene signatures when a reduced number of genes to be tested is desirable.

In one embodiment, the complicating factor is the presence of a co-morbidity, for example wherein the co-morbidity is selected from malignancy, HIV, malaria, pneumonia, Lower Respiratory Tract Infection, Pneumocystis Jirovecii Pneumonia, pelvic inflammatory disease, Urinary Tract Infection, bacterial or viral meningitis, hepatobiliary disease, cryptococcal meningitis, non-TB pleural effusion, empyema, gastroenteritis, peritonitis, gastric ulcer and gastritis.

In one embodiment, wherein the co-morbidity is HIV.

In one embodiment 3 genes in the 6 gene signature are up-regulated, for example wherein the genes PRDM1, GBP6 and CREB5 are up-regulated.

In one embodiment the remaining genes in the 6 gene signature are down-regulated, for example wherein the genes VPREB3, ARG1 and TMCC1 are down-regulated.

In one embodiment 2 genes in the 3 gene signature are up-regulated, for example wherein the gene FCGR1A and C1QB are up-regulated.

In one embodiment the remaining genes in the 3 gene signature are down-regulated, for example wherein the gene ZNF296 is down-regulated.

In one embodiment the method further comprises the steps of:
a. optionally normalising and/or scaling numeric values of the modulation,
b. taking the normalised and/or scaled numeric values or the raw numeric values, each of which comprise both positive and/or negative numeric values and designating all said numeric values to be negative or alternatively all positive,
c. optionally refining the discriminatory power of one or more up-regulated genes and down-regulated genes by statistically weighting some of the numeric values associated therewith, and
d. summating the positive or negative numeric values obtained from step b) or step c) to provide a composite expression score,
   wherein the composite expression score obtained from step d) is compared to a control and the comparison allows the sample to be designated as positive or negative for the relevant infection.

In one embodiment the gene signature further incorporates one or more such as 1, 2, 3, 4, or 5 housekeeping genes.

In one embodiment a patient derived sample is employed in the method.

In one embodiment the detection of gene expression modulation employs a microarray.

In one embodiment the detection of gene expression modulation employs PCR, such as RT-PCR.

In one embodiment the PCR is a multiplex PCR.

In one embodiment the PCR is quantitative.

In one embodiment primers employed in the PCR comprise a label or a combination of labels.

In one embodiment the label is fluorescent or coloured, for example coloured beads.

In one embodiment the detection of gene expression modulation employs a dual colour reverse transcriptase multiplex ligation dependent probe amplification.

In one embodiment the gene expression modulation is detected by employing fluorescence spectroscopy.

In one embodiment the gene expression modulation is detected by employing colourimetric analysis.

In one embodiment the gene expression modulation is detected by employing impedance spectroscopy.

In one embodiment the method comprises the further step of prescribing a treatment for the subject based on the results of the analysis of said gene signature.

In one embodiment the treatment is a treatment for active TB.

In one aspect, there is provided a set of primers for use in multiplex PCR wherein the set of primers includes nucleic acid sequences specific to a polynucleotide gene transcript for at least one gene from the group consisting of:
FCGR1A, ZNF296 and C1QB; and optionally includes nucleic acid sequences specific to a polynucleotide gene transcript for one or more genes selected from the group consisting of: GBP6, TMCC1, PRDM1, ARG1, CREB5 and VPREB3.

In one embodiment the nucleic acid sequences in the set are for no more than a total of 6 genes, such as 2, 3, 4, 5, or 6 genes.

In one embodiment the set of primers, further comprises primers specific to one or more such as 1, 2, 3, 4, or 5 housekeeping genes.

In one embodiment the gene transcript is RNA, for example mRNA.

In one embodiment the primers for each gene are at least a pair of nucleic acid primer sequences.

In one embodiment the primer length is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 bases in length.

In one embodiment at least one primer for each gene comprises a label.

In one embodiment the labels on the primers are independently selected from selected from a fluorescent label, a coloured label, and antibody, step tag, his tag.

In one embodiment each primer in a given pair of primers is labelled, for example where one label quenches the fluorescence of the other label when said labels are within proximity of each other.

In one embodiment the primers are specific to a sequence given in any one of SEQ ID NOs: 1 to 16.

In one aspect, there is provided a point of care test for identifying active TB in a subject comprising the set of primers as described above.

In one aspect, there is provided the use of the set of primers described above in an assay to detect active TB infection in a sample, for example a blood sample.

### BRIEF DESCRIPTION OF THE FIGURES

FIGS. 1A and B—Correlation plots of FS-PLS, Elastic Net and Lasso on the two INTERMAP Metabolomics datasets

Black indicates a correlation coefficient of 1 whilst medium grey indicates a correlation coefficient of −1. FS-PLS selects uncorrelated predictors as indicated by the blue diagonals compared to lasso and elastic net that bring in the model correlated variables.

FIG. 1—Simulation results

Boxplots for RMSE/AUC/ACC (FIG. 2A) and boxplots for number of variables selected (FIG. 2B) for continuous outputs (a, e, i); discrete outputs with 2 classes (b, f, j); discrete outputs with 3 classes (c, g, k); and discrete outputs with 3 classes (d, h, l).

FIG. 3—Reduction of original 27 and 44 gene signatures to minimal 3 and 6 gene signatures

Overview of Example 2 depicting the reduction of the original 27 and 44 gene signatures, derived using Elastic Net, to the new minimal 3 and 6 gene signatures by using FS-PLS.

FIG. 4—Correlation plots of FS-PLS and Elastic Net on the TB datasets

Black indicates correlation coefficient of 1 whilst medium grey correlation represents a coefficient of −1. FS-PLS selects uncorrelated predictors as indicated by the blue diagonals compared to lasso and elastic net that bring in the model correlated variables.

FIG. 5—Comparison of Receiver Operator Curves for 27 gene signature vs 3 gene signature 27 gene signature (Elastic Net) and 3 gene signature (FS-PLS) applied to training cohort [80% of subjects from South African/Malawi HIV+/−patient group described in Kaforou et al (26)], test cohort [20% of subjects from South African/Malawi HIV+/−patient group) and Berry et al dataset (Nature 2010).

### DETAILED DESCRIPTION

In one embodiment of the present disclosure the gene signature is the minimum set of genes required to optimally detect the infection or discriminate the disease.

Optimally is intended to mean the smallest set of genes needed to detect active TB without significant loss of specificity and/or sensitivity of the signature's ability to detect or discriminate.

Detect or detecting as employed herein is intended to refer to the process of identifying an active TB infection in a sample, in particular through detecting modulation of the relevant genes in the signature.

Discriminate refers to the ability of the signature to differentiate between different disease status, for example latent and active TB. Detect and discriminate are interchangeable in the context of the gene signature.

In one embodiment the method is able to detect an active TB infection in a sample.

Subject as employed herein is a human suspected of TB infection from whom a sample is derived. The term patient may be used interchangeably although in one embodiment a patient has a morbidity.

In one embodiment the subject is an adult. Adult is defined herein as a person of 18 years of age or older.

In one embodiment the subject is a child. Child as employed herein refers to a person under the age of 18, such as 5 to 17 years of age.

Modulation of gene expression as employed herein means up-regulation or down-regulation of a gene or genes.

Up-regulated as employed herein is intended to refer to a gene transcript which is expressed at higher levels in a diseased or infected patient sample relative to, for example, a control sample free from a relevant disease or infection, or in a sample with latent disease or infection or a different stage of the disease or infection, as appropriate.

Down-regulated as employed herein is intended to refer to a gene transcript which is expressed at lower levels in a diseased or infected patient sample relative to, for example, a control sample free from a relevant disease or infection or in a sample with latent disease or infection or a different stage of the disease or infection.

The modulation is measured by measuring levels of gene expression by an appropriate technique. Gene expression as employed herein is the process by which information from a gene is used in the synthesis of a functional gene product. These products are often proteins, but in non-protein coding genes such as ribosomal RNA (rRNA), transfer RNA (tRNA) or small nuclear RNA (snRNA) genes, the product is a functional RNA. That is to say, RNA with a function.

Gene expression data as employed herein is intended to refer to any data generated from a patient sample that is indicative of the expression of the two or more genes, for example 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50.

A complicating factor as employed herein refers to at least one clinical status or at least one medical condition that would generally render it more difficult to identify the presence of active TB in the sample, for example a latent TB infection or a co-morbidity.

Co-morbidity as employed herein refers the presence of one or more disorders or diseases in addition to TB, for example malignancy such as cancer or co-infection. Co-morbidity may or may not be endemic in the general population.

In one embodiment the co-morbidity is a co-infection.

Co-infection as employed herein refers to bacterial infection, viral infection such as HIV, fungal infection and/or parasitic infection such as malaria. HIV infection as employed herein also extends to include AIDS.

In one embodiment other disease (OD) is a co-morbidity.

In one embodiment the 6 gene signature comprising GBP6, TMCC1, PRDM1, ARG1, CREB5 and VPREB3 is able to detect active TB in the presence of a co-morbidity such as a co-infection and is able to discriminate active TB from other diseases. This is despite the increased inflammatory response of the patient to said other infection.

In one embodiment co-morbidity is selected from malignancy, HIV, malaria, pneumonia, Lower Respiratory Tract Infection, Pneumocystis Jirovecii Pneumonia, pelvic inflammatory disease, Urinary Tract Infection, bacterial or viral meningitis, hepatobiliary disease, cryptococcal meningitis, non-TB pleural effusion, empyema, gastroenteritis, peritonitis, gastric ulcer and gastritis. In one embodiment malignancy is a neoplasia, such as bronchial carcinoma, lymphoma, cervical carcinoma ovarian carcinoma, mesothelioma, gastric carcinoma, metastatic carcinoma, benign salivary tumour, dermatological tumour or Kaposi's sarcoma.

The 3 gene signature comprising FCGR1A, ZNF296 and C1QB is useful in discriminating active TB infection from latent TB infection.

Active TB as employed herein refers to a person who is infected with TB which is not latent.

In one embodiment active TB is where the disease is progressing as opposed to where the disease is latent.

In one embodiment a person with active TB is capable of spreading the infection to others.

In one embodiment a person with active TB has one or more of the following: a skin test or blood test result indicating TB infection, an abnormal chest x-ray, a positive sputum smear or culture, active TB bacteria in his/her body, feels sick and may have symptoms such as coughing, fever, and weight loss.

In one embodiment a person with active TB has one or more of the following symptoms: coughing, bloody sputum, fever and/or weight loss.

In one embodiment the active TB infection is pulmonary and/or extra-pulmonary.

Pulmonary as employed herein refers to an infection in the lungs.

Extra-pulmonary as employed herein refers to infection outside the lungs, for example, infection in the pleura, infection in the lymphatic system, infection in the central nervous system, infection in the genito-urinary tract, infection in the bones, infection in the brain and/or infection in the kidneys.

Symptoms of pulmonary TB include: a persistent cough that brings up thick phlegm, which may be bloody; breathlessness, which is usually mild to begin with and gradually gets worse; weight loss; lack of appetite; a high temperature of 38° C. (100.4° F.) or above; extreme tiredness; and a sense of feeling unwell.

Symptoms of lymph node TB include: persistent, painless swelling of the lymph nodes, which usually affects nodes in the neck, but swelling can occur in nodes throughout your body; over time, the swollen nodes can begin to release a discharge of fluid through the skin.

Symptoms of skeletal TB include: bone pain; curving of the affected bone or joint; loss of movement or feeling in the affected bone or joint and weakened bone that may fracture easily.

Symptoms of gastrointestinal TB include: abdominal pain; diarrhoea and anal bleeding.

Symptoms of genitourinary TB include: a burning sensation when urinating; blood in the urine; a frequent urge to pass urine during the night and groin pain.

Symptoms of central nervous system TB include: headaches; being sick; stiff neck; changes in your mental state, such as confusion; blurred vision and fits.

Latent TB as employed herein refers to a subject who is infected with TB but is asymptomatic. A sputum test will generally be negative and the infection cannot be spread to others.

In one embodiment a person with latent TB infection has one of more of the following: a skin test or blood test result indicating TB infection, a normal chest x-ray and a negative sputum test, TB bacteria in his/her body that are alive, but inactive, does not feel sick, cannot spread TB bacteria to others

In one embodiment a person with latent TB needs treatment to prevent TB disease becoming active.

In one embodiment the method of the present disclosure is able to differentiate TB from different conditions/diseases or infections which have similar clinical symptoms.

Similar symptoms as employed herein includes one or more symptoms from pulmonary TB, lymph node TB, skeletal TB, gastrointestinal TB, genitourinary TB and/or central nervous system TB.

In one embodiment the method according to the present disclosure is performed on a subject with acute infection.

In a further embodiment the sample is a subject sample from a febrile subject, that is to say a subject with a temperature above the normal body temperature of 37.5° C.

In one embodiment the genes employed have identity with genes listed in the relevant tables, such as Table 3 and 4.

In one embodiment the 6 gene signature comprises or consists of at least up-regulated genes PRDM1, GBP6 and CREB5.

In one embodiment the 6 gene signature comprises or consists of at least down-regulated genes VPREB3, ARG1 and TMCC1.

In one embodiment the 6 gene signature comprises or consists of at least up-regulated genes PRDM1, GBP6 and CREB5, and down-regulated genes VPREB3, ARG1 and TMCC1.

In one embodiment the 3 gene signature comprises or consists of at least up-regulated genes FCGR1A and C1QB.

In one embodiment the 3 gene signature comprises or consists of at least down-regulated gene ZNF296.

In one embodiment 3 gene signature comprises or consists of at least up-regulated genes FCGR1A and C1QB and down-regulated gene ZNF296.

In one embodiment the 3 and 6 gene signatures are tested in parallel.

In one embodiment one or more, for example 1 to 21, such as 2, 3, 4, 5, 6, 7, 8, 9, 10, 11, 12, 13, 14, 15, 16, 17, 18, 19 or 20, genes are replaced by a gene with an equivalent function provided the signature retains the ability to detect/discriminate the relevant clinical status without significant loss in specificity and/or sensitivity.

In one embodiment the gene signature is based on two genes of primary importance. Of primary importance as used herein means that the gene expression levels of the two genes is representative of the gene expression levels of other genes. For example, the expression levels of the first gene of primary importance may be highly correlated with the expression levels of a first group of genes, whilst the expression levels of the second gene of primary importance may be highly correlated with the expression levels of a second group of genes.

Therefore, each gene of primary importance may be used as a representative of the other highly correlated genes from their respective groups, thereby eliminating the need to test all of the genes within each group. In other words, testing the expression levels of just the two genes of primary importance provides a similar sensitivity and/or specificity as testing the expression levels of all of the genes.

In one embodiment each of the genes in the 3, 6 gene signatures is significantly differentially expressed in the sample with active TB compared to a comparator group.

Significantly differentially expressed as employed herein means the sample with active TB shows a log 2 fold change >0.5 compared to the comparator group.

In one embodiment, in the 3 gene signature the comparator group is LTBI.

In one embodiment, in the 6 gene signature the comparator group is a person with “other disease” (OD), that is a disease that is not active TB but has similar symptoms. “Presented in the form of” as employed herein refers to the laying down of genes from one or more of the signatures in the form of probes on a microarray.

Accurately and robustly as employed herein refers to the fact that the method can be employed in a practical setting, such as Africa, and that the results of performing the method properly give a high level of confidence that a true result is obtained.

High confidence is provided by the method when it provides few results that are false positives (i.e. the result suggests that the subject has active TB when they do not) and also has few false negatives (i.e. the result suggest that the subject does not have active TB when they do).

High confidence would include 90% or greater confidence, such as 91, 92, 93, 94, 95, 96, 97, 98, 99 or 100% confidence when an appropriate statistical test is employed.

In one embodiment the method provides a sensitivity of 80% or greater such as 90% or greater in particular 95% or greater, for example where the sensitivity is calculated as below:

$$sensitivity = \frac{number of true positives}{number of true positives + number of false negatives} = probability of a positive test given that the patient is ill$$

In one embodiment the method provides a high level of specificity, for example 80% or greater such as 90% or greater in particular 95% or greater, for example where specificity is calculated as shown below:

$$specificity = \frac{number of true negatives}{number of true negatives + number of false positives} = probability of a negative test given that the patient is well$$

In one embodiment the sensitivity of method of the 3 gene signature is 85 to 100%, such as 84, 85, 86, 87, 88, 89, 90, 91, 92, 93, 94, 95, 96, 97, 98 or 99%.

In one embodiment the specificity of the method of the 3 gene signature is 85 to 100%, such as 84, 85, 86, 87, 88, 89, 90, 91, 92, 93, 94, 95, 96, 97, 98 or 99%.

In one embodiment the sensitivity of the method of the 6 gene signature is 85 to 100%, such as 84, 85, 86, 87, 88, 89, 90, 91, 92, 93, 94, 95, 96, 97, 98 or 99%.

In one embodiment the specificity of the method of the 6 gene signature is 85 to 100%, such as 84, 85, 86, 87, 88, 89, 90, 91, 92, 93, 94, 95, 96, 97, 98 or 99%.

Thus in one embodiment DNA or RNA, in particular mRNA from the subject sample is analysed. In one embodiment the sample is solid or fluid, for example blood or serum or a processed form of any one of the same.

A fluid sample as employed herein refers to liquids originating from inside the bodies of living people. They include fluids that are excreted or secreted from the body as well as body water that normally is not. Includes amniotic fluid, aqueous humour and vitreous humour, bile, blood serum, breast milk, cerebrospinal fluid, cerumen (earwax), chyle, endolymph and perilymph, gastric juice, mucus (including nasal drainage and phlegm), sputum, peritoneal fluid, pleural fluid, saliva, sebum (skin oil), semen, sweat, tears, vaginal secretion, vomit, urine. Particularly blood and serum.

Blood as employed herein refers to whole blood, that is serum, blood cells and clotting factors, typically peripheral whole blood.

Serum as employed herein refers to the component of whole blood that is not blood cells or clotting factors. It is plasma with fibrinogens removed.

In one embodiment the subject derived sample is a blood sample.

In one or more embodiments the analysis is ex vivo.

In one embodiment the sample is whole blood. Hence in one embodiment the RNA sample is derived from whole blood.

The RNA sample may be subjected to further amplification by PCR, such as whole genome amplification in order to increase the amount of starting RNA template available for analysis.

Alternatively, the RNA sample may be converted into cDNA by reverse transcriptase, such as HIV-1 reverse transcriptase, moloney murine leukaemia virus (M-MLV) reverse transcriptase, AMV reverse transcriptase and telomersease reverse transcriptase. Such amplification steps may be necessary for smaller sample volumes, such as blood samples obtained from children.

Ex vivo as employed herein means that which takes place outside the body.

There are a number of ways in which gene expression can be measured including microarrays, tiling arrays, DNA or RNA arrays for example on gene chips, RNA-seq and serial analysis of gene expression.

Any suitable method of measuring gene modulation may be employed in the method of the present disclosure.

Polymerase chain reaction (PCR) as employed herein refers to a widely used molecular technique to make multiple copies of a target DNA sequence. The method relies on thermal cycling, consisting of cycles of repeated heating and cooling of the reaction for DNA melting and enzymatic replication of the DNA. Primers containing sequences complementary to the target region along with a DNA polymerase, which the method is named after, are key components to enable selective and repeated amplification. As PCR progresses, the DNA generated is itself used as a template for replication, setting in motion a chain reaction in which the DNA template is exponentially amplified.

Multiplex PCR as employed herein refers to the use of a polymerase chain reaction (PCR) to amplify two or more different DNA sequences simultaneously, i.e. as if performing many separate PCR reactions together in one reaction.

Primer as employed herein is intended to refer to a short strand of nucleic acid sequence, usually a chemically synthesised oligonucleotide, which serve as a starting point for DNA synthesis reactions. Primers are typically about 15 base pairs long but can vary from 5 to 100 bases long. It is required in processes such as PCR because DNA polymerases can only add new nucleotides or base pairs to an existing strand of DNA. During a PCR reaction, the primer hybridises to its complementary sequence in a DNA sample. Next, DNA polymerase starts replication at the 3′-end of the primer and extends the primer by copying the sequence of the opposite DNA strand.

In one embodiment the primers of the present disclosure are specific for RNA, such as mRNA, i.e. they are complementary to RNA sequences. In another embodiment, the primers are specific for cDNA, i.e. they are complementary to cDNA sequences.

In one embodiment the primers of the present disclosure comprise a label which enables the primers to be detected or isolated. Examples of labels include but are not limited to a fluorescent label, a coloured label, and antibody, step tag, his tag.

In another embodiment, each primer in a given pair of primers is labelled, for example where one label (also known as a quencher) quenches the fluorescence of the other label when said labels are within proximity of each other. Such labels are particularly useful in real time PCR reactions for example. Examples of such label pairs include 6-carboxyfluorescein (FAM) and tetrachlorofluorescein, or tetramethylrhodamine and tetrachlorofluorescein.

Point of care test or bedside test as used herein is intended to refer to a medical diagnostic test which is conducted at or near the point of care, i.e. at the time and place of patient care. This is in contrast with a conventional diagnostic test which is typically confined to the medical laboratory and involves sending specimens away from the point of care to the laboratory for testing. Such diagnostic tests often require many hours or days before the results of the test can be received. In the meantime, patient care must continue without knowledge of the test results. In comparison, a point of care test is typically a simple medical test that can be performed rapidly.

In one embodiment the gene expression data is generated from a microarray, such as a gene chip.

In one aspect of the disclosure there is provided a gene chip comprising one or more of the gene signatures selected from the group consisting of:

a) a 3 gene signature comprising FCGR1A, ZNF296 and C1QB;

b) a 6 gene signature comprising GBP6, TMCC1, PRDM1, ARG1, CREB5 and VPREB36; and

optionally;

c) one or more house-keeping genes.

In a further aspect the present disclosure includes use of a known or commercially available gene chip in the method of the present disclosure.

Advantageously the different expression patterns represented by the gene signatures employed in the method of the present disclosure correlate across geographic location and HIV infected status (i.e. positive or negative). That is to say, the method is applicable to different geographic locations regardless of the presence or absence of HIV.

Microarray as employed herein includes RNA or DNA arrays, such as mRNA arrays.

A gene chip is essentially a microarray that is to say an array of discrete regions, typically nucleic acids, which are separate from one another and are, for example arrayed at a density of between, about 100/cm² to 1000/cm², but can be arrayed at greater densities such as 10000/cm².

The principle of a microarray experiment, is that mRNA from a given cell line or tissue is used to generate a labelled sample typically labelled cDNA or cRNA, termed the ‘target’, which is hybridised in parallel to a large number of, nucleic acid sequences, typically DNA or RNA sequences, immobilised on a solid surface in an ordered array. Tens of thousands of transcript species can be detected and quantified simultaneously. Although many different microarray systems have been developed the most commonly used systems today can be divided into two groups.

Using this technique, arrays consisting of more than 30,000 cDNAs can be fitted onto the surface of a conventional microscope slide. For oligonucleotide arrays, short 20-25mers are synthesised in situ, either by photolithography onto silicon wafers (high-density-oligonucleotide arrays from Affymetrix) or by ink-jet technology (developed by Rosetta Inpharmatics and licensed to Agilent Technologies).

Alternatively, pre-synthesised oligonucleotides can be printed onto glass slides. Methods based on synthetic oligonucleotides offer the advantage that because sequence information alone is sufficient to generate the DNA to be arrayed, no time-consuming handling of cDNA resources is required. Also, probes can be designed to represent the most unique part of a given transcript, making the detection of closely related genes or splice variants possible. Although short oligonucleotides may result in less specific hybridization and reduced sensitivity, the arraying of pre-synthesised longer oligonucleotides (50-100 mers) has recently been developed to counteract these disadvantages.

In one embodiment the gene chip is an off the shelf, commercially available chip, for example HumanHT-12 v4 Expression BeadChip Kit, available from Illumina, NimbleGen microarrays from Roche, Agilent, Eppendorf and Genechips from Affymetrix such as HU-UI 33.Plus 2.0 gene chips.

In an alternate embodiment the gene chip employed in the present invention is a bespoke gene chip, that is to say the chip contains only the target genes which are relevant to the desired profile. Custom made chips can be purchased from companies such as Roche, Affymetrix and the like. In yet a further embodiment the bespoke gene chip comprises a minimal disease specific transcript set.

In one embodiment the chip comprises or consists of the genes in the 6 gene signature comprising GBP6, TMCC1, PRDM1, ARG1, CREB5 and VPREB36.

In one embodiment the chip comprises or consists of the genes in the 3 gene signature comprising FCGR1A, ZNF296 and C1QB.

In one embodiment the chip comprises or consists of the genes in the 6 gene signature in combination with the genes in the 3 gene signature.

In one embodiment the following Illumina transcript ID number probes are used to detect the modulation in gene expression levels: ILMN_2176063 for FCGR1A, ILMN_1693242 for ZNF296, ILMN_1796409 for C1QB, ILMN_2294784 for PRDM1, ILMN_1756953 for GBP6, ILMN_1728677 for CREB5, ILMN_1700147 for VPREB3, ILMN_1812281 for ARG1 and ILMN_1677963 for TMCC1. In one or more embodiments above the chip may further include 1 or more, such as 1 to 10, house-keeping genes.

In one embodiment the gene expression data is generated in solution using appropriate probes for the relevant genes.

Probe as employed herein is intended to refer to a hybridisation probe which is a fragment of DNA or RNA of variable length (usually 100-1000 bases long) which is used in DNA or RNA samples to detect the presence of nucleotide sequences (the DNA target) that are complementary to the sequence in the probe. The probe thereby hybridises to single-stranded nucleic acid (DNA or RNA) whose base sequence allows probe-target base pairing due to complementarity between the probe and target.

In one embodiment the method according to the present disclosure and for example chips employed therein may comprise one or more house-keeping genes. House-keeping genes as employed herein is intended to refer to genes that are not directly relevant to the profile for identifying the disease or infection but are useful for statistical purposes and/or quality control purposes, for example they may assist with normalising the data, in particular a house-keeping gene is a constitutive gene i.e. one that is transcribed at a relatively constant level. The housekeeping gene's products are typically needed for maintenance of the cell.

Examples of housekeeping genes include but are not limited to actin, GAPDH, ubiquitin, 18s rRNA, RPII (POLR2A), TBP, PPIA, GUSB, HSPCB, YWHAZ, SDHA, RPS13, HPRT1 and B4GALT6.

In one embodiment minimal disease specific transcript set as employed herein means the minimum number of genes need to robustly identify the target disease state.

Minimal discriminatory gene set is interchangeable with minimal disease specific transcript set.

Normalising as employed herein is intended to refer to statistically accounting for background noise by comparison of data to control data, such as the level of fluorescence of house-keeping genes, for example fluorescent scanned data may be normalized using RMA to allow comparisons between individual chips. Irizarry et al 2003 describes this method.

Scaling as employed herein refers to boosting the contribution of specific genes which are expressed at low levels or have a high fold change but still relatively low fluorescence such that their contribution to the diagnostic signature is increased.

Fold change is often used in analysis of gene expression data in microarray and RNA-Seq experiments, for measuring change in the expression level of a gene and is calculated simply as the ratio of the final value to the initial value i.e. if the initial value is A and final value is B, the fold change is B/A. Tusher et al 2001.

In programs such as Arrayminer, fold change of gene expression can be calculated. The statistical value attached to the fold change is calculated and is the more significant in genes where the level of expression is less variable between subjects in different groups and, for example where the difference between groups is larger.

The step of obtaining a suitable sample from the subject is a routine technique, which involves taking a blood sample. This process presents little risk to donors and does not need to be performed by a doctor but can be performed by appropriately trained support staff. In one embodiment the sample derived from the subject is approximately 2.5 ml of blood, however smaller volumes can be used for example 0.5-1 ml.

Blood or other tissue fluids are immediately placed in an RNA stabilizing buffer such as included in the Pax gene tubes, or Tempus tubes.

If storage is required then it should usually be frozen within 3 hours of collections at −80° C.

In one embodiment the gene expression data is generated from RNA levels in the sample.

For microarray analysis the blood may be processed using a suitable product, such as PAX gene blood RNA extraction kits (Qiagen).

Total RNA may also be purified using the Tripure method—Tripure extraction (Roche Cat. No. 1 667 165). The manufacturer's protocols may be followed. This purification may then be followed by the use of an RNeasy Mini kit—clean-up protocol with DNAse treatment (Qiagen Cat. No. 74106).

Quantification of RNA may be completed using optical density at 260 nm and Quant-IT RiboGreen RNA assay kit (Invitrogen—Molecular probes RI 1490). The Quality of the 28s and 18s ribosomal RNA peaks can be assessed by use of the Agilent bioanalyser.

In another embodiment the method further comprises the step of amplifying the RNA. Amplification may be performed using a suitable kit, for example TotalPrep RNA Amplification kits (Applied Biosystems).

In one embodiment an amplification method may be used in conjunction with the labelling of the RNA for microarray analysis. The Nugen 3′ ovation biotin kit (Cat: 2300-12, 2300-60).

The RNA derived from the subject sample is then hybridised to the relevant probes, for example which may be located on a chip. After hybridisation and washing, where appropriate, analysis with an appropriate instrument is performed.

In performing an analysis to ascertain whether a subject presents a gene signature indicative of disease or infection according to the present disclosure, the following steps are performed: obtain mRNA from the sample and prepare nucleic acids targets, hybridise to the array under appropriate conditions, typically as suggested by the manufactures of the microarray (suitably stringent hybridisation conditions such as 3×SSC, 0.1% SDS, at 50° C.) to bind corresponding probes on the array, and wash if necessary to remove unbound nucleic acid targets and analyse the results.

In one embodiment the readout from the analysis is fluorescence.

In one embodiment the readout from the analysis is colorimetric.

In one embodiment physical detection methods, such as changes in electrical impedance, nanowire technology or microfluidics may be used.

In one embodiment there is provided a method which further comprises the step of quantifying RNA from the subject sample.

If a quality control step is desired, software such as Genome Studio software may be employed. Numeric value as employed herein is intended to refer to a number obtained for each relevant gene, from the analysis or readout of the gene expression, for example the fluorescence or colorimetric analysis. The numeric value obtained from the initial analysis may be manipulated, corrected and if the result of the processing is a still a number then it will be continue to be a numeric value.

By converting is meant processing of a negative numeric value to make it into a positive value or processing of a positive numeric value to make it into a negative value by simple conversion of a positive sign to a negative or vice versa.

Analysis of the subject-derived sample will for the genes analysed will give a range of numeric values some of which are positive (preceded by + and in mathematical terms considered greater than zero) and some of which are negative (preceded by − and in strict mathematical terms are considered to less than zero). The positive and negative in the context of gene expression analysis is a convenient mechanism for representing genes which are up-regulated and genes which are down regulated.

In the method of the present disclosure either all the numeric values of genes which are down-regulated and represented by a negative number are converted to the corresponding positive number (i.e. by simply changing the sign) for example −1 would be converted to 1 or all the positive numeric values for the up-regulated genes are converted to the corresponding negative number.

The present inventors have established that this step of rendering the numeric values for the gene expressions positive or alternatively all negative allows the summating of the values to obtain a single value that is indicative of the presence of disease or infection or the absence of the same.

This is a huge simplification of the processing of gene expression data and represents a practical step forward thereby rendering the method suitable for routine use in the clinic.

By discriminatory power is meant the ability to distinguish between a TB infected and a non-infected sample (subject) or between active TB infection and other infections (such as HIV) in particular those with similar symptoms or between a latent infection and an active infection.

The discriminatory power of the method according to the present disclosure may, for example, be increased by attaching greater weighting to genes which are more significant in the signature, even if they are expressed at low or lower absolute levels.

As employed herein, raw numeric value is intended to, for example refer to unprocessed fluorescent values from the gene chip, either absolute fluorescence or relative to a house keeping gene or genes.

Summating as employed herein is intended to refer to act or process of adding numerical values.

Composite expression score as employed herein means the sum (aggregate number) of all the individual numerical values generated for the relevant genes by the analysis, for example the sum of the fluorescence data for all the relevant up and down regulated genes. The score may or may not be normalised and/or scaled and/or weighted.

In one embodiment the composite expression score is normalised.

In one embodiment the composite expression score is scaled.

In one embodiment the composite expression score is weighted.

Weighted or statistically weighted as employed herein is intended to refer to the relevant value being adjusted to more appropriately reflect its contribution to the signature.

In one embodiment the method employs a simplified risk score as employed in the examples herein.

Simplified risk score is also known as disease risk score (DRS).

Control as employed herein is intended to refer to a positive (control) sample and/or a negative (control) sample which, for example is used to compare the subject sample to, and/or a numerical value or numerical range which has been defined to allow the subject sample to be designated as positive or negative for disease/infection by reference thereto.

Positive control sample as employed herein is a sample known to be positive for the pathogen or disease in relation to which the analysis is being performed, such as active TB.

Negative control sample as employed herein is intended to refer to a sample known to be negative for the pathogen or disease in relation to which the analysis is being performed.

In one embodiment the control is a sample, for example a positive control sample or a negative control sample, such as a negative control sample.

In one embodiment the control is a numerical value, such as a numerical range, for example a statistically determined range obtained from an adequate sample size defining the cut-offs for accurate distinction of disease cases from controls.

Conversion of Multi-Gene Transcript Disease Signatures into a Single Number Disease Score

Once the RNA expression signature of the disease has been identified by variable selection, the transcripts are separated based on their up- or down-regulation relative to the comparator group. The two groups of transcripts are selected and collated separately.

### Summation of Up-Regulated and Down-Regulated RNA Transcripts

To identify the single disease risk score for any individual patient, the raw intensities, for example fluorescent intensities (either absolute or relative to housekeeping standards) of all the up-regulated RNA transcripts associated with the disease are summated. Similarly summation of all down-regulated transcripts for each individual is achieved by combining the raw values (for example fluorescence) for each transcript relative to the unchanged housekeeping gene standards. Since the transcripts have various levels of expression and respectively their fold changes differ as well, instead of summing the raw expression values, they can be scaled and normalised between 0.1. Alternatively they can be weighted to allow important genes to carry greater effect. Then, for every sample the expression values of the signature's transcripts are summated, separately for the up- and down-regulated transcripts.

The total disease score incorporating the summated fluorescence of up- and down-regulated genes is calculated by adding the summated score of the down-regulated transcripts (after conversion to a positive number) to the summated score of the up-regulated transcripts, to give a single number composite expression score. This score maximally distinguishes the cases and controls and reflects the contribution of the up- and down-regulated transcripts to this distinction.

### Comparison of the Disease Risk Score in Cases and Controls

The composite expression scores for patients and the comparator group may be compared, in order to derive the means and variance of the groups, from which statistical cut-offs are defined for accurate distinction of cases from controls. Using the disease subjects and comparator populations, sensitivities and specificities for the disease risk score may be calculated using, for example a Support Vector Machine and internal elastic net classification.

Disease risk score as employed herein is an indicator of the likelihood that patient has active TB when comparing their composite expression score to the comparator group's composite expression score.

Development of the Disease Risk Score into a Simple Clinical Test for Disease Severity or Disease Risk Prediction

The approach outlined above in which complex RNA expression signatures of disease or disease processes are converted into a single score which predicts disease risk can be used to develop simple, cheap and clinically applicable tests for disease diagnosis or risk prediction.

The procedure is as follows: For tests based on differential gene expression between cases and controls (or between different categories of cases such as severity), the up- and down-regulated transcripts identified as relevant may be printed onto a suitable solid surface such as microarray slide, bead, tube or well.

Up-regulated transcripts may be co-located separately from down-regulated transcripts either in separate wells or separate tubes. A panel of unchanged housekeeping genes may also be printed separately for normalisation of the results.

RNA recovered from individual patients using standard recovery and quantification methods (with or without amplification) is hybridised to the pools of up- and down-regulated transcripts and the unchanged housekeeping transcripts.

Control RNA is hybridised in parallel to the same pools of up- or down-regulated transcripts.

Total value, for example fluorescence for the subject sample and optionally the control sample is then read for up- and down-regulated transcripts and the results combined to give a composite expression score for patients and controls, which is/are then compared with a reference range of a suitable number of healthy controls or comparator subjects.

### Correcting the Detected Signal for the Relative Abundance of RNA Species in the Subject Sample

The details above explain how a complex signature of many transcripts can be reduced to the minimum set that is maximally able to distinguish between patients and other phenotypes. For example, within the up-regulated transcript set, there will be some transcripts that have a total level of expression many fold lower than that of others. However, these transcripts may be highly discriminatory despite their overall low level of expression. The weighting derived from the elastic net coefficient can be included in the test, in a number of different ways. Firstly, the number of copies of individual transcripts included in the assay can be varied. Secondly, in order to ensure that the signal from rare, important transcripts are not swamped by that from transcripts expressed at a higher level, one option would be to select probes for a test that are neither overly strongly nor too weakly expressed, so that the contribution of multiple probes is maximised. Alternatively, it may be possible to adjust the signal from low-abundance transcripts by a scaling factor.

Whilst this can be done at the analysis stage using current transcriptomic technology as each signal is measured separately, in a simple colorimetric test only the total colour change will be measured, and it would not therefore be possible to scale the signal from selected transcripts. This problem can be circumnavigated by reversing the chemistry usually associated with arrays. In conventional array chemistry, the probes are coupled to a solid surface, and the amount of biotin-labelled, patient-derived target that binds is measured. Instead, we propose coupling the biotin-labelled cRNA derived from the patient to an avidin-coated surface, and then adding DNA probes coupled to a chromogenic enzyme via an adaptor system. At the design and manufacturing stage, probes for low-abundance but important transcripts are coupled to greater numbers, or more potent forms of the chromogenic enzyme, allowing the signal for these transcripts to be ‘scaled-up’ within the final single-channel colorimetric readout. This approach would be used to normalise the relative input from each probe in the up-regulated, down-regulated and housekeeping channels of the kit, so that each probe makes an appropriately weighted contribution to the final reading, which may take account of its discriminatory power, suggested by the weights of variable selection methods.

The detection system for measuring multiple up or down regulated genes may also be adapted to use rTPCR to detect the transcripts comprising the diagnostic signature, with summation of the separate pooled values for up and down regulated transcripts, or physical detection methods such as changes in electrical impedance. In this approach, the transcripts in question are printed on nanowire surfaces or within microfluidic cartridges, and binding of the corresponding ligand for each transcript is detected by changes in impedance or other physical detection system

The present disclosure extends to a custom made chip comprising a minimal discriminatory gene set for diagnosis of active TB from other conditions, in particular those with similar symptoms, for example comprising probes specific for the genes in the 6 gene signature and/or 3 gene signature. In one embodiment the gene chip is a fluorescent gene chip that is to say the readout is fluorescence. Fluorescence as employed herein refers to the emission of light by a substance that has absorbed light or other electromagnetic radiation.

Thus in an alternate embodiment the gene chip is a colorimetric gene chip, for example colorimetric gene chip uses microarray technology wherein avidin is used to attach enzymes such as peroxidase or other chromogenic substrates to the biotin probe currently used to attach fluorescent markers to DNA. The present disclosure extends to a microarray chip adapted to read by colorimetric analysis and adapted for the analysis of active TB infection in a patient. The present disclosure also extends to use of a colorimetric chip to analyse a subject sample for active TB infection.

Colorimetric as employed herein refers to as assay wherein the output is in the human visible spectrum.

In an alternative embodiment, a gene set indicative of active TB may be detected by physical detection methods including nanowire technology, changes in electrical impedance, or microfluidics.

The readout for the assay can be converted from a fluorescent readout as used in current microarray technology into a simple colorimetric format or one using physical detection methods such as changes in impedance, which can be read with minimal equipment. For example, this is achieved by utilising the Biotin currently used to attach fluorescent markers to DNA. Biotin has high affinity for avidin which can be used to attach enzymes such as peroxidase or other chromogenic substrates. This process will allow the quantity of cRNA binding to the target transcripts to be quantified using a chromogenic process rather than fluorescence. Simplified assays providing yes/no indications of disease status can then be developed by comparison of the colour intensity of the up- and down-regulated pools of transcripts with control colour standards. Similar approaches can enable detection of multiple gene signatures using physical methods such as changes in electrical impedance.

This aspect of the invention is likely to be particularly advantageous for use in remote or under-resourced settings or for rapid diagnosis in “near patient” tests. For example, places in Africa because the equipment required to read the chip is likely to be simpler.

Multiplex assay as employed herein refers to a type of assay that simultaneously measures several analytes (often dozens or more) in a single run/cycle of the assay. It is distinguished from procedures that measure one analyte at a time.

In one embodiment there is provided a bespoke gene chip for use in the method, in particular as described herein.

In one embodiment there is provided use of a known gene chip for use in the method described herein in particular to identify one or more gene signatures described herein.

In one embodiment there is provided a method of treating latent TB after diagnosis employing the method disclosed herein.

In one embodiment there is provided a method of treating active TB after diagnosis employing the method disclosed herein.

Examples of suitable agents for treating TB include but are not limited to isoniazid, rifampin, ethambutol, pyrazinamide, streptomycin, kanamycin, amikacin, capreomycin, levofloxacin, moxifloxacin, ofloxacin, para-aminosalicylic acid, cycloserine, terizidone, thionamide, protionamide, clofaximine, linezolid, amoxicillin/clavulanate, thioacetazone, imipenem/cilastatin, high dose isoniazid, clarithromycin.

In one embodiment the treatment comprises a combination of two or more of the above agents.

Gene signature, gene set, disease signature, diagnostic signature and gene profile are used interchangeably throughout and should be interpreted to mean gene signature.

In the context of this specification “comprising” is to be interpreted as “including”.

Aspects of the invention comprising certain elements are also intended to extend to alternative embodiments “consisting” or “consisting essentially” of the relevant elements.

Where technically appropriate, embodiments of the invention may be combined.

Embodiments are described herein as comprising certain features/elements. The disclosure also extends to separate embodiments consisting or consisting essentially of said features/elements.

Technical references such as patents and applications are incorporated herein by reference.

Any embodiments specifically and explicitly recited herein may form the basis of a disclaimer either alone or in combination with one or more further embodiments.

### EXAMPLES

### Example 1—Development of Forward Selection—Partial Least Squares (FS-PLS) Method

### Overview of Biomarker Selection Methods in 'Omics Datasets

Conventional methods for variable selection and model building, as applied to omics data, fall broadly into three categories. A comprehensive review on the methodological challenges behind omics-based biomarker selection is given by Hyam and colleagues (2) but for the scope of this paper, we provide a brief description of methodologies with their relative strengths and limitations.

(A) Univariate Variable Selection Followed by Model Fitting.

These methods first rank the variables by applying a univariate test statistic. (ie t-test, Cochran-Armitage test) The top ranked variables are then selected based on a threshold and model fitting is achieved using a machine learning classification method (ie. support vector machines (3), decision trees (4) and Maximum Likelihood Discriminant analysis such as Linear Discriminant Analysis and Diagonal Linear Discriminant Analysis (5). These methods benefit by the prediction power of the classification algorithm but depend highly on the original pre-filtering, which requires a threshold that most of the times is arbitrary and if it is too stringent it might miss important variables or if it is too loose it might include redundant variables.

(B) Multivariate Model Fitting with Embedded Variable Selection.

These methods perform variable selection and model fitting simultaneously. Most regression-based techniques, such as Forward Selection (6, 7)) consider all variables simultaneously and allow each variable to enter/exit the model by penalizing its inclusion/exclusion based on an optimization criterion (6). There are several optimization criteria and among all candidate variables, the next best variable to enter the model is the one which if entered will result in the largest change in the estimated criterion. Regularization-based methods, such the lasso (8) and the elastic net (9) have been extensively applied on 'omics data for feature selection and classification (9, 10). These methods, also referred to as shrinkage methods, select the next best variable to enter the model is the one that would have the most significant coefficient if entered, given all the previous variables selected. The regression coefficients are estimated by penalizing inclusion. Aforementioned methods don't necessarily remove redundant correlation structure between the variables and it has been long argued that they are prone to over-fitting.

(C) Projection-Based Methods.

These techniques are especially suited to deal with a much larger number of correlated variables than samples. They reduce the original number of variables by converting them into new latent variables, which are the non-correlated linear transformations of the original (ie. Principal Component Analysis (PCA) (11) and Partial Least Squares(PLS) regression transforms the data into orthogonal, non-correlated latent components and then uses these components in place of the original variables into a logistic regression model to predict the outcome of interest (12). Nevertheless, these techniques do not perform directly variable selection and in order to do so, further steps needs to be taken, as suggested in the penalized regression PLS (13) and the lasso penalized regression PLS (14). PLS-model based methods applied on gene expression and metabolomics data (i.e. PLS-Discriminant Analysis (PLS-DA) (12) or OPLS-DA (15) an extension of PLS-DA featuring an integrated orthogonal signal correction filter to remove variability not relevant to class separation), eagerly over-fit the data and rigorous validation is necessary to ensure generalization ability (16).

To overcome these challenges, we developed a methodology that combines the statistical efficiency of Partial Least Squares (PLS) in reducing the dimensions of highly correlated datasets with the effectiveness of maximum likelihood estimation in Forward Selection in fitting small models. Our proposed methodology, Forward Selection—Partial Least Squares (FS-PLS) combines the dimensionality reduction of projection-based methods with the model simplicity and clinical interpretability of forward selection stepwise regression. It therefore derives small predictive signatures of the disease or the clinical outcome in question.

### Description of FS-PLS

FS-PLS performs variable selection and model fitting on genome-wide profiles of binary (e.g. 1 if diseased, 0 if healthy control) or linear clinical outcomes (insulin levels). The variables in the model are the measures molecules in an omics dataset, such as the transcript levels in a microarray gene expression experiment, the protein or metabolite intensity peaks in a proteomics or metabolomics study respectively. The algorithm receives as input all the variables included in the study and the goal is to select the minimum set of variables that best classify the clinical outcome of interest.

Given an original set of N molecular variables χ₁ . . . χ_{N} and a clinical outcome y the algorithm FS-PLS initially fits N univariate regression models, y=βᵢxᵢ for i in [1,N]. As in classical regression, the regression coefficient for each model β₁, β₂ . . . β_{N} is estimated using the Maximum Likelihood Estimation (MLE) function, the goodness of fit is assessed by means of a t-test and statistical significance is assessed by comparing the P-value of the t-test statistic with a predefined threshold pₜₕᵣₑₛ (default pₜₕᵣₑₛ=0.05). The first variable to get selected, for example χ₈(N>8), is the one with the highest MLE and smallest P-value. We will call this variable SV₁. Now N−1 variables are left for consideration to enter the final model, which for now contains only SV₁. In order to select the next variable, the algorithm projects out the variation explained by SV₁ using Singular Value Decomposition. The projected variation explained by SV₁, is subtracted from all remaining variables and the algorithm fits N−1 models on the residual variation of each remaining variable. The second variable is selected using the same criteria as for the first one. The algorithm uses this iterative process and at each step the aim is to project out all variations corresponding to the already selected variables and to select a new variable by fitting models on the residual variation. This procedure terminates only when there is no new variable to enter the model with MLE P-value<pₜₕᵣₑₛ. The final model contains all selected variables selected with the regression coefficients as calculated per individual model. No re-fitting of the coefficients is taking place.

There is the option to exclude all variables with variance less than a predefined threshold, in the default setting being var_thresh=0.01.

### Datasets Used in the Study

### Transcriptomics

Leukemia.

The Golub et al. gene expression study collected bone marrow and peripheral blood samples from leukemia patients and the clinical outcome was either Acute Myeloid Leukemia (AML) or Acute Lymphoblastic Leukemia (ALL). The dataset, as described in the original paper (17), has served as a benchmark dataset in several published molecular classification methodologies. The dataset consists of a training set (N=38: 27 ALL 11 AML all bone marrow samples) and an independent test set (N=34: 24 bone marrow and 10 peripheral blood samples) and 7,125 gene expression transcripts were available butafter pre-processing of the data, as described in Dudoit et al. (5), 3,571 transcripts remained as potential biomarkers for disease classification.

Breast Cancer.

FDA approved. The original study is described in The Parker et al. [Parker et al. 2009. J. Clinical Oncology] Breast Cancer “intrinsic” subtyping gene expression study employs the PAM50, a prediction model based on the expression of the 50 classifier genes, to classify subjects into breast cancer intrinsic subtypes. Major intrinsic breast cancer subtypes include Basal-like, Luminal A, Luminal B and HER2-enriched and Normal-like, with each subtype having specific clinical features. The signature of the PAM50 was obtained from an expanded “intrinsic” gene set found in previous microarray studies. The genes were selected so that they have the highest amount of variation between intrinsic subtypes and the least within each subgroup [Peru et al. 2000, Nature]. The employed algorithm, consisted of centroids constructed based on Prediction Analysis of Microarray (PAM) and hence the signature PAM50. The original study is described in Parker et al. 2009. J. Clinical Oncology. In the current study, we used the training set consisting of 225 breast cancers (67 Basal, 77 Luminal A, 34 Luminal B, 35 HER2+ and 12 Normal-like) and the independent test dataset including breast invasive carcinoma expression data (n=547) from The Cancer Genome Atlas TCGA: http://cancergenome.nih.gov/

### Proteomics

### Prostate Cancer.

The Petricoin et al (23) prostate cancer screening trial study from the National Cancer Institute in Maryland aimed to evaluate proteomics as a diagnostics technology to discriminate malignant prostate from benign in men with either normal or elevated PSA levels in the blood. Currently, the amount of Prostate Specific Antigen (PSA) in the blood is followed by a biopsy if recommended, is the common test for prostate cancer detection. Normal PSA levels (serum PSA level <4 ng/mL) suggest healthy prostate while elevated levels (serum PSA level >=4 ng/mL) indicate increased likelihood of cancer but do not distinguish between malignant and benign unless a biopsy confirms it. The proteomics dataset consists of 322 samples, 191 of which with PSA >=4 ng/Ml and confirmed biopsy of malignant prostate, 71 samples with PSA >=4 ng/Ml and confirmed biopsy of benign prostate and 64 samples with PSA<1 ng/MI and healthy prostate. For all samples, SELDI-TOF serum profiling technology generated 15,551 protein peaks.

### Metabolomics

Blood Pressure, Macro- and Micro-Nutrients Metabolomics.

The INTERnational study of MAcro-nutrients, micro-nutrients and blood Pressure (INTERMAP) was a multi-center cross-sectional epidemiologic investigation that was designed to help clarify unanswered questions regarding the role of dietary factors in the development of unfavorable blood pressure (BP) levels in adults. (24, 25) The study included 4,680 participants aged 40 to 59 years from China, Japan, United Kingdom, and United States of America. The data analysed in this paper was collected during two standardized 48-hour dietary recalls (including dietary supplement use), two standardized 7-day histories of alcohol intake, and two timed 24-hour urine samples corresponding to each of the two dietary recalls. Data were discretized into 7,100 spectral bins of equal width (0.001 ppm). For the purpose of the present study, we selected the subset of 1,299 participants of non-Hispanic White ethnicity from eight centers in the United Kingdom and United States of America who were not undertaking treatment for hypertension.

Tuberculosis Diagnostic Transcriptomics Study and RT-PCR Validation.

The case control Tuberculosis in HIV-infected and -uninfected adults from sub-Saharan Africa transcriptomics study aimed at identifying a host whole blood RNA signature to be used to diagnose active tuberculosis (TB) in high HIV/TB prevalence settings from latent TB infection (LTBI) (26). The signature presented in the paper—comprising of 27 transcripts—was derived from microarray expression data acquired from patients recruited in Cape Town and Malawi. The cohort in the original paper as well as this study was split into a training set (N=285), which was used for discovery using elastic net, and a test set (N=76) which was used for validation along with a previously published microarray dataset (N=51) (27). In order to confirm the FS-PLS microarray results across platforms we performed quantitative real-time PCR (qPCR) analysis (even if microarray and qPCR results sometime disagree [(28)].). Measurements for the transcripts of the FS-PLS signature and housekeeping genes (GAPDH and 18S) were acquired using Fluid Dynamic Arrays for the samples of the training and the test set. 272 samples out of the initial 285 of the training microarray set and 74 out of 76 of the test set passed quality control.

### Results

We applied FS-PLS to six published 'omics datasets including two microarray gene-expression transcriptomics, two mass spectrometry proteomics and two Nuclear magnetic resonance spectroscopy (NMR) metabolomics. We also applied FS-PLS on our Tuberculosis transcriptomics diagnostic study and we validated the results, using an independent published cohort and replicated our findings using alternative diagnostic techniques, namely RT-PCR performed extensive comparison the originally employed methods, which include a centroid classifier, the lasso (8), the elastic net (9). We chose to compare our method with methodologies that perform both variable selection and model fitting, as FS-PLS falls within this category. We assume that predictive power is also a function of the data quality and that predictive performance is similar across methods when applied on the same dataset, as demonstrated before (13, 19-21)

### Leukemia

In the original study, the authors used 50 gene transcripts in a self-organizing map (SOM) classifier, which misclassified 2 out of the 38 samples in the training set and 5 out of the 34 samples in the independent test set. However, FS-PLS selected 3 transcripts and achieved perfect discrimination between the two subtypes of leukemia in the leave-one out cross validation of the training set. FSPLS misclassified two samples in the independent test set, which were also misclassified in the original study and it turned out later that those samples were assigned in the wrong class (29). Unsupervised clustering also demonstrates that the 3 genes selected by FSPLS are powerful to naturally group the samples into their real classes. When comparing our results with other published results on the same dataset, Tibshirani and colleagues reported perfect classification between ALL and AML data in the test set by selecting 45 features (30). We also provide the performance metrics of five methods applied to this dataset, which achieve between zero and 4 misclassified samples in the test set. Notably, all methods select more predictors than there samples in the test set and although a rigorous cross-validation procedure was applied, we still argue that selecting more predictors than there are samples to classify is a sign of over-fitting.

### Breast Cancer

In the published PAM50 model, classification was based on the nearest centroid approach. Gene expression data including all intrinsic subtypes were trained over a supervised algorithm to construct centroids for each subtype. These centroids were then used for subtype prediction of the test samples. The distance of the gene expression profile (based on the expression of 50 classifier genes) of each test sample was measured against each subtype centroid. Samples were assigned to the respective intrinsic subtype based on the nearest centroid [Parker et al. 2009. J. Clinical Oncology].

FS-PLS selected a subset of six genes (p-value <0.001) which performed as well as PAM50 gene signature in classifying breast cancer into 5 known intrinsic subtypes. We employed two datasets: 1) the training set which was used to extract the original PAM50 gene signature as obtained from the Gene Expression Omnibus (GEO: GSE10886), and 2) the gene expression profile of the 547 breast invasive carcinoma as downloaded from The Cancer Genome Atlas (http://cancergenome.nih.gov/). We extracted the extended intrinsic gene set from the training data to which FS-PLS was applied.

### Proteomics.

In the original study, the authors used 7 protein peaks to classify the test set, however they did not report the classification algorithm. We therefore compare our method only against the results reported, as well as elastic net. The authors used 56 samples for training and 226 samples for testing; the training set consists of 25 normal prostate and 31 biopsy-proven cancer samples, however the test set consists of 38 biopsy-proven samples and 228 normal or benign prostate samples. The classifier was trained in the two extreme classes and aim at distinguishing the intermediate class, which is benign prostate. Within the benign prostate class, there were 75 samples with PSA<4, which is almost normal, 16 with PSA>10 which would otherwise indicate increased likelihood of malignant prostate and 137 samples in the so called indeterminate class of PSA<4 and 10. FS-PLS selected 5 protein peaks to classify the data. We followed the same design as in the original study).

### Metabolomics

In the original study, linear models were estimated for each spectral bin (as dependent variable) separately, once without adjustment and once adjusting for 11 covariates including study center, gender, and age. A spectral bin was declared to be significantly associated with systolic BP if: (1) the associated p-value was below the Bonferroni-corrected significance threshold controlling the family-wise error rate at the 1% level; (2) the same held true for the two adjacent spectral bins; (3) directions of associations were concordant across the three spectral bins; (4) the previous conditions were satisfied for both visits. In the original paper, the univariate MWAS analysis for systolic BP identified 67 significantly associated spectral bins for unadjusted analyses, and six (three overlapping between the two visits) for adjusted analyses. Analysis of the same dataset using FS-PLS identified a total of 17 significantly associated spectral bins for unadjusted analyses over the two visits (with no overlap, 7 in the first visit and 10 for the second visit), of which five were already identified using the univariate approach described before. As no other multivariate method was applied in the original study, we also performed metabolite selection using the lasso and the elastic net penalized regression to directly compare FSPLS against other powerful similar methods. The three methods, after a split of the data in 80%-20% with a 10-fold Cross-Validation on the training set, achieved almost the same mean-squared error in their predictions, however FSPLS selected only 7 variables compared to 28 and 35 for the lasso and the elastic net respectively.

Elastic net and the lasso tend to allow correlated variables to enter the model even if correlation is not increasing predictive performance, which always results in larger models. FS-PLS through the powerful step of projecting out the explained variation of any new variable, allows correlated variables to enter the model only if they explain additional information in the outcome. We therefore tuned the regularization parameter of elastic net and lasso in such a way as to restrict the maximum number of selected variables to be as many as those selected by FSPLS (i.e. 7 for the first visit and 10 for the second visit dataset). Even in that case, elastic net and lasso selected correlated variables with a slight loss in predictive performance when compared to FSPLS.

We finally adjusted all previous analysis by accounting for eleven covariates, among which the gender, age, smoking, BMI, physical activity and alcohol consumption of the study participants. FSPLS selected 7 variables in both visit datasets, four of which being BMI, gender, age and alcohol in both measurements and in the same order of significance. Of note is the fact that FSPLS selected the metabolite with molecular weight 3.3545 kDa in both datasets. This metabolite was not chosen in the original unadjusted analysis, however the great consistency of FSPLS covariate and spectral bin selection between the two visit datasets serves as a proof of the method's ability to select robust biomarkers.

### Validation and Replication Transcriptomics Study Using Both Microarray Gene-Expression and RT-PCR

In the original study (26), elastic net after 10 fold cross validation for tuning its parameters selected 27 transcripts for the comparison of TB vs LTBI, while FS-PLS selected 3. While the number of transcripts selected is reduced 9 times, the difference between the two classifiers AUC is 1% for the test set and 0.3% for the training set. The transcripts that were selected from elastic net and FS-PLS for the classification of TB vs LTBI in adults were taken forward for RT-PCR validation. Out of the 27 transcripts that elastic net selected, 25 were used for the analysis (one failed quality control and one was represented twice in the signature). The three transcripts that FS-PLS selected passed quality control. The raw CT (cycle threshold) value for every patient and every transcript was acquired and normalized against the mean of the two housekeeping genes (18S and GAPDH) OR quantile normalized to account for biases. The samples for which RT-PCR was run, were divided into training and test set according to the microarray grouping. After using the 25 transcript elastic net model and the 3 transcript FS-PLS model for classification of the RT-PCR data we observed that in general the performance was lower compared to the microarrays. The two methods performed almost identical in terms of classificatory power in both the training and the test set. See Table 1.

### Simulations

Empirical studies on simulated data sets were performed to illustrate the effectiveness of the FS-PLS. The forward selection algorithm (FS), lasso and elastic net were also applied to these data sets as comparisons to FS-PLS. For lasso and elastic net, we used the implementation cv.glmnet in R package glmnet with default parameters.

The root mean square error (RMSE) was employed as an evaluation of the predictions for the data sets with continuous outputs, while the area under ROC curve (AUC) for the data sets with 2-class discrete outputs and the accuracy (ACC) for the data sets with 3- and 5-class discrete outputs. The statistical results for the testing data set show that FS-PLS provided consistently better performance compared to the other three methods. For the few exceptions, FS-PLS still gave competitive results. It is noted that FS-PLS reported dominant performances when the total number of variables or classes is large.

We also studied the number of variables selected by these methods for the final models (regression or classification). FS-PLS selected much less variables for all data sets. Lasso selected about ten times the number of variables as FS-PLS did, and that was even more for elastic net. The differences between FS and FS-PLS were trivial when the total number of variables or classes for the data sets is small, but showed a significant increase then. Interestingly, the number of variables selected by FSPLS remained almost unchanged across data sets TB vs. OD, TB vs. LTBI and INTERMAP, even though the total number of variables had increased from 379 to 7100.

### Discussion

We have developed a novel method for biomarker discovery, FS-PLS, which derives small predictive signatures of disease and clinical outcomes. We have demonstrated the flexibility and applicability of the method using six publically available 'omics datasets, including transcriptomics, proteomics and metabolomics. We showed that FSPLS in all datasets selects a small number of biomarkers with high predictive performance, when directly compared to the original published biomarker selection methodologies. We finally showcased the reproducibility of the biomarkers selected by FSPLS using a Tuberculosis transcriptomics study generated from our lab, whose gene-expression data have already been published (31) and further validated the findings using RT-PCR on the same patients.

On the transcriptomics study of breast cancer, the gene-set obtained by FS-PLS achieved >90% of sensitivity and specificity in terms of classifying the subjects into their respective groups. Molecular biomarkers obtained from gene-expression profiles play an important role in diagnosis and prognosis of cancer patients. However, clinical validation of these signatures has been slow. Shorter signatures and assays with simplified workflow are required for fast and efficient validation of these biomarkers where they can be easily used in clinical practice [Nielsen et al. 2014. BMC Cancer]. PAM50 model is often used for “intrinsic” subtyping of breast cancer. It measures the expression level of 50 classifier genes from breast cancer samples and has been shown to have good prognostic power in both un-treated and tamoxifen treated patients. This model is also used to determine the risk of relapse for each patient [Parker et al. 2009. J. Clinical Oncology]. However, in this study, we only compare molecular subtyping property of PAM50 with FS-PLS generated signature. In the PAM50 signature, there are 10 genes specific to each intrinsic subtype [Parker et al. 2009. J. Clinical Oncology]. With only six genes, FS-PLS performed as well as the PAM50 gene signature. Although these two signatures have been derived from the same source, they have only two genes in common (FOXC1 and ERBB2).

Proteomic profiling on serum or urine samples for biomarker discovery is now coming of age (37). Studies have yielded optimistic results on Alzheimer's disease(38), HIV(39), cancer(40), pancreatitis(41) and Kawasaki disease(42). Applying bioinformatics to proteomics (43) is just emerging. In a recent paper, Zhai et al used support vector machines on SELDI proteomics to derive a 5-protein signature that could discriminate among the different stages of esophageal carcinogenesis. (44) They report 97% specificity with 87% sensitivity. As discussed in a recent review (37), although several biomarkers have been suggested by proteomics studies, few have been actually been validated on a separate cohort or have been discovered in a study that used proper controls. Another major shortcoming has been the lack of appropriate statistical methods for biomarker definition. We expect proteomics technology coupled with biomarker analysis techniques to be in the centre of novel diagnostics. Molecular biomarkers can potentially be used to for diagnosis, disease monitoring or to guide therapy selection. (40) We anticipate such methodologies to be used either as a diagnostic or a molecular decision support tool in distinguishing—at the protein level—diseases, whose accurate diagnosis cannot be achieved using only their clinical features. For example proteomics has successfully yielded results in Kawasaki disease (42), whose etiology is unknown and pathophysiology poorly understood. Kentsis et al showed that by using proteomic profiling they identified 190 potential KD biomarkers almost uniquely present in patients with KD and absent in patients of other clinically-mimicking conditions.

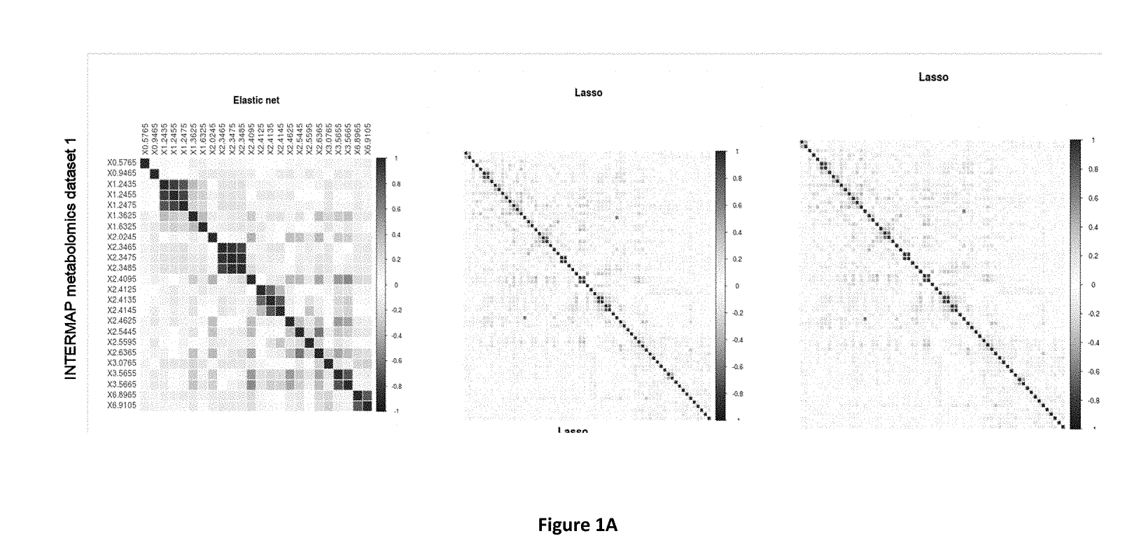

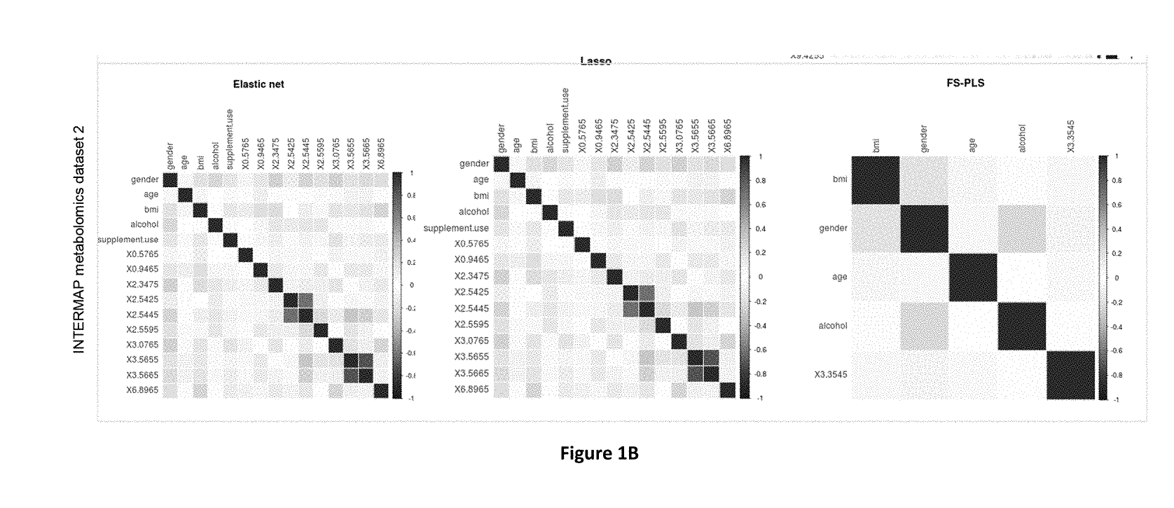

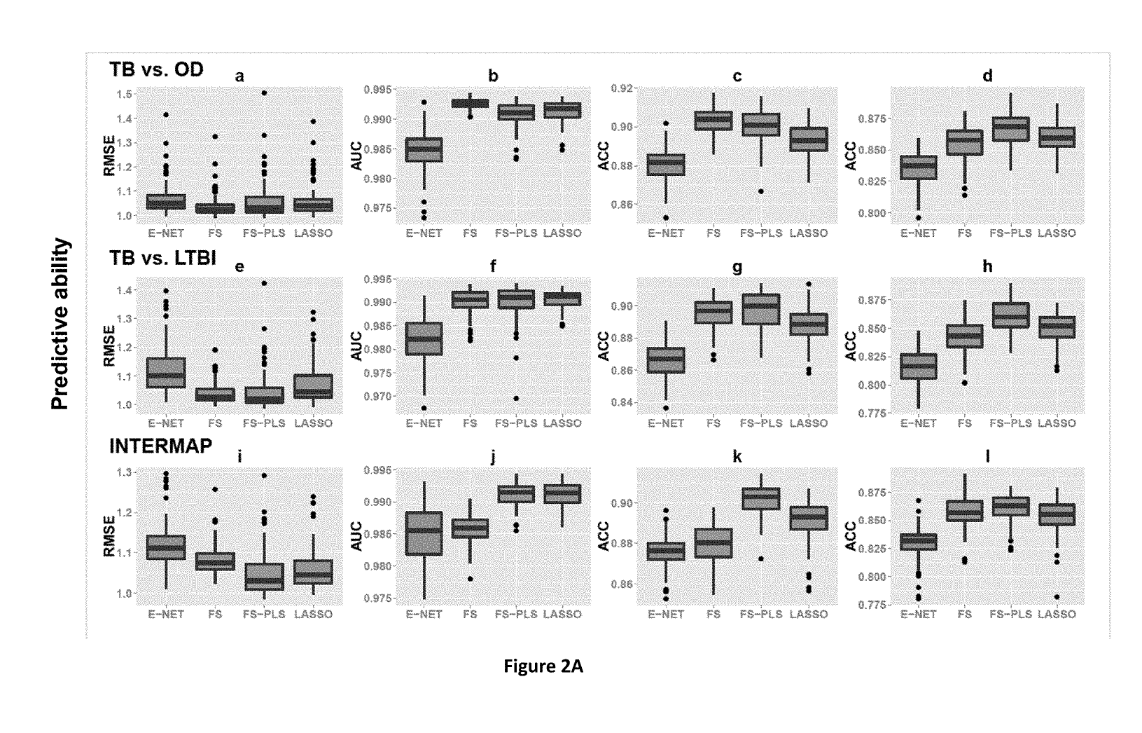

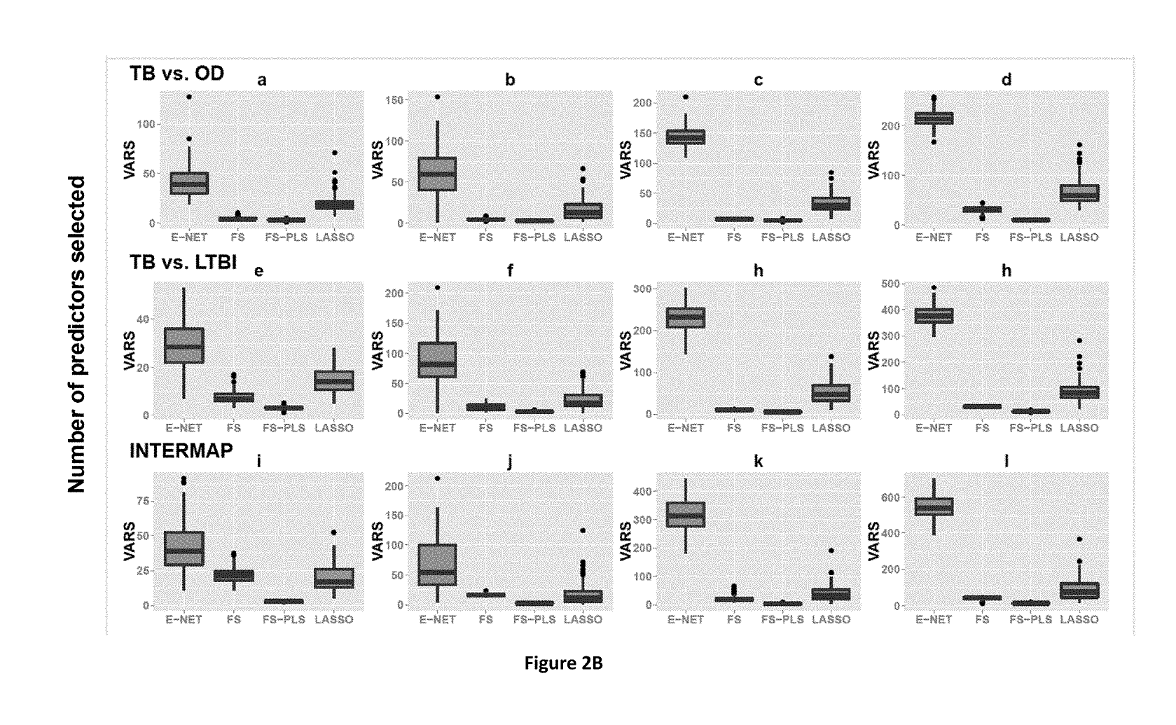

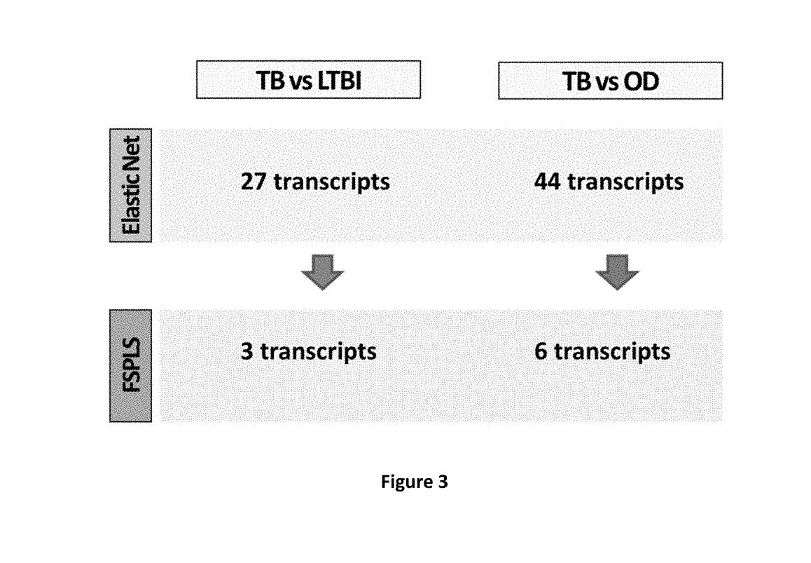

In the metabolomics dataset, strong correlations were observed among significantly associated bins, as exemplified in FIG. 1. This is due to the complex correlation structure that is commonly found in metabolomics data, and consists of three intertwined levels: (1) a local component, reflecting correlations between adjacent spectral bins; (2) a non-local component, reflecting the fact that the same metabolite will usually give rise to multiple (correlated) peaks; (3) a biological component, reflecting the fact that biological processes are usually driven by sets of molecules. Only the latter correlation structure is of interest for biomarker discovery, but it is frequently hidden behind the first two structures. In particular, it is often necessary to apply some unsupervised clustering techniques to identify groups of spectral bins, which can then be characterized chemically. From this point of view, FS-PLS constitutes an important step forward: since the signals it identifies are uncorrelated by construction, they almost certainly originate from different metabolites. These could in turn be easily identified using established techniques in chemometrics such as STOCSY. (36)

We have not applied FS-PLS on genomic as and epigenetics datasets. We expect that these studies will require special adjustment or the evolution of our method to cope with the even higher dimensionality and the low predictive performance. Further work is needed to extend our method for accommodate multinomial response.

FS-PLS has several advantages over various similar methodologies including the fact that (1) it is computationally very fast and applicable to large-scale 'omics data as opposed to traditional FS (2) it is flexible and not platform sensitive, therefore can be readily applied to any 'omics dataset (3) it facilitates clinical interpretation as the outcome is a regression model with weights, as opposed to PLS models that are difficult to understand. It also outperforms similar methods of the PLS family as it (1) directly selects markers rather than the latent components and (2) does not require a further search step within the component loadings (14). FS-PLS therefore achieves interpretability and high predictive power. The small number of predictors also ensures cost-effectiveness in follow-up studies. An important advantage of FS-PLS over all dimensionality reduction methods is the ability to adjust for known confounders, such as age, sex, ethnicity and others. (1) We select un-correlated biomarkers, as it is clearly demonstrated by the correlation plots. (3)

We anticipate our method to find wide application in studies where identifying the minimum set of biomarkers with the highest predictive potential is key for success and cost-effectiveness, such as the field of novel molecular diagnostics. Translating large transcriptomics signatures into clinical diagnostics tools for disease is a complex and expensive process. However there are methods that allow multi-transcript signature measurements (32-35). For all these methods, a reduced number of transcripts would translate into reduced cost and complexity. Molecular classification of diseases using gene-expression profiling has been ongoing for more than a decade with many signatures achieving FDA approval or being transformed into public health diagnostic tools.

### Example 2—Applying FS-PLS Method to Original 44 and 27 Gene Signatures for Detecting Active TB Test Subjects and Validation Datasets

The samples and validation datasets used in this Example are the same as those described in Kaforou et al (26) and in the present inventors' previously filed application WO2014/019977.

### Minimal Gene Signatures

In order to further reduce the number of genes in the original 27 and 44 gene signatures, Forward Selection—Partial Least Squares (FS-PLS) as described in Example 1 was applied to previously obtained gene expression data from Kaforou et al.

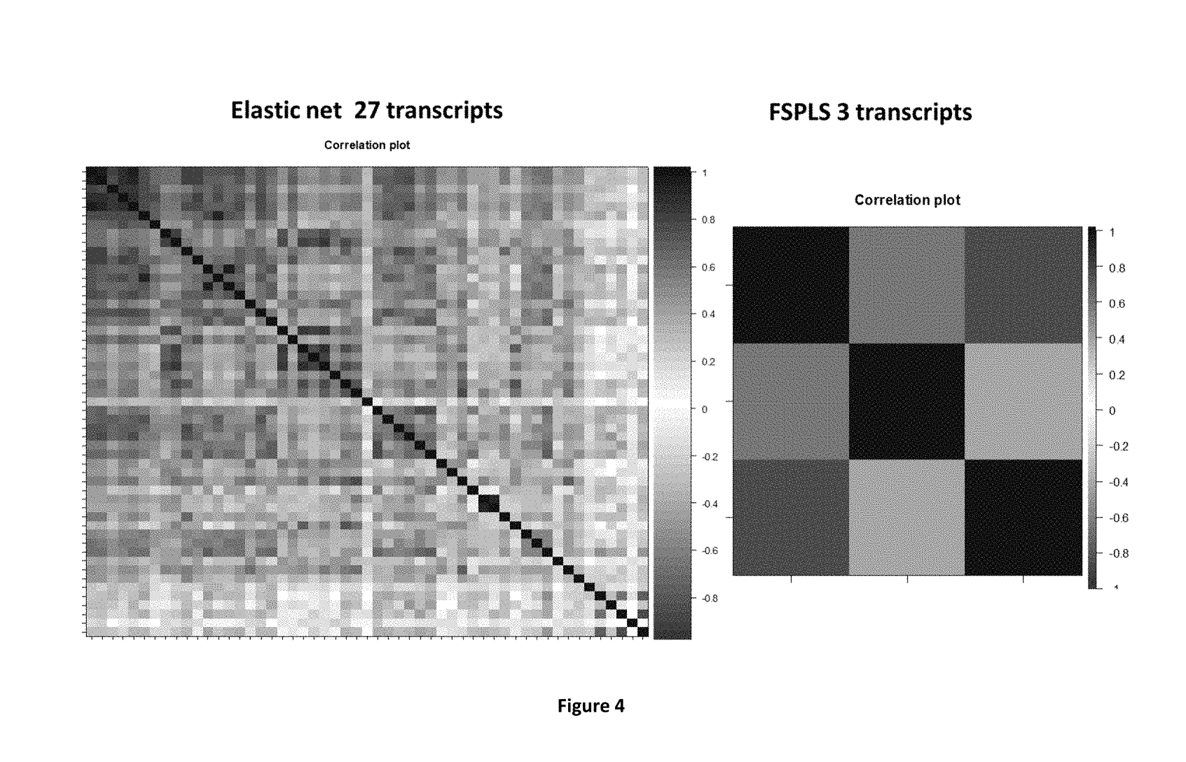

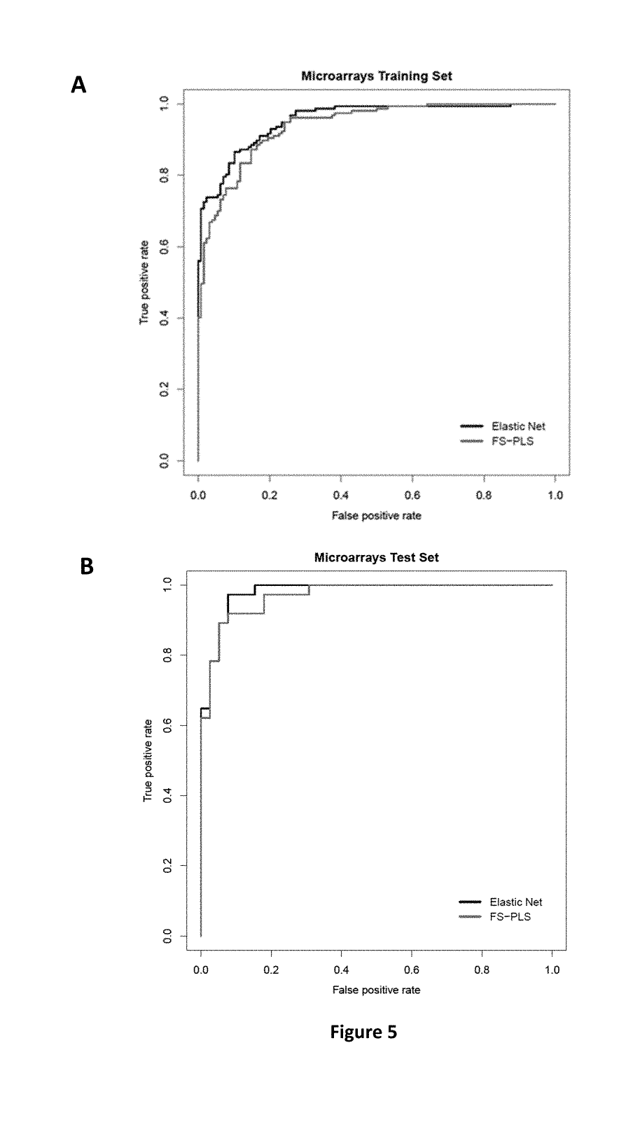

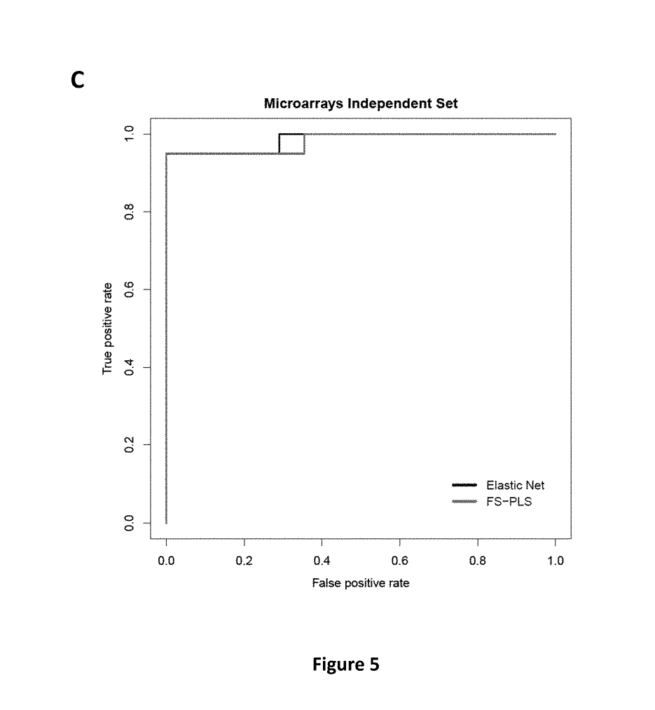

The first iteration of the FS-PLS algorithm considers the expression levels of all transcripts (N) and initially fits N univariate regression models. The regression coefficient for each model is estimated using the Maximum Likelihood Estimation (MLE) function, and the goodness of fit is assessed by means of a t-test. The variable with the highest MLE and smallest p-value is selected first (SV1). Before selecting which of the N−1 remaining variables to use next, the algorithm projects the variation explained by SV1 using Singular Value Decomposition. The algorithm iteratively fits up to N−1 models, at each step projecting the variation corresponding to the already selected variables, and selecting new variables based on the residual variation. This process terminates when the MLE p-value exceeds a pre-defined threshold. The final model includes regression coefficients for all selected variables. See also FIG. 4.

Using FS-PLS, a new minimal 3 gene signature was identified for discriminating between TB and Latent TB, whilst a new minimal 6 gene signature was identified for discriminating between TB and other diseases (see Tables 3 and 4).

### Performance of 3 Gene and 6 Gene Signatures

To evaluate the performance of the new minimal 3 and 6 gene signatures, the disease risk score was calculated. The score is based on subtracting the summed intensities of the down-regulated transcripts from the summed intensities of the up-regulated transcripts. The risk score was calculated on normalised intensities. The disease risk score for individual i is:

$$Disease Risk {Score}^{l} = {\sum }_{k = 0}^{n} expr . {value}_{k}^{l} - {\sum }_{l = 0}^{m} expr . {value}_{l}^{i} \qquad ( 1 )$$

where: n the number of upregulated number of probes in the signature in disease of interest compared to comparator group(s).

m the number of downregulated number of probes in the signature in disease of interest compared to comparator group(s).

The threshold for the classification was calculated as the weighted average of risk score within each class, with weights given as inverse of the standard deviation of the score within each class (1/sd1 and 1/sd2 respectively). The threshold for the classification between group u and v is shown below:

$$threshold ( u , v ) = \frac{\frac{{μ}_{u}}{{σ}_{u}} + \frac{{μ}_{v}}{{σ}_{v}}}{\frac{1}{{σ}_{u}} + \frac{1}{{σ}_{v}}} \qquad ( 2 )$$

where: μ=average of the disease risk score in the group.

σ=standard deviation of the disease risk score in the group.

To calculate the indeterminate zone, we calculated the lower and upper threshold which were calculated as the weighted average with weights given by w/sd1, (1−w)/sd2 respectively for variable 0.5<w<=1. When w=0.5 its equivalent formula to main threshold. ROCs were generated using pROCs. Alternatively:

To calculate the indeterminate zone, we calculated the lower and upper threshold which were calculated as the weighted average with weights given by

$$\frac{w}{{{σ}_{u}}^{′}} , \frac{2 - w}{{σ}_{v}}$$

$$weighted\_threshold ( u , v ) = \frac{w * \frac{{μ}_{u}}{{σ}_{u}} + ( 2 - w ) * \frac{{μ}_{v}}{{σ}_{v}}}{\frac{w}{{σ}_{u}} + \frac{2 - w}{{σ}_{v}}} , 0 \leq w \leq 2 \qquad ( 3 )$$

When w=1 the formula is equivalent to the main threshold formula.

To evaluate the performance of the DRS as a classifier we used different measures (AUC, sensitivity, specificity, PPV, NPV, and likelihood ratios).

The calculation of the confidence intervals for the area under a receiver operating characteristic curve (AUC), the sensitivity and the specificity was based on a non-parametric stratified bootstrap resampling (each replicate contained the same number of cases and controls as the original sample) (Robin et al 2011), with 2000 bootstraps, as recommended by Carpenter et al. (2000).

In order to compare directly the differences of the performance of our signatures to the signatures presented in the Berry et al (2010), we calculated the differences of the means of the measures of classification (namely the AUC, the sensitivity and the specificity) on our test set along with their 95% confidence intervals, using the following mathematical formulas:

$$( a , b ) = {\hat{π}}_{1} - {\hat{π}}_{2} \pm {z}_{a / 2} \cdot s ( D )$$

$$s ( D ) = \sqrt{\frac{{\hat{π}}_{1} ( 1 - {\hat{π}}_{1} )}{{n}_{1}} + \frac{{\hat{π}}_{2} ( 1 - {\hat{π}}_{2} )}{{n}_{2}}}$$

### Results

The results of the performance analyses are shown in Tables 4 to 7. As can be seen from Table 4, the 3 gene signature has a very similar and very high AUC for the training, test and Berry et al validation datasets.

Table 5 shows the results based on RT-PCR validation and likewise indicates that the performance of the 3 gene signature is on par with the performance of the 27 gene signature. Table 6 shows the performance of the individual transcripts of the 6 gene signature. Note that the AUC is very high, which suggests a high discriminatory power for discriminating between active TB and other diseases.

Table 7 shows the results of a comparison of classificatory power for discriminating active TB from other diseases between the original 44 gene signature and the new minimal 6 gene signature.

Again the AUC values are very similar, suggesting that the 6 gene signature has nearly identical discriminatory power as the 44 gene signature.

The results of this Example thus provide a strong indication that the FS-PLS method can effectively identify smaller gene signatures than the previously employed Elastic Net method. This example also demonstrates that testing as few as 3 genes is sufficient to discriminate active TB from latent TB and that as testing as few as 6 genes is sufficient to discriminate active TB from other diseases, even in the presence of a complicating factor such as HIV infection.

### Tables

TABLE 1
Validation and replication study of FS-PLS.
FS-PLS was applied to the Tuberculosis gene-expression study and was
compared to the original method used, which was Elastic Net. Both
Elastic Net (27 gene signature) and FS-PLS (3 gene signature) were
also applied to data derived from the same individuals using RT-PCR.
FS-PLS achieved similar predictive performance while selecting 9 times
less predictors across all comparisons at the replication platform.
Microarrays

| | Training set | | Test set | | Berry et al validation set | |
|---|---|---|---|---|---|---|
| | Elastic | | Elastic | | Elastic | |
| | Net | FS-PLS | Net | FS-PLS | Net | FS-PLS |
| AUC | 0.964 | 0.943 | 0.979 | 0.960 | — | 0.974 |
| CI | 0.9456- | 0.9188- | 0.954- | 0.9150- | — | 0.9285- |
| | 0.9828 | 0.9675 | 1 | 1 | | 1 |

RT-PCR

| | Training set | | Test set | | Combined | |
|---|---|---|---|---|---|---|
| | Elastic | | Elastic | | Elastic | |
| | Net | FS-PLS | Net | FS-PLS | Net | FS-PLS |
| AUC | 0.8553 | 0.852 | 0.9671 | 0.9649 | 0.8657 | 0.8615 |
| CI | 0.8103- | 0.8065- | 0.9276- | 0.924- | 0.8227- | 0.8227- |
| | 0.8975 | 0.8944 | 0.992 | 0.9934 | 0.9038 | 0.8997 |

TABLE 2
27 gene signature and new minimal 3 gene signature

| | | | Direction of |
|---|---|---|---|
| Array ID | Gene Name | Probe ID | regulation* |
| 70730 | GAS6 | ILMN_1779558 | Up |
| 130181 | ANKRD22 | ILMN_1799848 | Up |
| 360132 | LHFPL2 | ILMN_1747744 | Up |
| 520086 | FCGR1A^{#} | ILMN_2176063 | Up |
| 1300139 | GNG7 | ILMN_1728107 | Down |
| 1340241 | C5 | ILMN_1746819 | Up |
| 1440341 | C1QC | ILMN_1785902 | Up |
| 1510026 | FLVCR2 | ILMN_2204876 | Up |
| 1780440 | CD79A | ILMN_1659227 | Down |
| 2630195 | VAMP5 | ILMN_1809467 | Up |
| 2650605 | C4ORF18 | ILMN_1672124 | Up |
| 2710709 | FCGR1B | ILMN_2261600 | Up |
| 2810373 | FAM20A | ILMN_1812091 | Up |
| 2970397 | ZNF296^{#} | ILMN_1693242 | Down |
| 3520601 | MPO | ILMN_1705183 | Up |
| 3780047 | GBP6 | ILMN_1756953 | Up |
| 3890400 | CXCR5 | ILMN_2337928 | Down |
| 4280632 | GAS6 | ILMN_1784749 | Up |
| 5570039 | LOC728744 | ILMN_1654389 | Up |
| 5570398 | FCGR1C | ILMN_3247506 | Up |
| 5890470 | CCR6 | ILMN_1690907 | Down |
| 5910019 | C1QB^{#} | ILMN_1796409 | Up |
| 5910632 | SMARCD3 | ILMN_2309180 | Up |
| 6060468 | S100A8 | ILMN_1729801 | Up |
| 6450594 | CD79B | ILMN_1710017 | Down |
| 6560156 | DUSP3 | ILMN_1797522 | Up |
| 6620209 | FCGR1B | ILMN_2391051 | Up |

*in TB patients in relation to patients with latent TB infection.
^{#}Genes in the 3 gene signature

TABLE 3
44 gene signature and new minimal 6 gene signature

| | | | Direction of |
|---|---|---|---|
| Array ID | Gene Name | Probe ID | regulation* |
| 130086 | CYB561 | ILMN_1771179 | Up |
| 150224 | LOC196752 | ILMN_1803743 | Up |
| 270039 | HM13 | ILMN_1766269 | Up |
| 360132 | LHFPL2 | ILMN_1747744 | Up |
| 380541 | PPPDE2 | ILMN_1737580 | Up |
| 450132 | RBM12B | ILMN_1805778 | Up |
| 450379 | PRDM1^{#} | ILMN_2294784 | Up |
| 540041 | CASC1 | ILMN_1708983 | Up |
| 840446 | CYB561 | ILMN_2378376 | Up |
| 1030433 | CALML4 | ILMN_1815707 | Up |
| 1050360 | HLA-DPB1 | ILMN_1749070 | Up |
| 1070477 | ALDH1A1 | ILMN_2096372 | Up |
| 1110592 | EBF1 | ILMN_1778681 | Down |
| 1170332 | AAK1 | ILMN_1688755 | Up |
| 1580437 | PGA5 | ILMN_1717572 | Down |
| 1690184 | RNF19A | ILMN_1812327 | Up |
| 2000682 | HS.131087 | ILMN_1916292 | Down |
| 2030309 | SERPING1 | ILMN_1670305 | Up |
| 2260349 | MIR1974 | ILMN_3308961 | Up |
| 2340241 | IMPA2 | ILMN_2094061 | Down |
| 2350114 | GJA9 | ILMN_1710161 | Up |
| 2850315 | ORM1 | ILMN_1696584 | Down |
| 3120475 | MAP7 | ILMN_2216815 | Down |
| 3130600 | BTN3A1 | ILMN_1802708 | Up |
| 3310504 | PDK4 | ILMN_1684982 | Down |
| 3360553 | RP5-1022P6.2 | ILMN_1701111 | Down |
| 3780047 | GBP6^{#} | ILMN_1756953 | Up |
| 3840053 | UGP2 | ILMN_1671969 | Up |
| 4070524 | CERKL | ILMN_1801091 | Up |
| 4290619 | CREB5^{#} | ILMN_1728677 | Up |
| 4560047 | CD74 | ILMN_1761464 | Up |
| 4570164 | LOC389386 | ILMN_3215715 | Up |
| 4640768 | VPREB3^{#} | ILMN_1700147 | Down |
| 4670458 | SEPT4 | ILMN_1776157 | Up |
| 5260161 | HS.162734 | ILMN_1893697 | Down |
| 5270753 | ARG1^{#} | ILMN_1812281 | Down |
| 5290100 | MAK | ILMN_1803984 | Down |
| 5820491 | MAP7 | ILMN_1712719 | Down |
| 6380681 | C19ORF12 | ILMN_1664920 | Up |
| 6510754 | ALDH1A1 | ILMN_1709348 | Up |
| 6560156 | DUSP3 | ILMN_1797522 | Up |
| 6760056 | LOC100133800 | ILMN_3287952 | Up |
| 6760471 | TMCC1^{#} | ILMN_1677963 | Down |
| 7210110 | HM13 | ILMN_2236655 | Up |

*in TB patients in relation to patients with other diseases.
^{#}Genes in 6 gene signature

TABLE 4
Comparison of classificatory power for discriminating TB vs
LTBI (latent TB) for 27 gene signature vs 3 gene signature

| | Training Set | | Test Set | | Berry et al validation | |
|---|---|---|---|---|---|---|
| | Elastic Net | FS-PLS | Elastic Net | FS-PLS | Elastic Net | FS-PLS |
| | (27) | (3) | (27) | (3) | (27) | (3) |
| Area Under | 0.96 | 0.94 | 0.98 | 0.97 | 0.99 | 0.98 |
| the Curve | | | | | | |
| 95% CI | 0.93-0.97 | 0.91-0.96 | 0.95-1 | 0.94-1 | 0.95-1 | 0.95-1 |

TABLE 5
Performance of 3 gene signature vs 27 gene
signature based on RT-PCR validation
RT-PCR

Training and Test Sets

| | Elastic Net | FS-PLS |
|---|---|---|
| Area Under the Curve | 0.87 | 0.86 |
| 95% Confidence Interval | 0.82-0.90 | 0.82-0.90 |

TABLE 6
Performance of 6 gene signature

| | Area under the curve in |
|---|---|
| Transcripts | the test set |
| ID1 | 0.85 |
| ID2 | 0.88 |
| ID3 | 0.89 |
| ID4 | 0.92 |
| ID5 | 0.92 |
| ID6 | 0.92 |

TABLE 7
Comparison of classificatory power for discriminating active TB
vs OD (other disease) for 44 gene signature vs 6 gene signature

| | Training Set | | Test Set | |
|---|---|---|---|---|
| | Elastic Net | FS-PLS | Elastic Net | FS-PLS |
| | (44) | (6) | (44) | (6) |
| Area Under | 0.97 | 0.92 | 0.94 | 0.92 |
| the Curve | | | | |

### REFERENCES

WHO report 2011 Global Tuberculosis Control 2011. (http://www.who.int/tb/publications/global_report/en/)
Schultz 2010 Integrative Genomic Profiling of Human Prostate Cancer Cancer Cell Vol 18, Issue 1, 11-22 Metcalfe et al 2010 (“Interferon-γ release assays for active pulmonary tuberculosis diagnosis in adults in low- and middle-income countries: systematic review and meta-analysis” The Journal of infectious diseases 204 Suppl 4).
Berry M P, Graham C M, McNab F W, et al. An interferon-inducible neutrophil-driven blood transcriptional signature in human tuberculosis. Nature 2010; 466:973-7.
Denoeud F, Aury J M, Da Silva C, et al, F; Artiguenave (2008). “Annotating genomes with massive-scale RNA sequencing”. Genome Biol. 9 (12): R175.
Velculescu V E, Zhang L, Vogelstein B, Kinzler K W. (1995) “Serial analysis of gene expression”. Science 270 (5235): 484-7.
Irizarry R A, Hobbs B, Collin F, Beazer-Barclay Y D, Antonellis K J, Scherf U, Speed T P. Exploration, normalization, and summaries of high density oligonucleotide array probe level data. Biostatistics. 2003 April; 4(2):249-64.
Tusher, Virginia Goss; Tibshirani, Robert; Chu, Gilbert (2001). “Significance analysis of microarrays applied to the ionizing radiation response”. Proceedings of the National Academy of Sciences of the United States of America 98 (18): 5116-5121.
Zou, H., and Hastie, T. 2005. Regularization and variable selection via the elastic net. J Roy Stat Soc Ser B 67:301-320. The relevant algorithms of the fully functioning elastic net are incorporates herein by reference.
Crampin A C, Floyd S, Mwaungulu F, et al. Comparison of two versus three smears in identifying culture-positive tuberculosis patients in a rural African setting with high HIV prevalence. Int J Tuberc Lung Dis 2001; 5:994-9.
Hussain R, Kaleem A, Shahid F, et al. Cytokine profiles using whole-blood assays can discriminate between tuberculosis patients and healthy endemic controls in a BCG-vaccinated population. J Immunol Methods 2002; 264:95-108.
Franken K L, Hiemstra H S, van Meijgaarden K E, et al. Purification of his-tagged proteins by immobilized chelate affinity chromatography: the benefits from the use of organic solvent. Protein Expr Purif 2000; 18:95-9.
Benjamini Y, Hochberg Y. Controlling the False Discovery Rate—a Practical and Powerful Approach to Multiple Testing. J Roy Stat Soc B Met 1995; 57:289-300.
Joosten S A, Goeman J J, Sutherland J S, et al. Identification of biomarkers for tuberculosis disease using a novel dual-color R T-MLPA assay. Genes Immun 2012; 13:71-82.
Eldering E, Spek C A, Aberson H L, et al. Expression profiling via novel multiplex assay allows rapid assessment of gene regulation in defined signalling pathways. Nucleic Acids Res 2003; 31:e153.
Maertzdorf J, Ota M, Repsilber D, et al. Functional correlations of pathogenesis-driven gene expression signatures in tuberculosis. PLoS One 2011a; 6:e26938.
Maertzdorf J, Repsilber D, Parida S K, et al. Human gene expression profiles of susceptibility and resistance in tuberculosis. Genes Immun 2011b; 12:15-22.
Jacobsen M, Repsilber D, Gutschmidt A, et al. Candidate biomarkers for discrimination between infection and disease caused by Mycobacterium tuberculosis. J Mol Med (Berl) 2007; 85:613-21.
Cox J A, Lukande R L, Lucas S, Nelson A M, Van Marck E, Colebunders R. Autopsy causes of death in HIV-positive individuals in sub-Saharan Africa and correlation with clinical diagnoses. AIDS Rev 2010; 12:183-94.
Ansari N A, Kombe A H, Kenyon T A, et al. Pathology and causes of death in a group of 128 predominantly HIV-positive patients in Botswana, 1997-1998. Int J Tuberc Lung Dis 2002; 6:55-63.
Maertzdorf J, Weiner J, 3rd, Mollenkopf H J, et al. Common patterns and disease-related signatures in tuberculosis and sarcoidosis. Proc Natl Acad Sci USA 2012; 109:7853-8.
Robin X, Turck N, Hainard A, Tiberti N, Lisacek F, et al. (2011) pROC: an open-source package for R and S+ to analyze and compare ROC curves. BMC Bioinformatics 12: 77.
Carpenter J, Bithell J (2000) Bootstrap confidence intervals: when, which, what? A practical guide for medical statisticians. Stat Med 19: 1141-1164.
Clopper C J, Pearson E S (1934) The use of confidence or fiducial limits illustrated in the case of the binomial. Biometrika 26: 404-413.
Altman D G, Bland J M (1994) Diagnostic tests 2: Predictive values. BMJ 309: 102.
Simel D L, Samsa G P, Matchar D B (1991) Likelihood ratios with confidence: sample size estimation for diagnostic test studies. J Clin Epidemiol 44: 763-770.
1. Ideker T, Galitski T, & Hood L (2001) A new approach to decoding life: Systems biology. Annu. Rev. Genomics Hum. Genet. 2:343-372.
2. Chadeau-Hyam M, et al. (2013) Deciphering the complex: Methodological overview of statistical models to derive OMICS-based biomarkers. Environ. Mol. Mutagen. 54(7):542-557.
3. Guyon I, Weston J, Barnhill S, & Vapnik V (2002) Gene selection for cancer classification using support vector machines. Mach. Learn. 46(1-3):389-422.
4. Geurts P, et al. (2005) Proteomic mass spectra classification using decision tree based ensemble methods. Bioinformatics 21(14):3138-3145.
5. Dudoit S, Fridlyand J, & Speed T P (2002) Comparison of discrimination methods for the classification of tumors using gene expression data. J. Am. Stat. Assoc. 97(457):77-87.
6. Greenland S (1989) MODELING AND VARIABLE SELECTION IN EPIDEMIOLOGIC ANALYSIS. Am. J. Public Health 79(3):340-349.
7. Hoggart C J, Whittaker J C, De lorio M, & Balding D J (2008) Simultaneous Analysis of All SNPs in Genome-Wide and Re-Sequencing Association Studies. PLoS Genet. 4(7):8.
8. Tibshirani R (1996) Regression shrinkage and selection via the Lasso. J. R. Stat. Soc. Ser. B-Methodol. 58(1):267-288.
9. Zou H & Hastie T (2005) Regularization and variable selection via the elastic net. J. R. Stat. Soc. Ser. B-Stat. Methodol. 67:301-320.
10. Zhu J & Hastie T (2004) Classification of gene microarrays by penalized logistic regression. Biostatistics 5(3):427-443.
11. Wold S, Esbensen K, & Geladi P (1987) PRINCIPAL COMPONENT ANALYSIS. Chemometrics Intell. Lab. Syst. 2(1-3):37-52.
12. Barker M & Rayens W (2003) Partial least squares for discrimination. J. Chemometr. 17(3):166-173.
13. Fort G & Lambert-Lacroix S (2005) Classification using partial least squares with penalized logistic regression. Bioinformatics 21(7):1104-1111.
14. Le Cao K A, Rossouw D, Robert-Granie C, & Besse P (2008) A Sparse PLS for Variable Selection when Integrating Omics Data. Stat. Appl. Genet. Mol. Biol. 7(1):32.
15. Bylesjo M, et al. (2006) OPLS discriminant analysis: combining the strengths of PLS-DA and SIMCA classification. J. Chemometr. 20(8-10):341-351.
16. Westerhuis J A, et al. (2008) Assessment of PLSDA cross validation. Metabolomics 4(1):81-89.
17. Golub T R, et al. (1999) Molecular classification of cancer: class discovery and class prediction by gene expression monitoring. Science 286(5439):531-537.
18. Meinshausen N & Buhlmann P (2010) Stability selection. J. R. Stat. Soc. Ser. B-Stat. Methodol. 72:417-473.
19. Huang X H, et al. (2005) A comparative study of discriminating human heart failure etiology using gene expression profiles. BMC Bioinformatics 6:15.
20. Liu H, Li J, & Wong L (2002) A comparative study on feature selection and classification methods using gene expression profiles and proteomic patterns. Genome informatics. International Conference on Genome Informatics 13:51-60.
21. Shi L, et al. (2006) The MicroArray Quality Control (MAQC) project shows inter- and intraplatform reproducibility of gene expression measurements. Nature biotechnology 24(9):1151-1161.
22. van't Veer L I, et al. (2002) Gene expression profiling predicts clinical outcome of breast cancer. Nature 415(6871):530-536.
23. Petricoin E F, et al. (2002) Serum proteomic patterns for detection of prostate cancer. J. Natl. Cancer Inst. 94(20):1576-1578.
24. Stamler J, et al. (2003) INTERMAP: background, aims, design, methods, and descriptive statistics (nondietary). J. Hum. Hypertens. 17(9):591-608.
25. Dennis B, et al. (2003) INTERMAP: the dietary data—process and quality control. J. Hum. Hypertens. 17(9):609-622.
26. Kaforou M, et al. (2013) Detection of tuberculosis in HIV-infected and -uninfected African adults using whole blood RNA expression signatures: a case-control study. PLoS medicine 10(10):e1001538.
27. Berry M P, et al. (2010) An interferon-inducible neutrophil-driven blood transcriptional signature in human tuberculosis. Nature 466(7309):973-977.
28. Morey J S, Ryan J C, & Van Dolah F M (2006) Microarray validation: factors influencing correlation between oligonucleotide microarrays and real-time PCR. Biol Proced Online 8:175-193.
29. Somorjai R L, Dolenko B, & Baumgartner R (2003) Class prediction and discovery using gene microarray and proteomics mass spectroscopy data: curses, caveats, cautions. Bioinformatics 19(12):1484-1491.
30. Tibshirani R, Hastie T, Narasimhan B, & Chu G (2002) Diagnosis of multiple cancer types by shrunken centroids of gene expression. P Natl Acad Sci USA 99(10):6567-6572.
31. Kaforou M, et al. (2013) Detection of Tuberculosis in HIV-Infected and -Uninfected African Adults Using Whole Blood RNA Expression Signatures: A Case-Control Study. PLos Med. 10(10):16.
32. Wang Y, Zheng D L, Tan Q L, Wang M X, & Gu L Q (2011) Nanopore-based detection of circulating microRNAs in lung cancer patients. Nat. Nanotechnol. 6(10):668-674.
33. de la Rica R & Stevens M M (2012) Plasmonic ELISA for the ultrasensitive detection of disease biomarkers with the naked eye. Nat. Nanotechnol. 7(12):821-824.
34. Lowe S B, Dick JAG, Cohen B E, & Stevens M M (2012) Multiplex Sensing of Protease and Kinase Enzyme Activity via Orthogonal Coupling of Quantum Dot Peptide Conjugates. ACS Nano 6(1):851-857.
35. Morrow Ti, Li M W, Kim J, Mayer T S, & Keating C D (2009) Programmed Assembly of DNA-Coated Nanowire Devices. Science 323(5912):352-352.
36. Cloarec O, et al. (2005) Statistical total correlation spectroscopy: An exploratory approach for latent biomarker identification from metabolic H-1 NMR data sets. Anal. Chem. 77(5):1282-1289.
37. Altelaar A F M, Munoz J, & Heck A J R (2013) Next-generation proteomics: towards an integrative view of proteome dynamics. Nat Rev Genet 14(1):35-48.
38. Guo L-H, et al. (2013) Plasma Proteomics for the Identification of Alzheimer Disease. Publish Ahead of Print:10.1097/WAD.1090b1013e31827b31860d31822.
39. Stein D R, Burgener A, & Ball T B (2013) Proteomics as a novel HIV immune monitoring tool. 8(2):140-146 110.1097/COH.1090b1013e32835d33271.
40. de Wit M, Fijneman R J A, Verheul H M W, Meijer G A, & Jimenez C R (2013) Proteomics in colorectal cancer translational research: Biomarker discovery for clinical applications. Clinical Biochemistry (0).
41. Paulo J A, et al. (2011) Mass spectrometry-based proteomics of endoscopically collected pancreatic fluid in chronic pancreatitis research. Proteom. Clin. Appl. 5(3-4):109-120.
42. Kentsis A, et al. (2012) Urine proteomics for discovery of improved diagnostic markers of Kawasaki disease. EMBO Molecular Medicine 5(2):210-220.
43. Mattison H A, Stewart T, & Zhang J (2012) Applying bioinformatics to proteomics: Is machine learning the answer to biomarker discovery for P D and MSA? Movement Disorders 27(13):1595-1597.
44. Zhai X-h, Yu J-k, Lin C, Wang L-d, & Zheng S (2013) Combining proteomics, serum biomarkers and bioinformatics to discriminate between esophageal squamous cell carcinoma and pre-cancerous lesion. Journal of Zhejiang University-Science B 13(12):964-971.

1. A method of treating a subject having active tuberculosis (TB) in the presence of a complicating factor, comprising administering an agent to the subject, wherein the subject has been previously identified as having active TB by detecting in a subject derived sample the modulation in gene expression data, generated from RNA levels in the sample, of the genes in a signature selected from the group consisting of:
a) a 3 gene signature comprising FCGR1A, ZNF296 and C1QB for discriminating active TB from latent TB infection;
b) a 6 gene signature comprising GBP6, TMCC1, PRDM1, ARG1, CREB5 and VPREB3 for discriminating active TB from other diseases; and
c) a combination of signatures a) and b).

2. The method according to claim 1, wherein the complicating factor is the presence of a co-morbidity selected from a malignancy, HIV, malaria, pneumonia, Lower Respiratory Tract Infection, Pneumocystis Jirovecii Pneumonia, pelvic inflammatory disease, Urinary Tract Infection, bacterial or viral meningitis, hepatobiliary disease, cryptococcal meningitis, non-TB pleural effusion, empyema, gastroenteritis, peritonitis, gastric ulcer and gastritis.

3. The method according to claim 2, wherein the co-morbidity is HIV.

4. The method according to claim 1, wherein the genes PRDM1, GBP6 and CREB5 are up-regulated in the subject having active TB compared to a normal subject.

5. The method according to claim 4, wherein the genes VPREB3, ARG1 and TMCC1 are down-regulated in the subject having active TB compared to a normal subject.

6. The method according to claim 1, wherein the genes FCGR1A and C1QB are up-regulated in the subject having active TB compared to a normal subject.

7. The method according to claim 6, example wherein the gene ZNF296 is down-regulated.

8. The method according to claim 1, wherein the subject is previously identified as having active TB by:
a. optionally normalising and/or scaling numeric values of the modulation,
b. taking the normalised and/or scaled numeric values or the raw numeric values, each of which comprise both positive and/or negative numeric values and designating all said numeric values to be negative or alternatively all positive,
c. optionally refining the discriminatory power of one or more up-regulated genes and down-regulated genes by statistically weighting some of the numeric values associated therewith, and
d. summating the positive or negative numeric values obtained from step b) or step c) to provide a composite expression score,
wherein the composite expression score obtained from step d) is compared to a control and the comparison allows the sample to be designated as positive or negative for the active TB.

9. The method according to claim 1, wherein the gene signature further incorporates one or more housekeeping genes.

10. (canceled)

11. The method according to claim 1, wherein the detection of gene expression modulation employs a microarray.

12. The method according to claim 1, wherein the detection of gene expression modulation employs PCR.

13. The method according to claim 12, wherein the PCR is a multiplex PCR.

14. The method according to claim 12, wherein the PCR is quantitative.

15. (canceled)

16. (canceled)

17. A set of primers for use in multiplex PCR wherein the set of primers includes nucleic acid sequences specific to a polynucleotide gene transcript for at least one gene from the group consisting of:
a) FCGR1A, ZNF296 and C1QB; and optionally includes nucleic acid sequences specific to a polynucleotide gene transcript for one or more genes selected from the group consisting of:
b) GBP6, TMCC1, PRDM1, ARG1, CREB5 and VPREB3.

18. The set of primers according to claim 17, wherein the nucleic acid sequences in the set are for no more than a total of 6 genes.

19. The set of primers according to claim 17, further comprising primers specific to one or more housekeeping genes.

20. The set of primers according to claim 17, wherein the primers are specific to a sequence given in any one of SEQ ID NOs: 1 to 16.

21. A point of care test for identifying active TB in a subject comprising the set of primers as defined in claim 17.

22. (canceled)

23. The method according to claim 1, wherein the agent is selected from the group consisting of isoniazid, rifampin, ethambutol, pyrazinamide, streptomycin, kanamycin, amikacin, capreomycin, levofloxacin, moxifloxacin, ofloxacin, para-aminosalicylic acid, cycloserine, terizidone, thionamide, protionamide, clofaximine, linezolid, amoxicillin/clavulanate, thioacetazone, imipenem/cilastatin, high dose isoniazid, clarithromycin.

24. The method according to claim 9, wherein the one or more housekeeping genes are selected from the group consisting of: actin, GAPDH, ubiquitin, 18s rRNA, RPII (POLR2A), TBP, PPIA, GUSB, HSPCB, YWHAZ, SDHA, RPS13, HPRT1 and B4GALT6.
